(19) United States
(12) Patent Application Publication
(10) Pub. No.: US 2023/0287362 A1
(43) Pub. Date: Sep. 14, 2023

(54) METHODS AND COMPOSITIONS FOR THE GENERATION OF PROGRAMABLE POST-TRANSLATIONAL PROTEIN MODIFICATION AND HYDROLYSIS

(71) Applicants: The Regents of the University of Colorado, a body corporate, Denver, CO (US); The Regents of the University of California, Oakland, CA (US)

(72) Inventors: Aaron Whiteley, Boulder, CO (US); Hannah Ledvina, Boulder, CO (US); Kevin Corbett, San Diego, CA (US); Qiaozhen Ye, San Diego, CA (US); Yajie Gu, San Diego, CA (US)

(21) Appl. No.: 18/183,815

(22) Filed: Mar. 14, 2023

Related U.S. Application Data
(60) Provisional application No. 63/319,673, filed on Mar. 14, 2022.

(51) Int. Cl. C12N 9/12; C07K 16/10; C12N 9/10

(52) U.S. Cl. CPC C12N 9/1241; C07K 16/10; C12N 9/1007; C12Y 201/01; C12N 9/1077; C12Y 204/02038; C07K 2319/30

(57) ABSTRACT

The invention describes the discovery and novel application of a bacterial ubiquitin transferase (Cap2). Specifically, the invention describes the novel activity of the enzyme Cap2 which is capable of creating a specific fusion between two proteins implementing a standalone catalytic mechanism to create the fusion.

### CROSS-REFERENCE TO RELATED APPLICATIONS

This U.S. Non-Provisional application claims the benefit of and priority to U.S. Provisional Application No. 63/319,673, filed Mar. 14, 2022. The entire specification, claims, and figures of the above-referenced application is hereby incorporated, in its entirety by reference.

### STATEMENT OF GOVERNMENT INTEREST

This invention was made with government support under grant number R21AI148814 awarded by the National Institutes of Health. The government has certain rights in the invention.

### SEQUENCE LISTING

The instant application contains contents of the electronic sequence listing (90245-00741-Sequence-Listing.xml; Size: 24,500 bytes; and Date of Creation: Mar. 14, 2023) is herein incorporated by reference in its entirety.

### TECHNICAL FIELD

The present invention relates to the fields of molecular and cellular biology, genetic and peptide engineering. In particular the present invention relates to novel systems, methods, and compositions for the generation of programmable post-translational modification and programmable protein hydrolysis

### BACKGROUND

Innate immune pathways rapidly sense and respond to viral threats while limiting their activation in the absence of infection, which could otherwise lead to autoimmune disease or premature cell death. In eukaryotes, viral defense is mediated in part by the cGAS-STING pathway. The cGAS-STING pathway originated from bacterial cyclic oligonucleotide-based antiphage signaling systems (CBASS), which serve an analogous function in the bacterial antiviral immune response. CBASS pathways are diverse and widespread, and protect populations against phage infection by triggering programmed cell death. All CBASS operons encode a CD-NTase that is activated upon phage infection and synthesizes one of a variety of cyclic oligonucleotide second messengers. Those molecules in turn activate a cell-killing effector protein to halt phage replication, a process termed abortive infection.

CBASS operons are classified on the basis of their architecture, with type I CBASS encoding only a CD-NTase and an effector protein, and types II, III and IV encoding additional proteins with proposed regulatory roles. The mechanisms of second messenger synthesis and effector activation in CBASS have been the focus of numerous studies, but the roles of these regulatory proteins in CD-NTase activation remain largely unknown.

Applicants focused on type II CBASS, which make up around 40% of systems, and selected a representative system from a pandemic strain of Vibrio cholerae (FIG. 1a). Upon phage infection, this system's CD-NTase (also known as dinucleotide cyclase in Vibrio (DncV also referred to as cd-Ntase)) (SEQ ID NO. 6) synthesizes the cyclic dinucleotide cGAMP, which activates the cell-killing phospholipase effector CapV (SEQ ID NO. 9). This operon encodes two uncharacterized proteins: CD-NTase-associated proteins 2 and 37 (Cap2 and Cap3) (FIG. 1a). When expressed in Escherichia coli, V. cholerae CBASS confers broad resistance to phage infection (FIG. 1b and Extended Data FIG. 1a,b). Consistent with previous reports, Applicants found that the CD-NTase, CapV and Cap2 are required for resistance, whereas Cap3 is dispensable (FIG. 1b and Extended Data FIG. 1b). To understand CD-NTase regulation in type II CBASS, Applicants immunoprecipitated the CD-NTase from phage-infected bacteria (Extended Data FIG. 1c,d). Mass spectrometry revealed that Cap2 copurified with the CD-NTase, suggesting that these two proteins form a complex (FIG. 1c and Supplementary Table 1) (Notably, all refence to Supplementary Materials, Figures, or Tables includes information found at nature.com/articles/s41586-022-05647-4#Sec27, which is identified and incorporated herein by reference). Applicants confirmed the association between Cap2 and the CD-NTase using reciprocal immunoblots and found that the interaction is independent of phage infection (FIG. 1d,e and Extended Data FIG. 1e-h).

Here, the present inventors show that the CBASS-associated protein Cap2 primes bacterial CD-Ntase for activation through a ubiquitin transferase-like mechanism. A cryoelectron microscopy structure of the Cap2-CD-Ntase complex reveals Cap2 as an all-in-one ubiquitin transferase-like protein, with distinct domains resembling the eukaryotic E1 protein Atg7 and the E2 proteins Atg10/Atg3. The structure captures a reactive-intermediate state with the CD-Ntase C-terminus extending into the Cap2 E1 active site and conjugated to AMP. The present inventors have found that Cap2 catalyzes ligation of the CD-Ntase C-terminus to a target molecule in cells, priming CD-Ntase for a ˜50-fold increase in second messenger production. The present inventors further demonstrated that Cap2 activity is balanced by a specific endopeptidase, Cap3, which deconjugates CD-Ntase and antagonizes antiviral signaling. The present invention demonstrates that bacteria control immune signaling using an ancient, minimized ubiquitin transferase-like system and provide insight into the evolution of E1-E2 enzymes across the kingdoms of life.

### SUMMARY OF THE INVENTION

The invention describes the discovery and novel application of a bacterial ubiquitin transferase (Cap2) and site-specific protease (Cap3). In one preferred aspect, the invention describes the novel activity of the enzyme Cap2 which is capable of creating a specific fusion between two proteins. As shown herein, Cap2 uses a catalytic mechanism similar to known systems of ubiquitin modification but while these systems involve separate E1, E2, and E3 enzymes, Cap2 is capable of performing this reaction on its own. No other standalone protein ligase has been described in the prior art. In another aspect, the invention includes a programmable protein ligation system whereby Cap2 may be used to fuse peptides together.

In one preferred aspect, the invention describes the enzyme Cap3 that is capable of hydrolyzing specific fusion proteins with sequence specificity. In this preferred aspect, the enzyme Cap3 can be used as programmable protease that can be configured to hydrolyze specific peptides in a controlled sequence specific manner.

In another preferred aspect, an engineered Cap2 can tag/modify a protein in vivo. This tag could come in many forms, such as GFP or an epitope tag. In this embodiment, a Cap2 transferase could be engineered such that is modifies a target protein thereby producing a covalently-modified form of this target in a cell, preferably for therapeutic or diagnostic purposes. This technology further allows for detection of proteins without the need for genetic engineering or generation of an antibody specific to the protein of interest.

In another preferred aspect, an engineered Cap2 can be used to generate protein fusions. This embodiment could enable production of proteins that are otherwise too large or otherwise cannot to be produced as one native polypeptide. In this embodiment, the final protein product could be produced in two pieces and Cap2 could joining these fragments together, either in vivo or in vitro.

In another preferred aspect, site-specific protease Cap2 can be engineered to target nucleotide second messenger synthesizing proteins, such as CD-NTase to distinct locations or modify distinct targets in a cell or a whole animal.

In another preferred aspect, site-specific protease Cap3 can be used as a site-specific protease/tool, similar to other proteases, such as TEV protease. In one embodiment, a Cap3 recognition motif could be added to a target protein of interest to mediate Cap3 targeting and cleavage. In this iteration, Cap3 might be used to remove affinity tags post purification, degrade a protein, etc.

In another preferred aspect, site-specific protease Cap3 can be engineered into a biologic to degrade proteins associated with negative outcomes in cells. By generating a Cap3 that specifically degraded a target protein in cells, the invention could be widely used both in basic and translational research.

Additional aspects of the invention are further described the specification, figures, and claims disclosed herein.

### BRIEF DESCRIPTION OF THE FIGURES

FIG. 1 Cap2 is essential for CBASS function and directly interacts with the CD-NTase. a, The operon structure of CBASS from V. cholerae. See Supplementary Table 5 for relevant accession numbers. b, Efficiency of plating of phage T2 when infecting E. coli expressing V. cholerae CBASS with the indicated genotype. Data represent the fold decrease in plaque forming units compared with bacteria expressing an empty vector. See Extended Data FIG. 1b for infections with phages T4, T5 and T6. Catalytically dead (CD) cd-ntase mutation: DID131AIA; C.D. capV mutation: S62A. c, Mass spectrometry of immunoprecipitated VSV-G-CD-NTase. Data are the iBAQ quantification score and fold enrichment comparing immunoprecipitation (IP) with anti-VSV-G from bacteria expressing wild-type CBASS where the CD-NTase has an N-terminal VSV-G tag to a strain expressing the CBASS operon without a VSV-G tag. Cap2 and CD-NTase are represented as colored circles corresponding to a. Circles above the dotted line are proteins with peptides identified only in the VSV-G-tagged samples and not in the untagged control. d, Western blot analysis of immunoprecipitation with anti-VSV-G from E. coli expressing CBASS with the indicated genotypes. ϕ indicates phage T2 at a multiplicity of infection (MOI) of 2. See Extended Data FIG. 1g for analysis of the pre-immunoprecipitation sample. e, Western blot analysis of anti-Flag immunoprecipitation from E. coli expressing CBASS with the indicated genotype. ϕ indicates phage T2 at an MOI of 2. See Extended Data FIG. 1h for analysis of the pre-immunoprecipitation sample.

FIG. 2 Cryo-EM structure of a Cap2-CD-NTase complex. a, Domain schematic of CD-NTase and Cap2 from E. cloacae and ATG10 and ATG7 from Saccharomyces cerevisiae, with domains colored and labelled to represent similarity. E1 and E2 sites are indicated. See also Extended Data FIG. 3. Adn, adenylation residue. b, Cryo-EM density at 2.74 A resolution for the E. cloacae Cap2-CD-NTase (with the CD Cap2 mutation C109A/C548A) complex, with domains colored as in a. a and b represent distinct Cap2 subunits. See also Extended Data FIG. 1i-q, Extended Data Table 1 and Supplementary FIG. 2. c, Two views of the 2:2 heterotetrameric Cap2-CD-NTase complex, with domains colored as in a. d, View of one set of active sites in the Cap2-CD-NTase complex, with the E1 and E2 active site residues (C548 and C109, respectively; both mutated to alanine in this structure) shown as spheres. e, Efficiency of plating of phage T2 when infecting E. coli expressing CBASS with the indicated genotype. Data are plotted as in FIG. 1b. See Extended Data FIG. 4a for infections with phages T4, T5 and T6.

FIG. 3 CD-NTase is the substrate of Cap2. a, The Cap2 adenylation active site in the Cap2-CD-NTase cryo-EM structure. The CD-NTase C terminus (orange) is conjugated to AMP (black). See also Extended Data FIG. 2e-k. b, Sequence logos for the C-terminal 9 residues of 1,556 CD-NTase enzymes from diverse type II CBASS or CD-NTases from the V. cholerae-like group (clade A1; Extended Data FIG. 5d). Data are depicted as bits and signified by the height of each residue. c, Efficiency of plating by phage T2 when infecting E. coli expressing CBASS with the indicated genotype. Data are plotted as in FIG. 1b; see Extended Data FIG. 4b for infections with phages T4, T5 and T6. CD CD-NTase mutation: DID131AIA.d, Western blot analysis of anti-Flag immunoprecipitation from E. coli expressing CBASS with the indicated genotype. e, E. cloacae Cap2 activity assay, representing Cap2-mediated catalysis as a fraction of the activity of the wild type. The indicated genotypes of Cap2 and CD-NTase were expressed from a single plasmid and the formation of a CD-NTase conjugate was measured (in this assay, CD-NTase is conjugated to the flexible N terminus of His6-Cap2; see Extended Data FIG. 4e-k for details). n=3 independent biological replicates; data are mean±s.d. AC, CD-NTase lacking its C-terminal 19 residues; CD, Cap2(C548A/C109A). See Extended Data FIG. 3a for Cap2 protein alignment. f, Western blot analysis of cell lysates from E. coli expressing empty vector (EV) or capV-cd-ntase-cap2 (CBASS Δcap3) with the indicated genotype. g, cGAMP generated by anti-VSV-G immunoprecipitation from E. coli expressing CBASS Δcap3 with the indicated genotype (− indicates CD-NTase without VSV-G; + indicates CD-NTase with N-terminal VSV-G; CD CD-NTase mutation: DID131AIA). See also Extended Data FIG. 5c-f. h, cGAMP generated by anti-VSV-G immunoprecipitation from E. coli expressing capV-(vsv-g-cd-ntase)-cap2. ϕ indicates phage T2 at an MOI of 2. g,h, n=3 technical replicates representative of 3 independent biological replicates. Data are mean±s.e.m.; two-sided student's t-test. NS, P>0.05; *P<0.05 (P=0.0028), **P<0.001 (P<0.0001).

FIG. 4 Cap3 antagonizes CBASS phage defense. a, Efficiency of plating of phage T2 when infecting E. coli expressing CBASS Δcap3 in the absence or presence of overexpressed cap3 with the indicated genotype. Data are plotted as in FIG. 1b. CD cap3 mutation: HTH101ATA. See Extended Data FIG. 6h for protein alignment and Extended Data FIG. 6c for infections with phages T4, T5 and T6. b, Coomassie stained SDS-PAGE of a V. cholerae model substrate (CD-NTase-GFP) incubated with V. cholerae Cap3 with the indicated reaction condition or genotype. See Extended Data FIG. 7 for cleavage of CD-Ntase mutants and for activity assays with E. cloacae Cap3. c, Summary of tryptic digest mass spectrometry analysis of the Cap3-treated CD-NTase bands as in b, showing the putative V. cholerae Cap3 cleavage site. See also Extended Data FIG. 7b and Supplementary Table 3. d, Efficiency of plating of the indicated phage when infecting E. coli expressing CBASS and cap3 from the indicated system. All samples contained 500 μM IPTG to induce Cap3 expression. See also Extended Data FIG. 6d-g. e, Western blot analysis of cell lysates from E. coli expressing CBASS Δcap3 plus a second vector expressing cap3 with the indicated genotype. CD cap3 mutation: HTH101ATA. f, cGAMP generated by anti-VSV-G immunoprecipitation from E. coli expressing CBASS with the indicated genotype (− indicates CD-NTase without VSV-G; + indicates CD-Ntase with N-terminal VSV-G). See also Extended Data FIG. 5c-f n=3 technical replicates representative of 3 independent biological replicates. Data are mean±s.e.m.; two-sided Student's t-test. NS, P>0.05; *P<0.05, **P<0.001 (P<0.0001).

FIG. 5|Proposed mechanism for the role of Cap2 and Cap3 in CBASS signaling. a, Model depicting the role of Cap2 and Cap3 in CBASS regulation. In brief, Cap2 conjugates the CD-NTase to an unknown target via a E1-E2 ubiquitin transferase-like mechanism. CD-NTase conjugation primes the CD-NTase for activation by phage infection. Upon infection, the CD-Ntase becomes enzymatically active and generates a cyclic oligonucleotide second messenger which then activates an effector protein. Effector protein activity leads to cell death, inhibiting phage by abortive infection. This process is antagonized by Cap3 protease activity which removes the CD-NTase from target proteins, thereby limiting priming. b, Top, the general operon structure of type I Pycsar systems. Bottom, sequence logo for the C-terminal 9 residues of 550 PycC proteins encoded within type I Pycsar. See also Supplementary Table 9. c, Top, the general operon structure of identified type II Pycsar systems. Bottom, sequence logo for the C-terminal 9 residues of 55 PycC encoded within type II Pycsar. See also Supplementary Table 9. d, General structures of operons that encode proteins containing E1, E2 and JAB domains. Genes are colored by domain type: blue, E1 and E2 domains; purple, JAB domains; grey, other domains. Genes in dashed boxes are not always found within the operons. Operons are grouped by conserved protein domains and the E1 superfamily of these groups is indicated in parentheses. MBL, metallo-β-lactamase; CEHH, metal-binding domain; multi-ub, tandem p-grasp fold domain-containing protein; ub, single p-grasp fold domain-containing protein. See also Extended Data FIG. 8k and Supplementary Table 9.

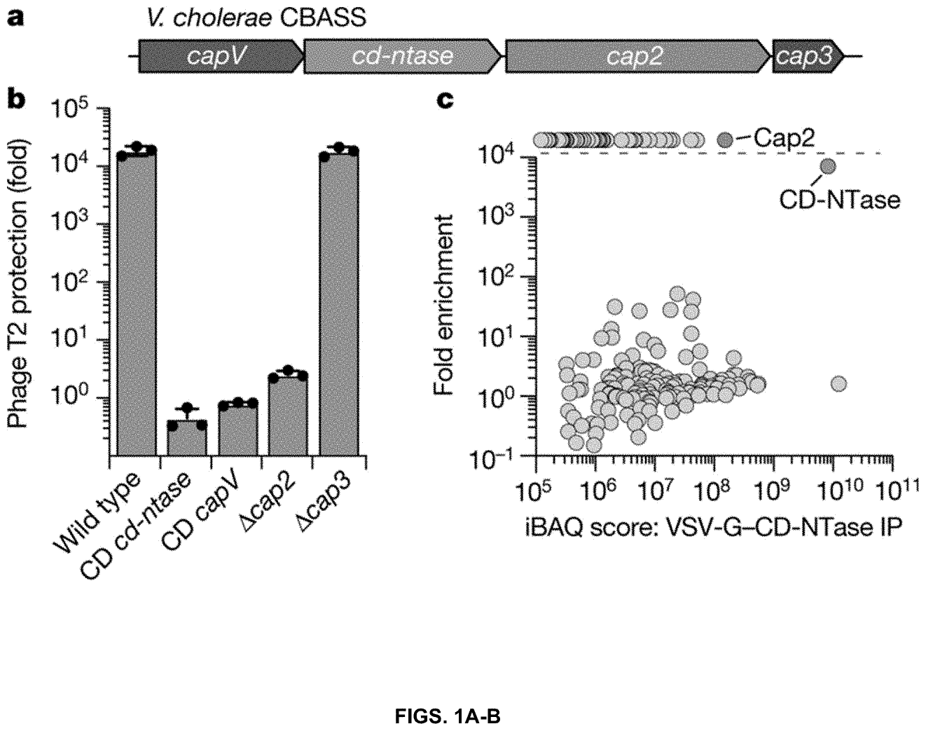

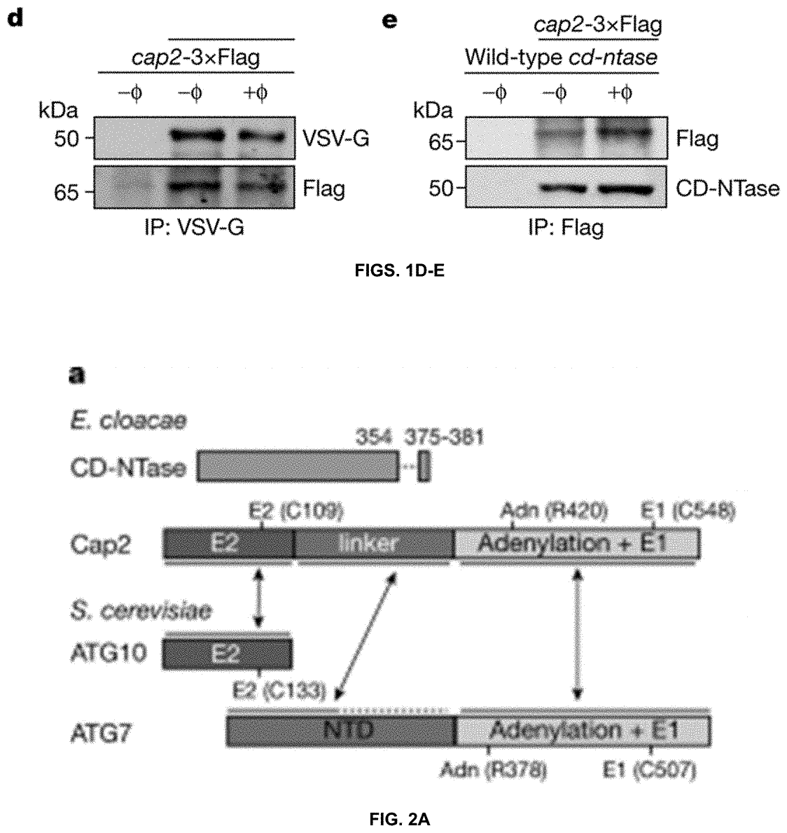

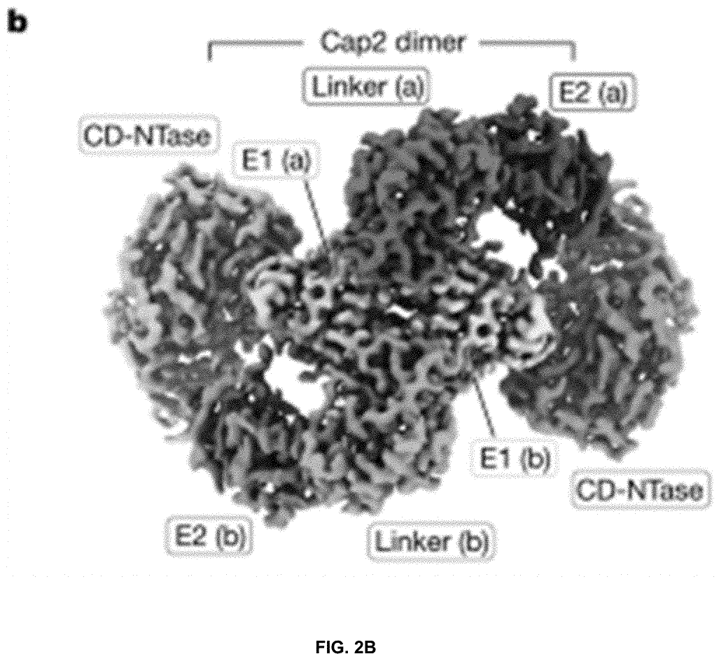

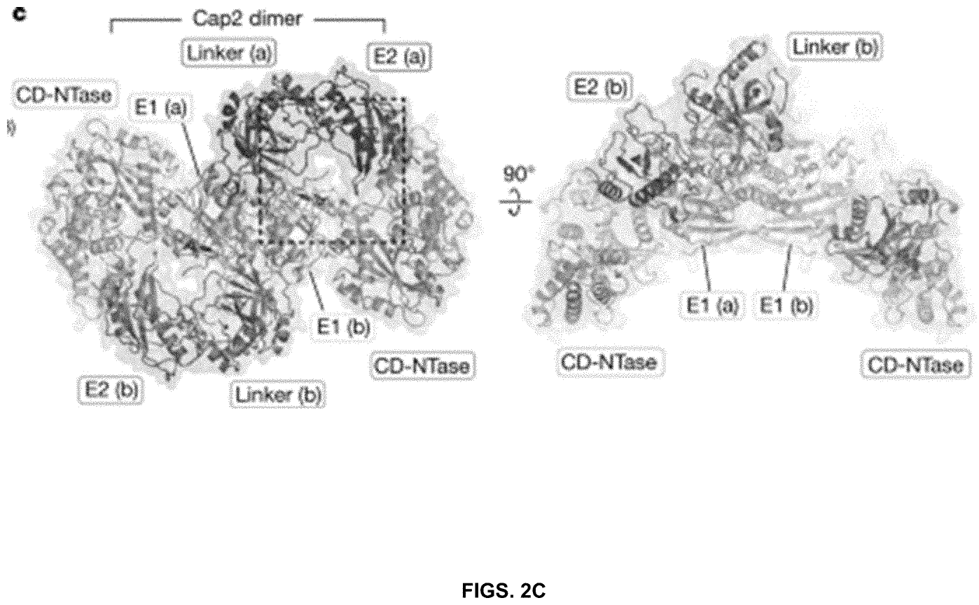

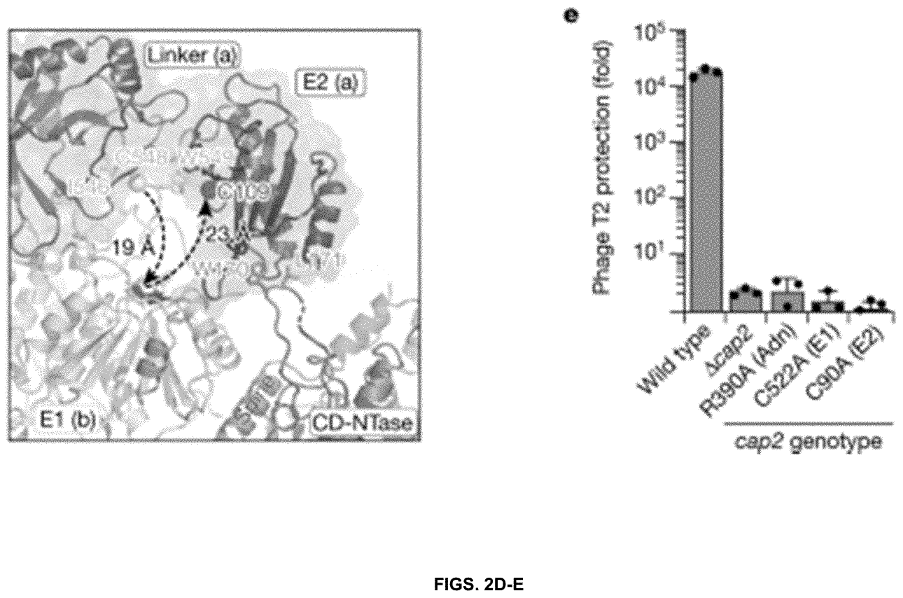

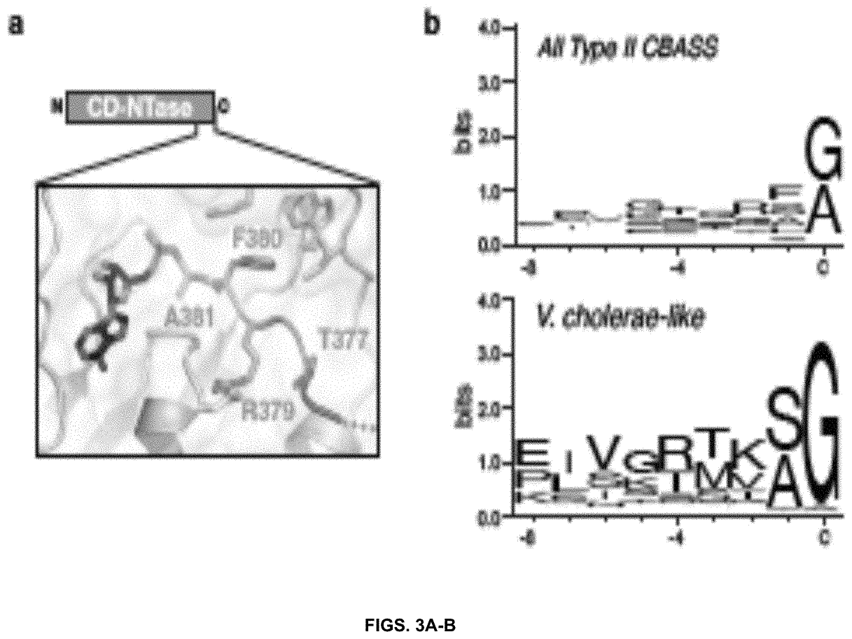

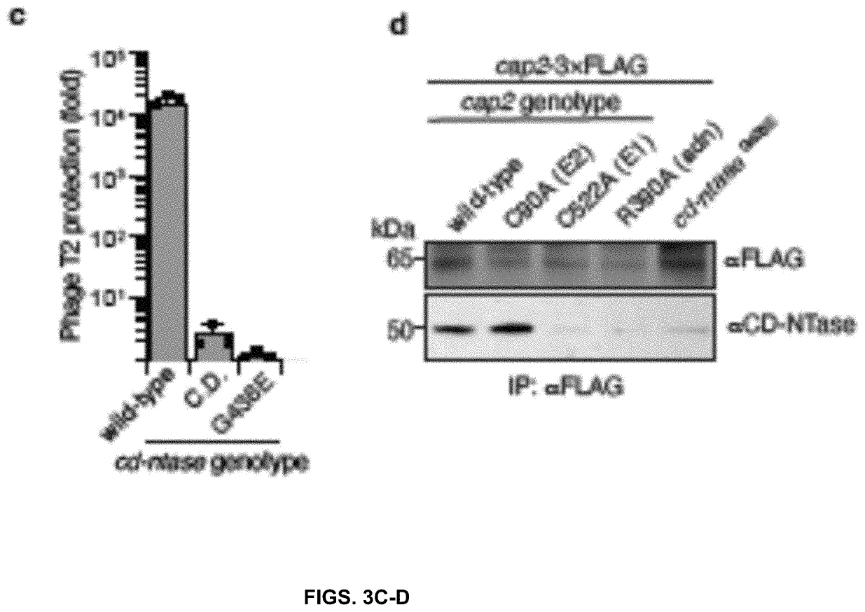

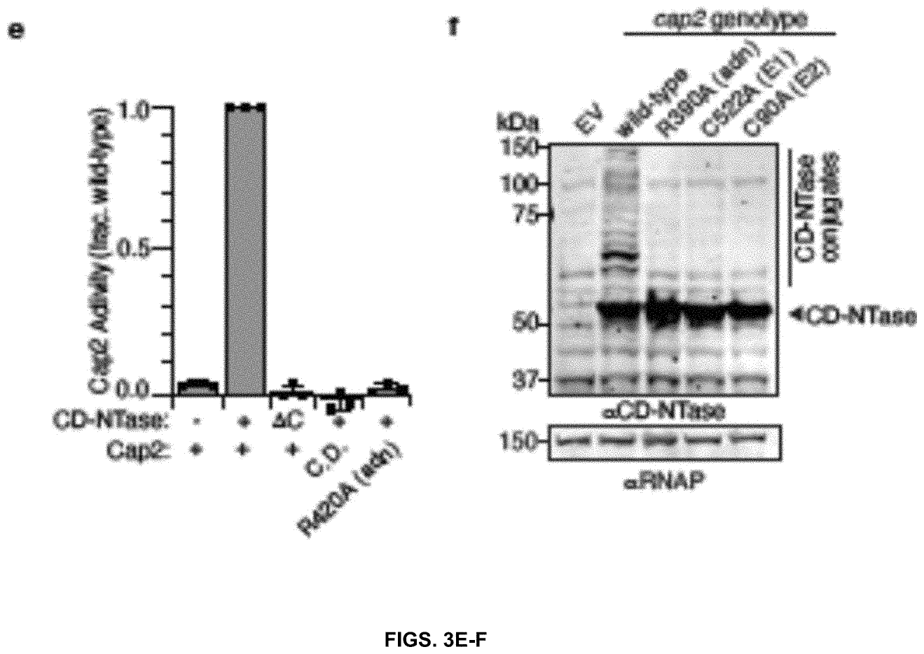

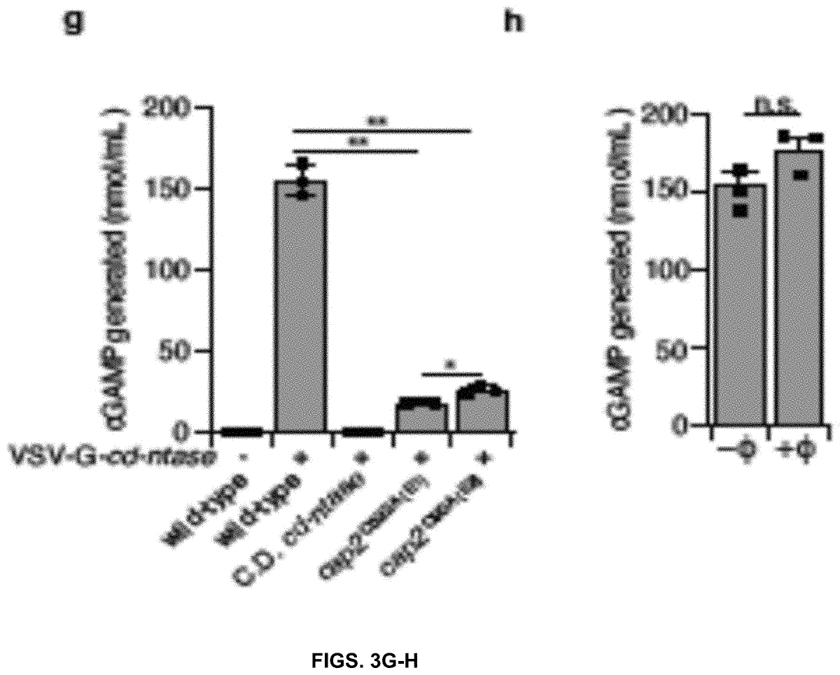

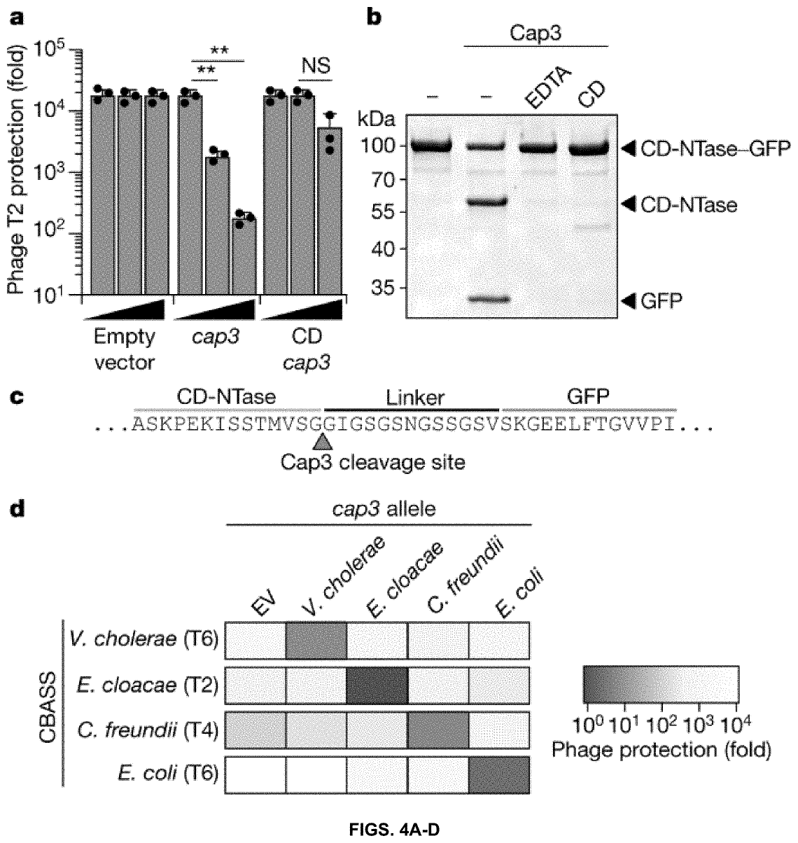

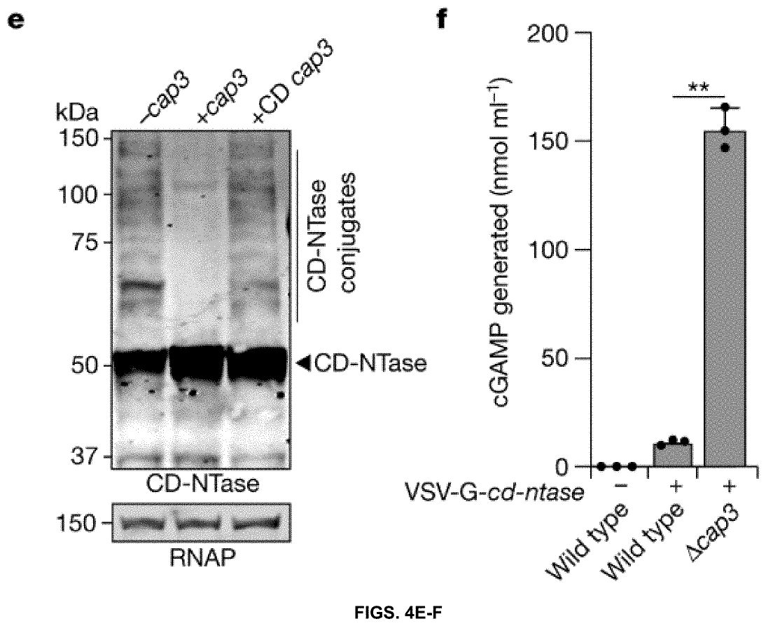

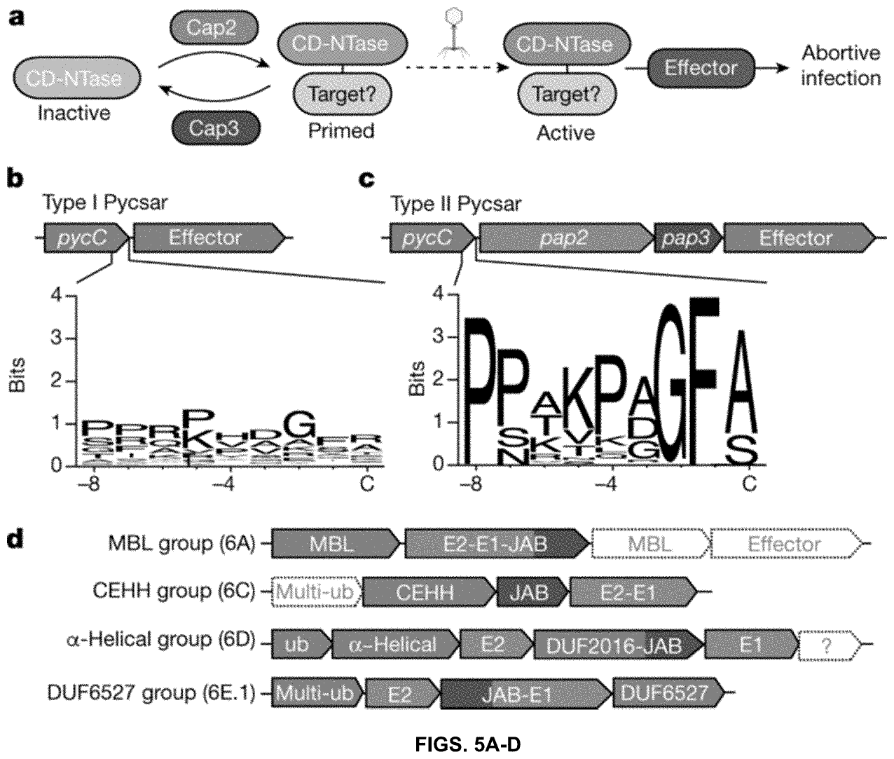

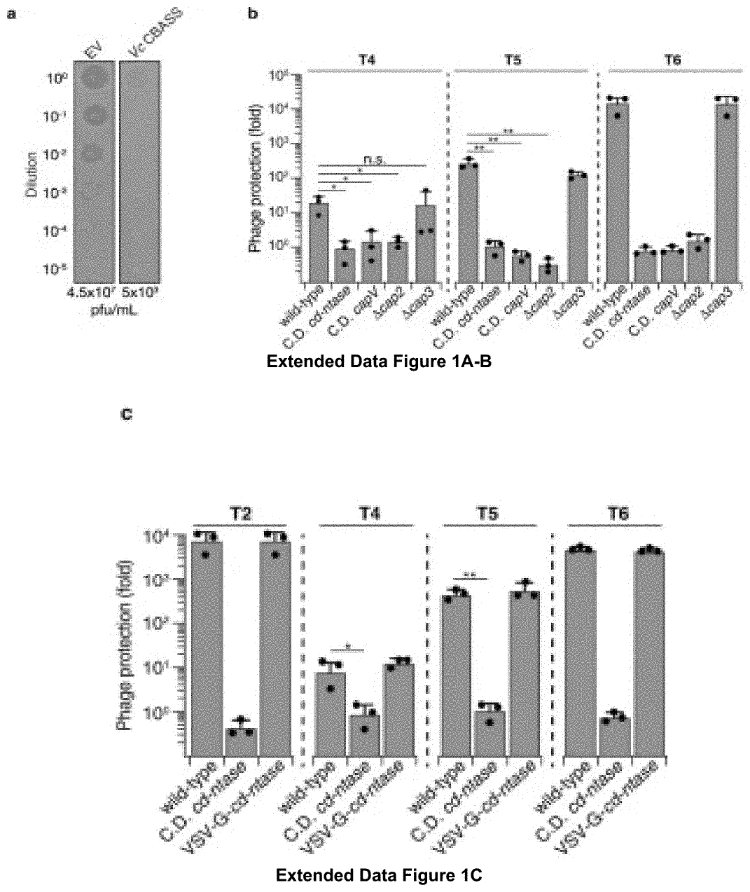

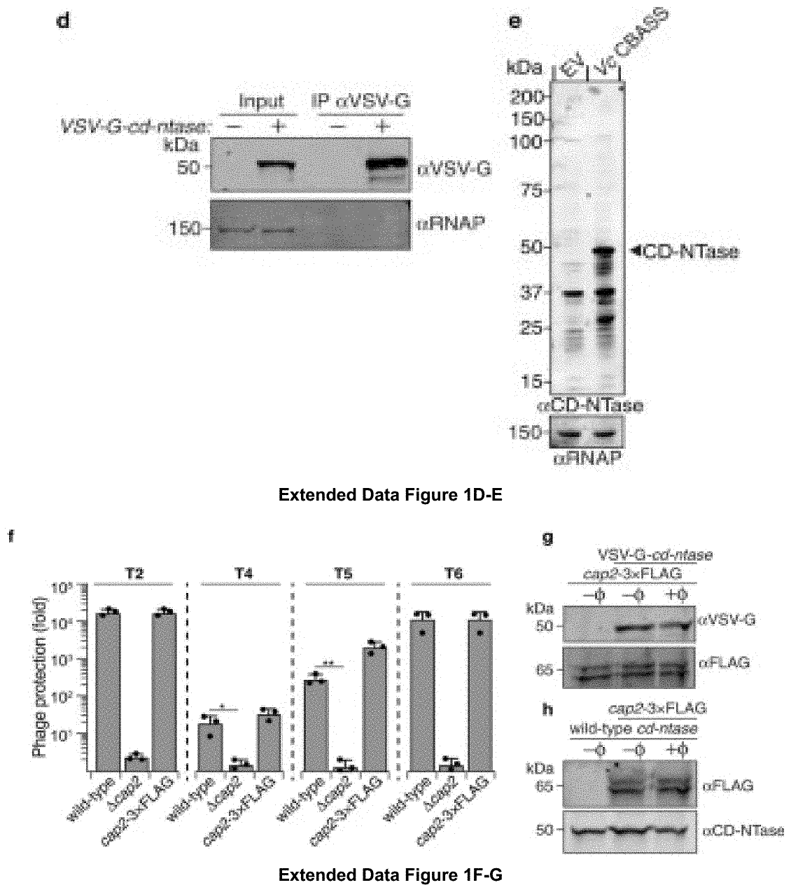

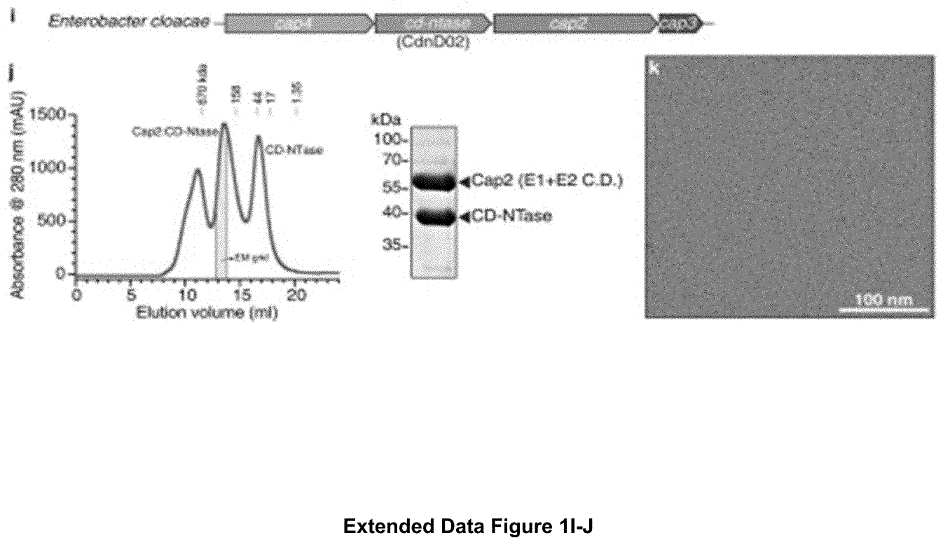

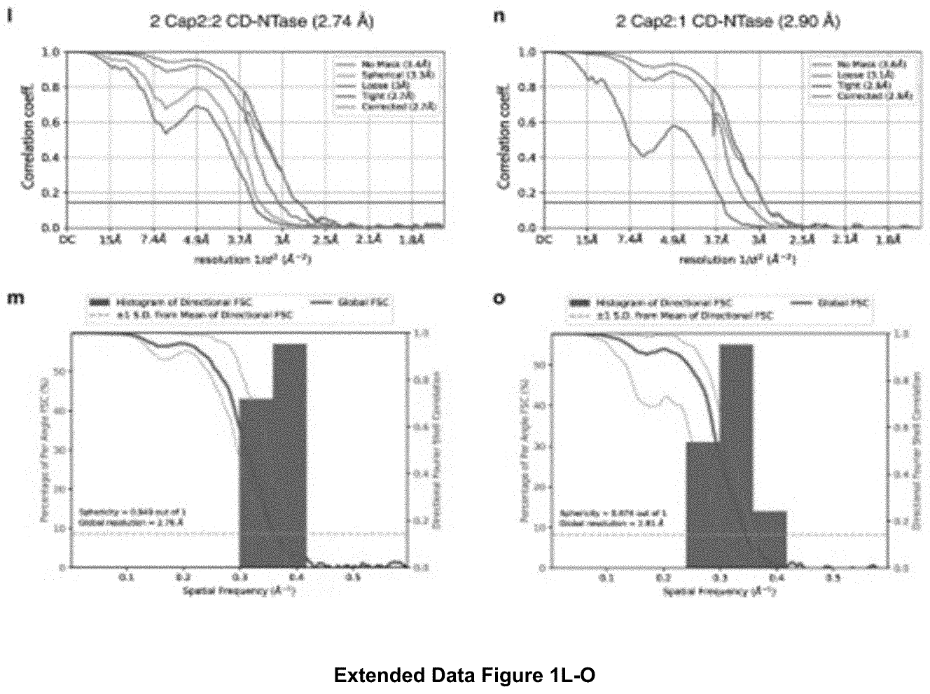

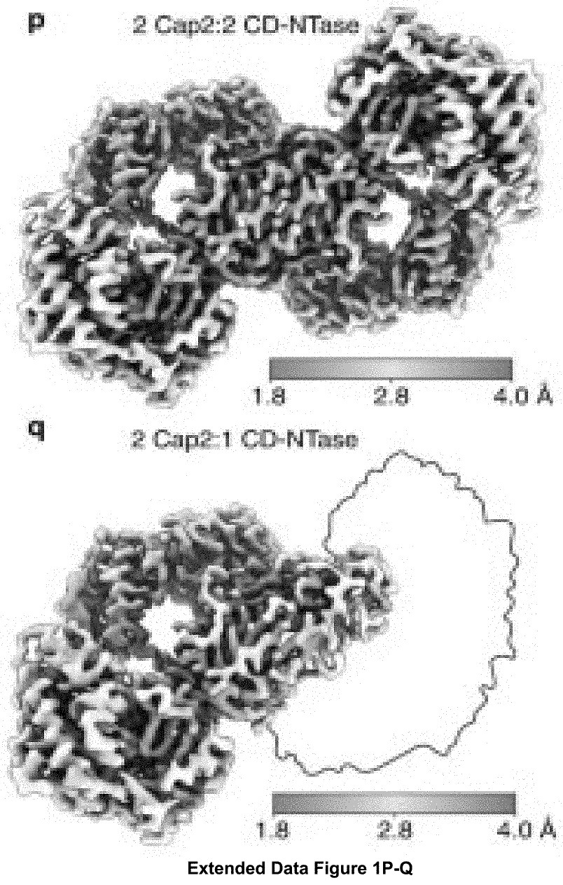

Extended Data FIG. 1 Phage protection assays, inputs for IPs, and CD-NTase antibody verification along with CryoEM information. (a) Image of double agar overlay phage infection assay used to measure efficiency of plating for a lysate of phage T2. E. coli MG1655 expressing the indicated vectors is shown. Zones of clearance (plaques) represent successful phage infection and replication. Apparent plaque forming units (PFU) per mL is calculated for the lysate infecting each bacterial genotype. Fold protection is the PFU per mL of empty vector divided by Vc CBASS, ˜10⁴ in this assay. (b) Efficiency of plating of the indicated phage when infecting E. coli expressing CBASS with the indicated genotype. Data plotted as in FIG. 1b. C.D. CD-NTase: DID131AIA.; C.D. capV: C62A. (c) Efficiency of plating of the indicated phage when infecting E. coli expressing V. cholerae CBASS with the indicated genotypes. Data plotted as in FIG. 1b. (d) Western blot analysis of cell lysates (inputs) and αVSV-G immunoprecipitation of E. coli expressing CBASS with the indicated genotypes. These samples correspond to the mass spectrometry in FIG. 1c. αRNAP western blot serves as a loading control for bacterial cells. (−): CBASS operon, CD-NTase without VSV-G; (+): CBASS operon, CD-NTase with N-terminal VSV-G. (e) Whole cell western blot analysis of E. coli expressing either an empty vector (EV) or CBASS (wild-type). αCD-NTase Western blot used a custom CD-NTase antibody; arrow indicates monomeric CD-NTase at the expected molecular weight. αRNAP western blot serves as a loading control for bacterial cells. (f) Efficiency of plating of the indicated phage when infecting E. coli expressing CBASS with the indicated genotypes. Data plotted as in FIG. 1b. (g) Whole cell western blot analysis of E. coli expressing the indicated genotypes of CBASS. Data are the input for the immunoprecipitation presented in FIG. 1d. (h) Whole cell western blot analysis of E. coli expressing the indicated genotypes of CBASS. Data are the input for the immunoprecipitation presented in FIG. 1e. For (f), (g) and (h)±ϕ indicates phage T2 at an MOI of 2. (i) Operon structure of CBASS from E. cloacae. See Supplementary Table 5 for relevant accession numbers. (j) Size exclusion chromatography elution profile (Superdex 200 Increase 10/300 GL) and SDS-PAGE analysis of E. cloacae Cap2-CD-NTase. The fraction used for cryoEM analysis is shaded in gray. C.D. Cap2: C109A/C548A. (k) Representative electron micrograph of E. cloacae Cap2-CD-NTase. (l) Fourier Shell Correlation (FSC) curve for the final refinement of the 2:2 Cap2-CD-NTase complex. (m) 3D FSC analysis55 for the 2:2 Cap2-CD-NTase complex. (n) Fourier Shell Correlation (FSC) curve for the final refinement of the 2:1 Cap2-CD-NTase complex. (o) 3D FSC analysis for the 2:1 Cap2-CD-NTase complex. (p) Local resolution of the final refined map for the 2:2 Cap2-CD-NTase complex, colored from blue (≤1.8 Å) to magenta (≥4.00 Å). (q) Local resolution of the final refined map for the 2:1 Cap2-CD-NTase complex colored from blue (≤1.8 Å) to magenta (≥4.00 Å). Outline indicates the areas of missing density compared to the 2:2 Cap2-CD-NTase complex.

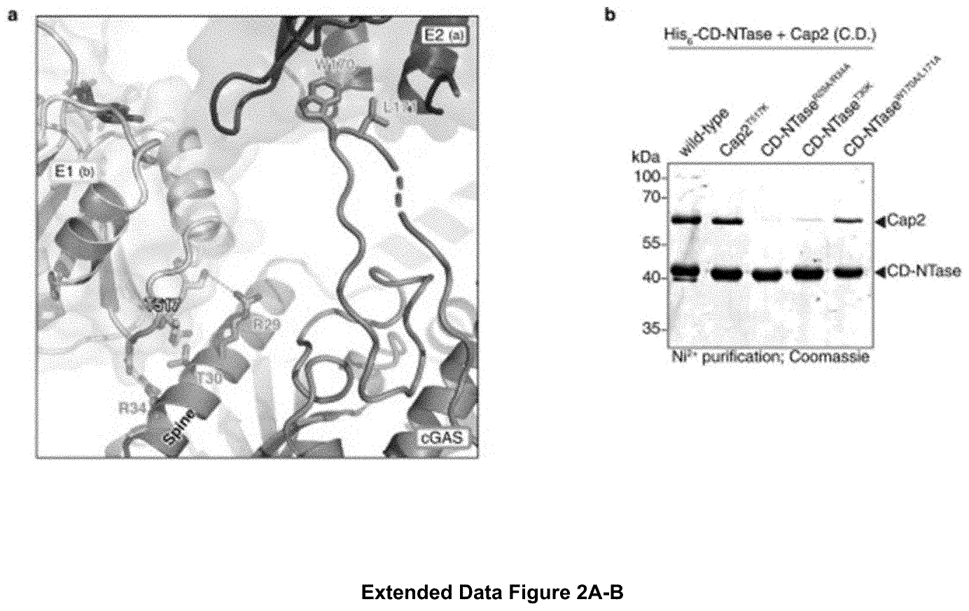

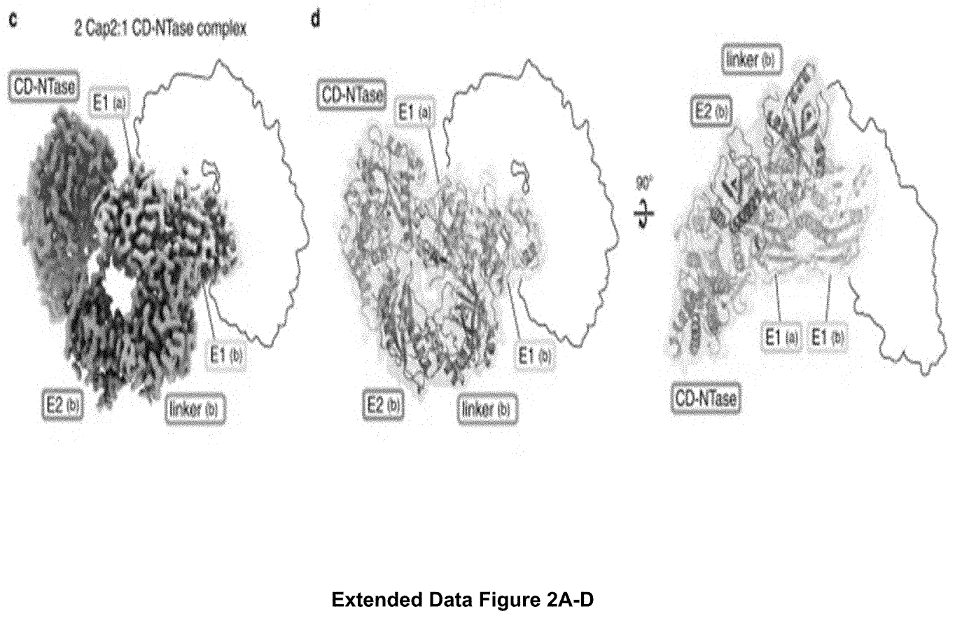

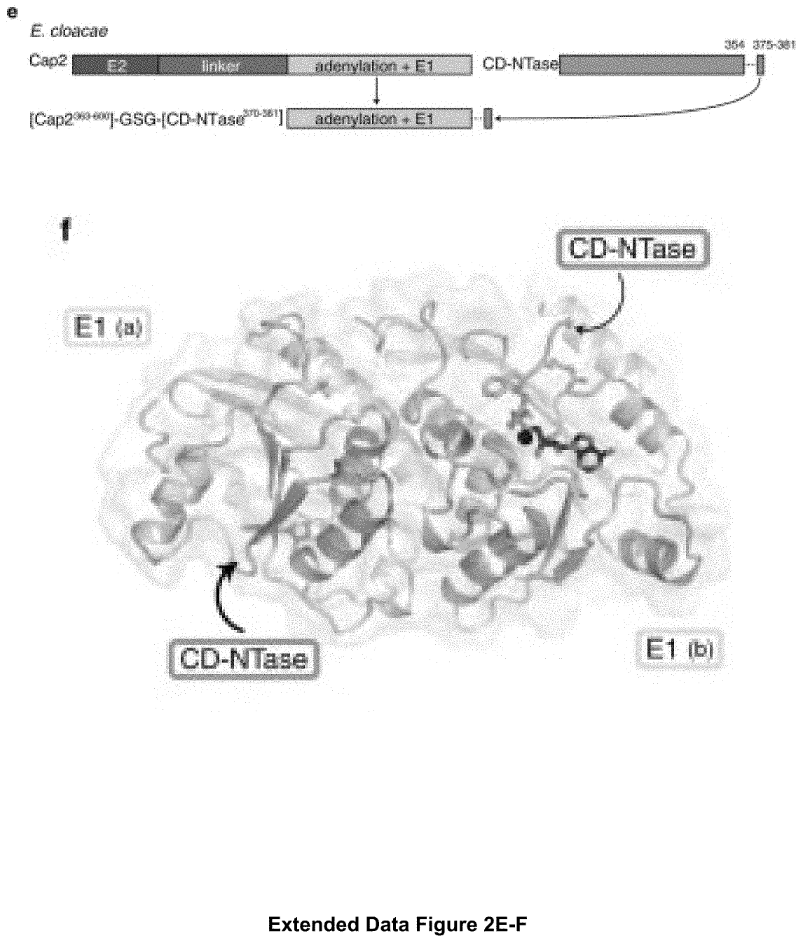

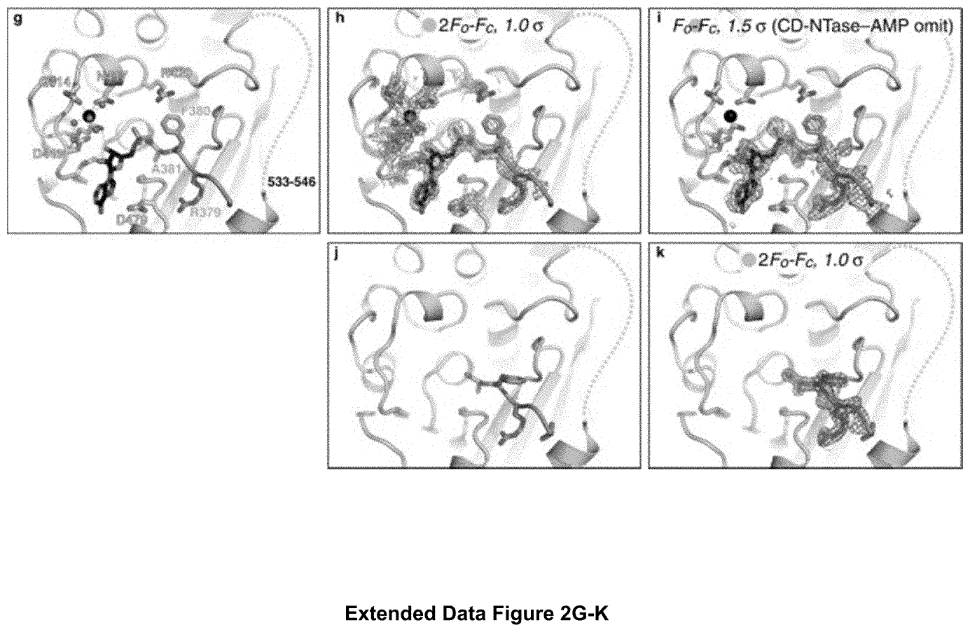

Extended Data FIG. 2 The CD-NTase rigidifies Cap2 through a bipartite interaction and crystal structures of an E. cloacae Cap2 E1-CD-NTase fusion. (a) Closeup view of the interaction between Cap2 (yellow/blue) and CD-NTase (orange), with key residues shown as sticks and labeled. (b) Coomassie stained SDS-PAGE of proteins that purified by Ni²⁺-affinity chromatography from E. coli co-expressing E. cloacae 6×His-tagged cd-ntase (His6-CD-NTase) and catalytically inactivated cap2 (C548A/C109A; C.D.) with the indicated genotype. (c) CryoEM density for 2:1 Cap2-CD-NTase complex, with domains labeled and colored as in FIG. 2b. Outline indicates the areas of missing density compared to the 2:2 Cap2-CD-NTase complex, including one protomer of CD-NTase and the E2 and linker domains for the (a) protomer of Cap2. (d) Two views of the 2:1 Cap2-CD-NTase complex, with domains labeled and colored as in panel (c). Outlines indicate the areas of missing density compared to the 2:2 Cap2-CD-NTase complex. (e) Design of a fusion between the C-terminal E1 domain of E. cloacae Cap2 (residues 363-600) and the C-terminus of CD-NTase (residues 370-381), with a three-residue GSG linker. (f) 2.1 Å resolution crystal structure of the E. cloacae Cap2-CD-NTase fusion crystallized in the presence of ATP, with two Cap2 E1 domains colored yellow and gray, and the two CD-NTase C-termini colored orange. See also Extended Data Table 2. (g) Closeup of the Cap2 adenylation active site, showing the CD-NTase-AMP conjugate and active site residues. Residues 533-546 are disordered and represented by a dotted line. Bound Mg²⁺ ion is shown in black. (h) View as in (g), with 2Fo-Fc electron density contoured at 1.0 σ around the CD-NTase-AMP conjugate and active site residues. (i) View as in (g), with Fo-Fc omit map density contoured at 1.5 σ around the CD-NTase-AMP conjugate. (j) Closeup of the Cap2 adenylation active site in a 1.8 Å-resolution structure of the Cap2-CD-NTase fusion crystallized in the absence of added nucleotide (apo state). (k) View as in (j), with 2Fo-Fc electron density contoured at 1.0 σ around the CD-NTase C-terminus.

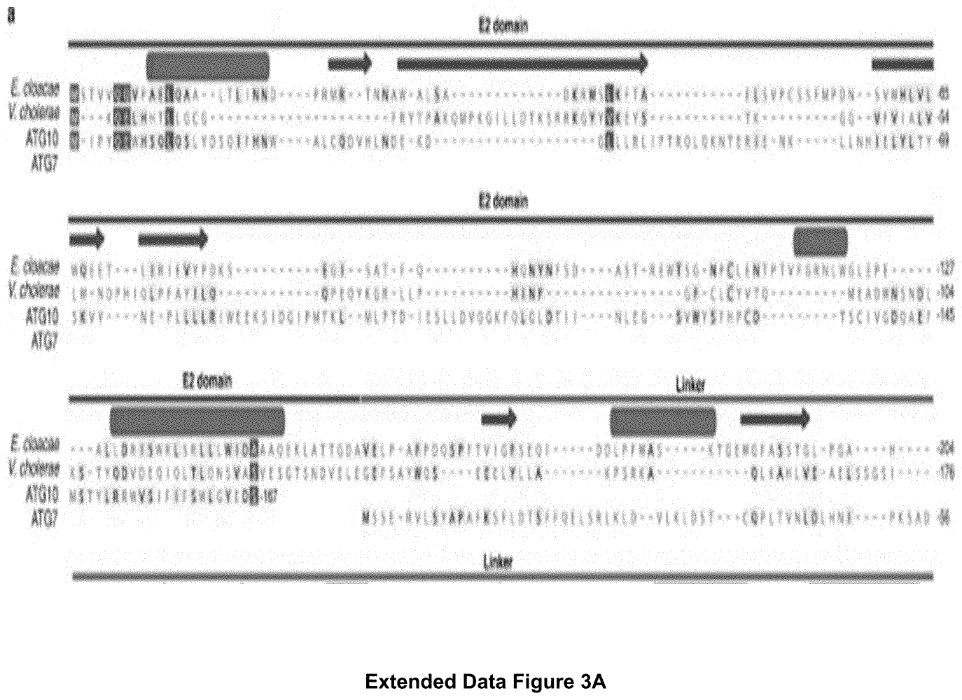

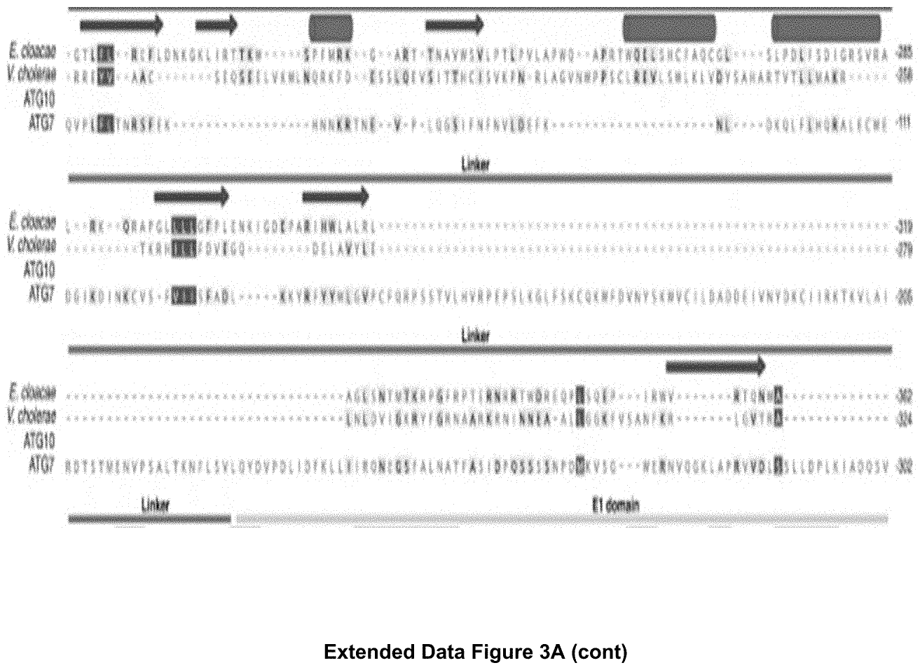

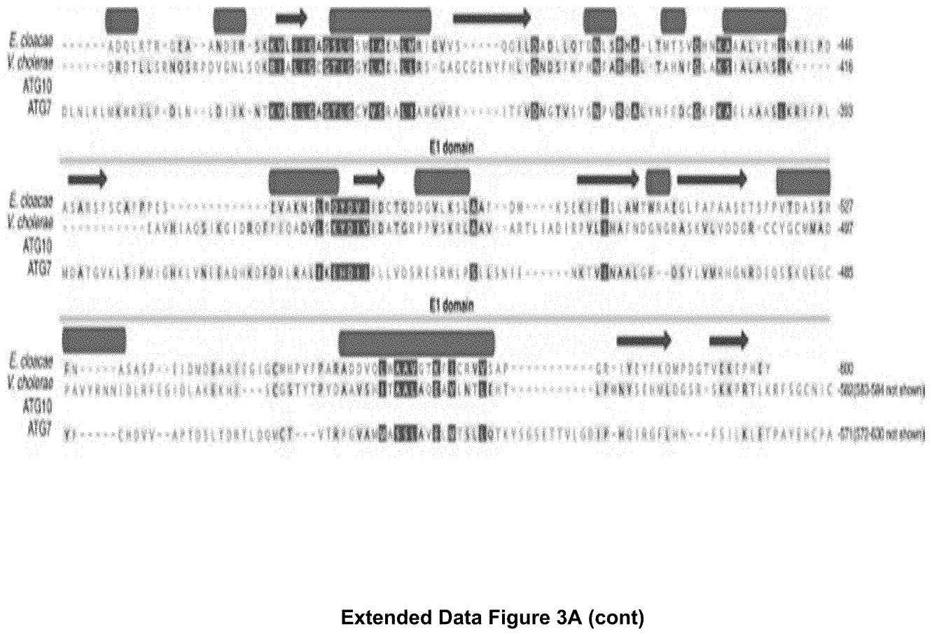

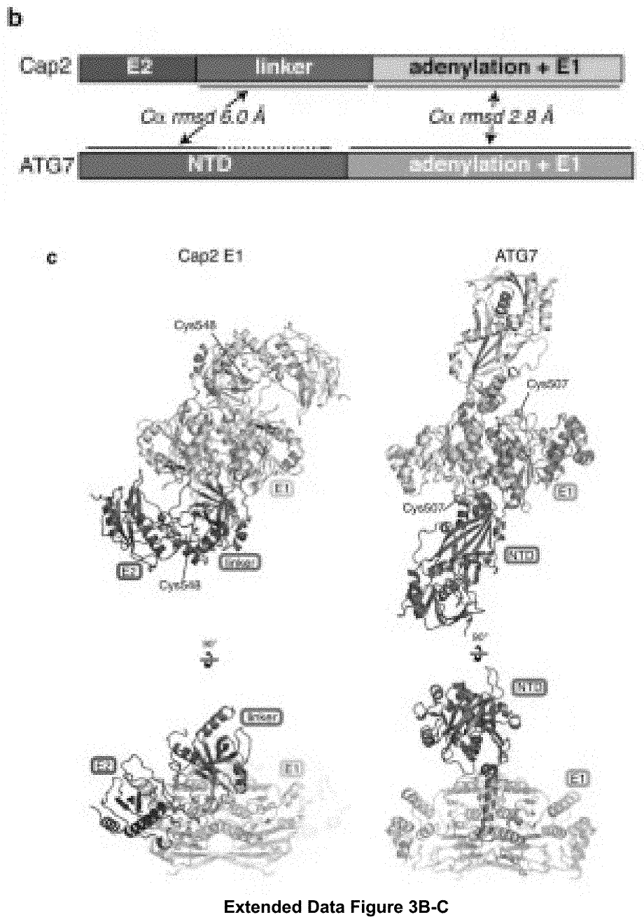

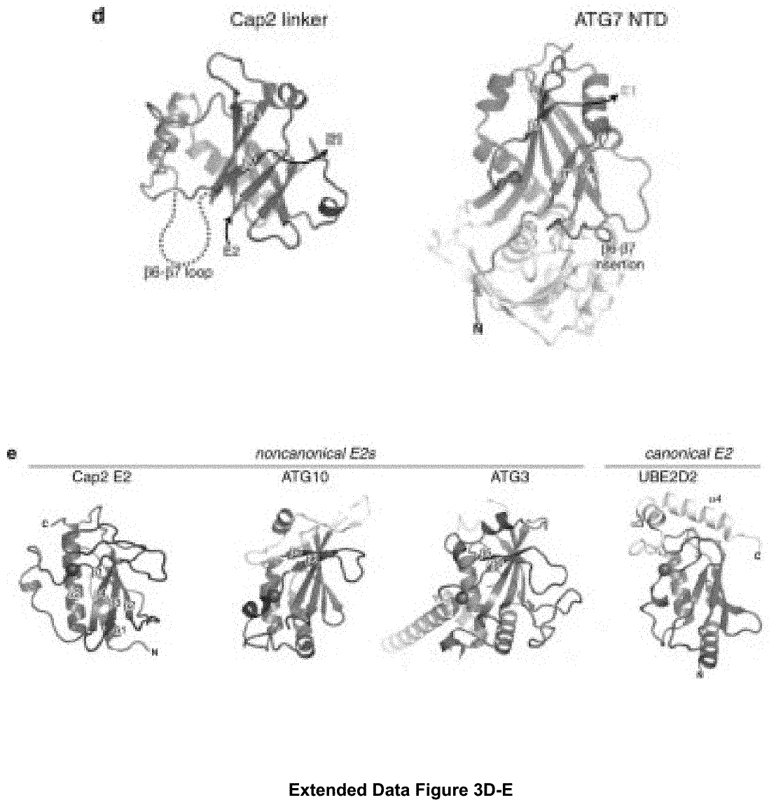

Extended Data FIG. 3 Cap2 is related to autophagy E1 and E2 proteins. (a) Protein alignment of Cap2 from E. cloacae, Cap2 from V. cholerae, ATG10 from S. cerevisiae (4EBR), and ATG7 from S. cerevisiae (3T7H). Domains are indicated above the alignment with colors corresponding to FIG. 2. The secondary structure of Cap2 from E. cloacae is indicated in purple with alpha helices depicted as cylinders and beta sheets as arrows. The catalytic cysteines found in the E2 and E1 domains are highlighted in red. See Supplementary Table 5 for relevant accession numbers. (b) Domain schematic of E. cloacae Cap2 and S. cerevisiae ATG7, with approximate root-mean-squared distance (Cα r.m.s.d.) values for the linker/NTD and E1 domains noted. (c) Structures of E. cloacae Cap2 (left), compared to S. cerevisiae ATG7 (right; PDB ID 4GSK), with one protomer colored as in panel (a) and the dimer mate colored gray. For each protein, the E1 active-site cysteine residue (C548 for Cap2, C507 for ATG7) is shown as a sphere and labeled. (d) Structures of the E. cloacae Cap2 linker domain (left), compared to the S. cerevisiae ATG7 NTD (right; PDB ID 4GSK). ATG7 features a second subdomain (residues 147-268, shown in white) inserted into the loop separating β-strands 6 and 7 (labeled) where Cap2 has a partially disordered loop (residues 319-356). (e) Structure of the Cap2 E2 domain (active-site C109 shown as a sphere), compared to Kluyveromyces marxianus ATG10 (PDB ID 3VX7), S. cerevisiae ATG3 (PDB ID 2DYT), and Homo sapiens UBE2D2 (PDB ID 4DDG). Structural features not shared are shown in white. The active-site cysteine of each protein is shown as a sphere.

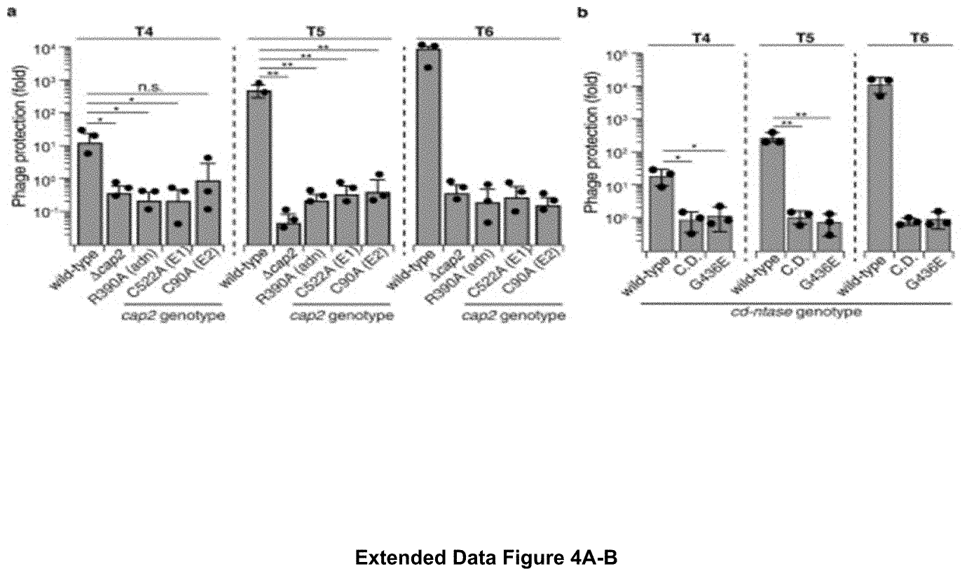

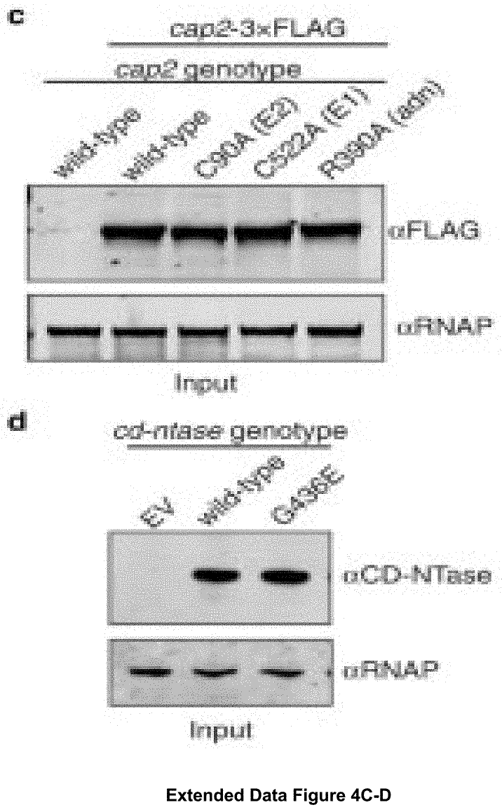

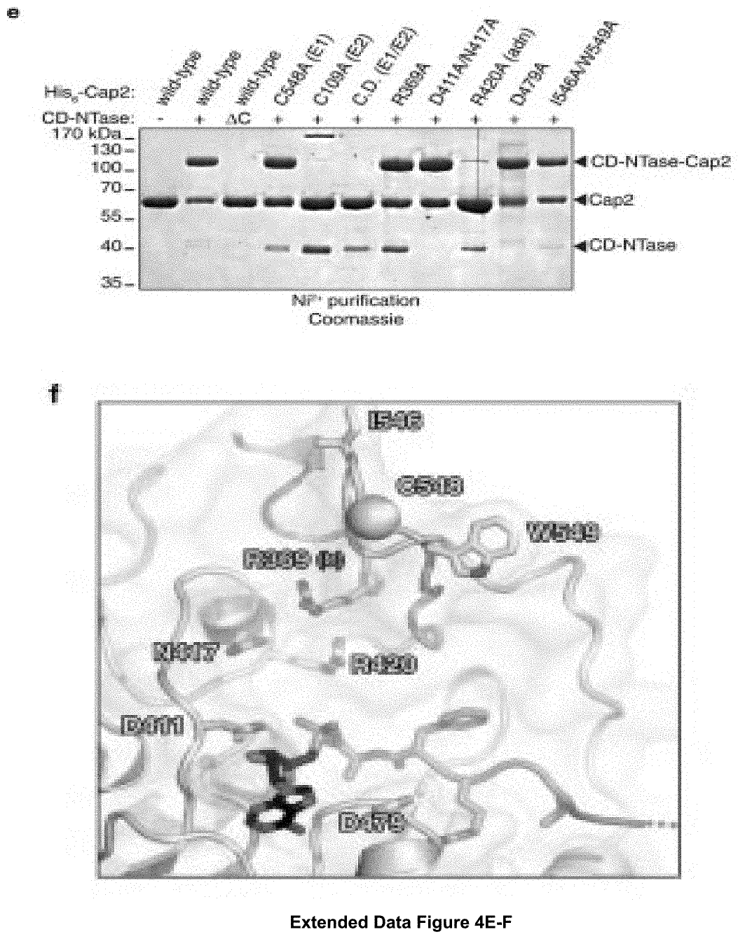

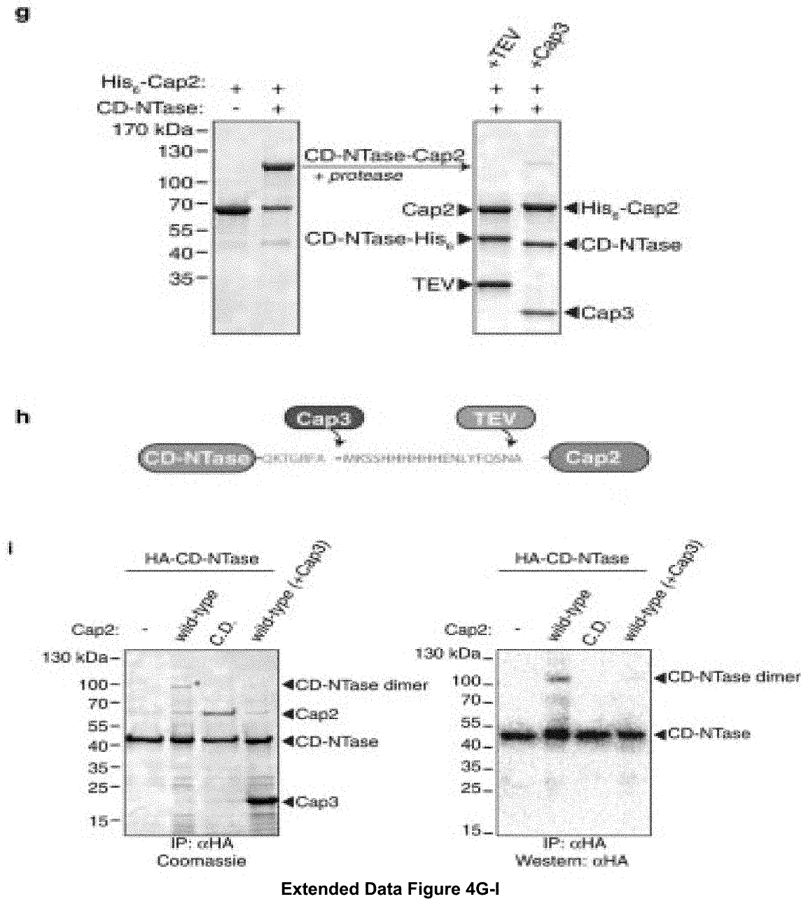

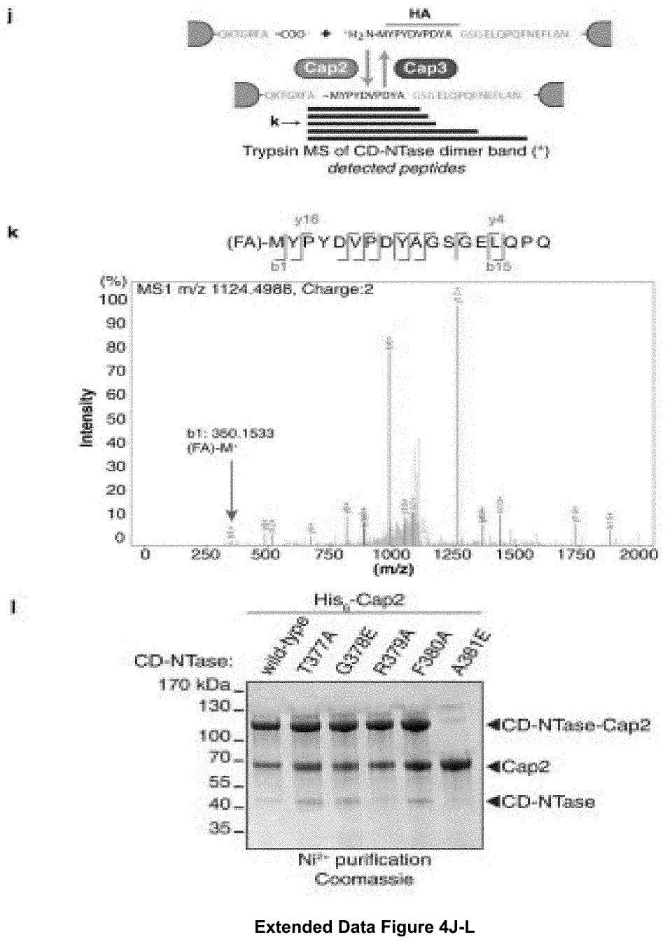

Extended Data FIG. 4 Analysis of Cap2 mutants and epitope-tagged CD-NTase and evidence that Cap2 conjugates the CD-NTase C-terminus to a target. (a) Efficiency of plating of the indicated phage when infecting E. coli expressing CBASS with the indicated genotype. Data plotted as in FIG. 1b. (b) Efficiency of plating of the indicated phage when infecting E. coli expressing CBASS with the indicated genotype. Data plotted as in FIG. 1b. (c) Western blot analysis of cell lysates from E. coli expressing CBASS with the indicated genotypes demonstrating that the mutations do not affect expression levels. (d) Western blot analysis of cell lysates from E. coli expressing CBASS with the indicated genotypes demonstrating that the mutations do not affect protein expression levels. (e) SDS-PAGE analysis of E. cloacae Cap2 activity assay. The indicated genotypes of His6-Cap2 and the CD-NTase were expressed from a single plasmid and the formation of a CD-NTase-His6-Cap2 conjugate was used as an indicator of Cap2 activity. (−): no CD-NTase; (+): wild-type CD-NTase; (AC): CD-NTase lacking its C-terminal 19 residues; C.D. Cap2: C548A/C109A. Blue asterisk indicates a putative intermediate with CD-NTase thioester-linked to the Cap2 E1 catalytic cysteine (C548). The formation of a CD-NTase-Cap2 conjugate in the absence of a functional E1 catalytic cysteine (C548A) indicates that in vitro, this residue is dispensable for catalysis and the nearby E2 catalytic cysteine (C109) can function in instead. (f) Cap2 E1 active site (yellow) in Cap2-CD-NTase cryoEM structure with the residues mutated in (a) indicated and the E1 active-site cysteine residue (C548 for Cap2) shown as a sphere and labeled. The CD-NTase C-terminus (orange) conjugated to AMP (black). (g) Left: SDS-PAGE analysis of Ni²⁺-purified E. cloacae His6-Cap2, expressed either alone or with full-length CD-NTase. Right: Protease treatment (TEV or Cap3) of the CD-NTase-His6-Cap2 conjugate. (h) Schematic of the inferred CD-NTase-His6-Cap2 conjugate formed upon coexpression of E. cloacae His6-Cap2 and CD-NTase, with cleavage sites for Cap3 and TEV protease indicated. (i) SDS-PAGE analysis with detection by coomassie (left) or αHA western blot (right) of αHA immunoprecipitated E. cloacae Cap2 coexpressed with HA-CD-NTase. C.D. Cap2: C548A/C109A. Red asterisk indicates band used for tryptic mass spectrometry analysis in (f-g). (j) Peptides detected in tryptic mass spectrometry of the marked band in (e), showing conjugation of CD-NTase to the N-terminus of a second HA-CD-NTase molecule. See Supplementary Table 7 for mass spectrometry data. (k) Collision-induced dissociation mass spectrum of the peptide indicated in (f), with b1 peak indicated (mass of 350.1533 is that of Met+(H+)+(Phe-Ala)). (l) SDS-PAGE analysis of Ni²⁺-purified E. cloacae His6-Cap2 with CD-NTase with the indicated genotype.

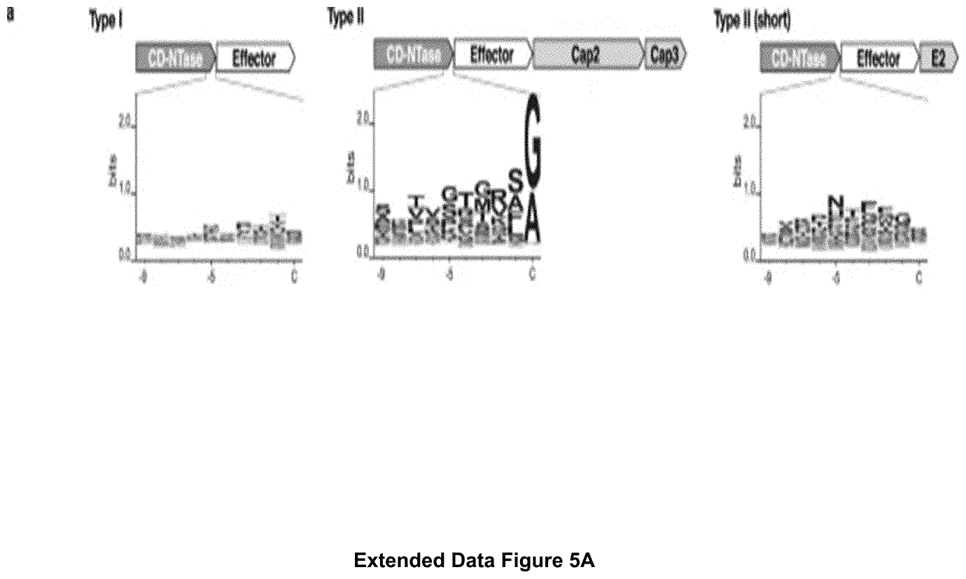

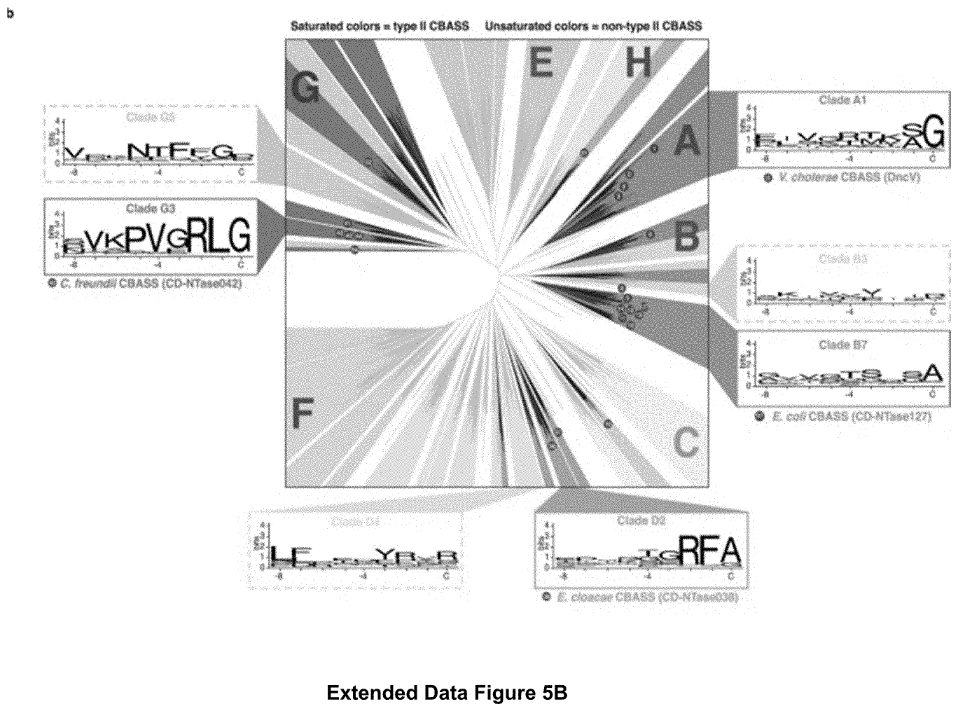

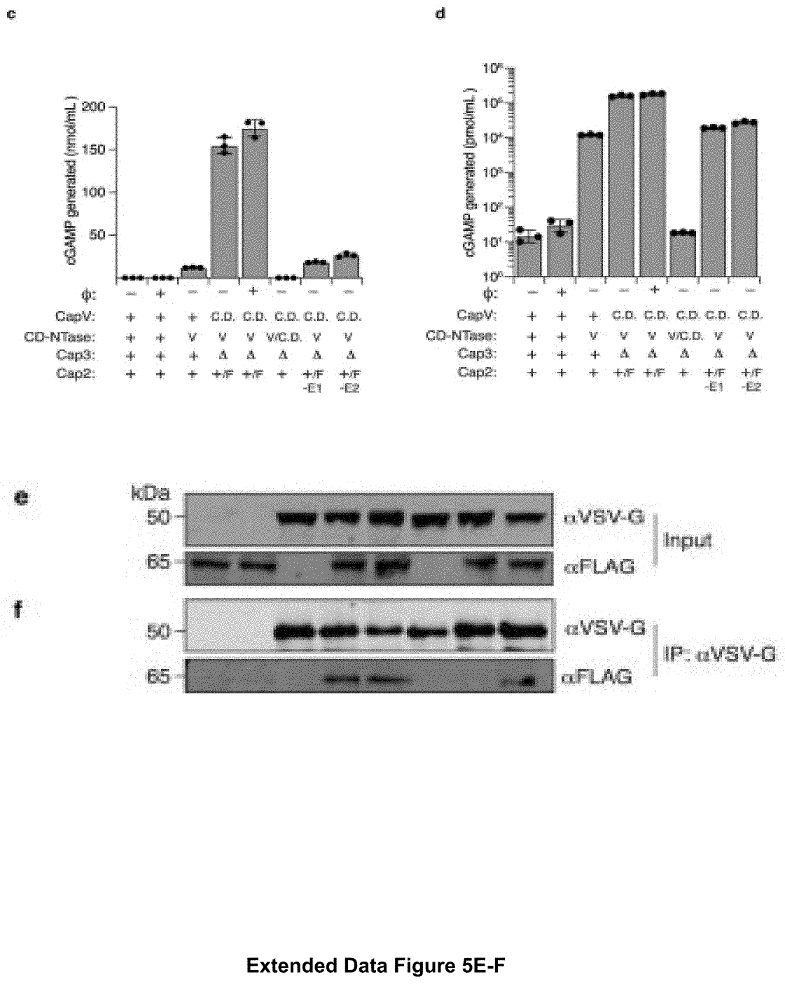

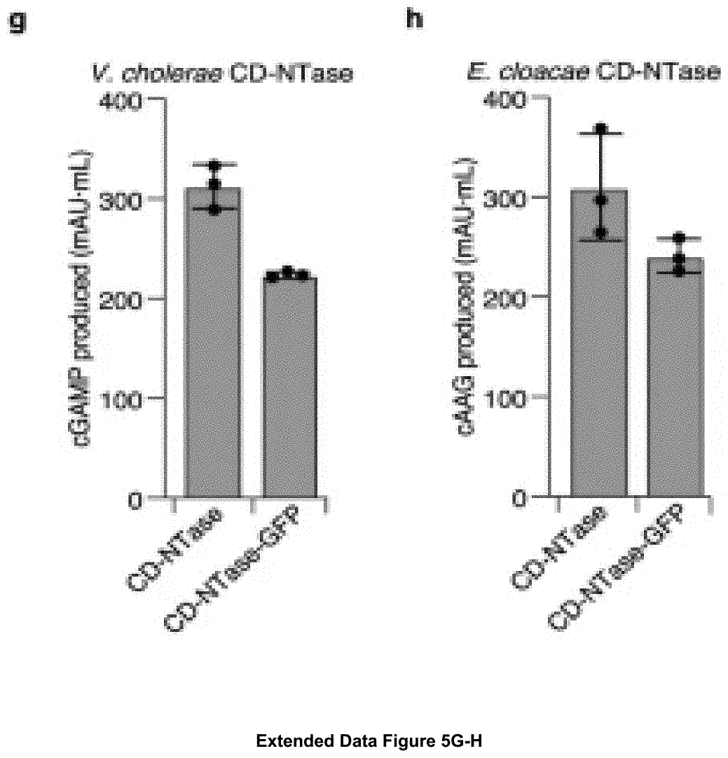

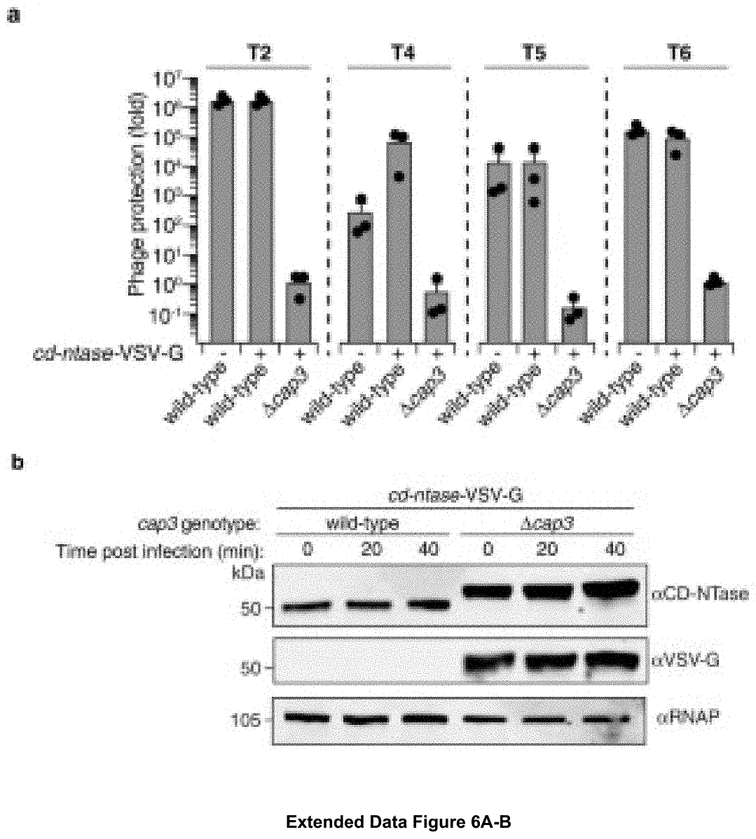

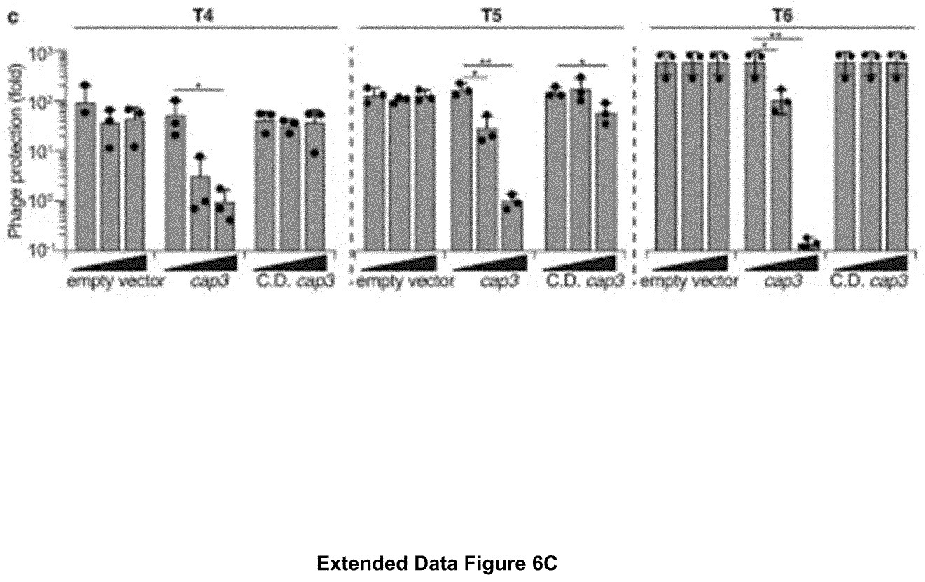

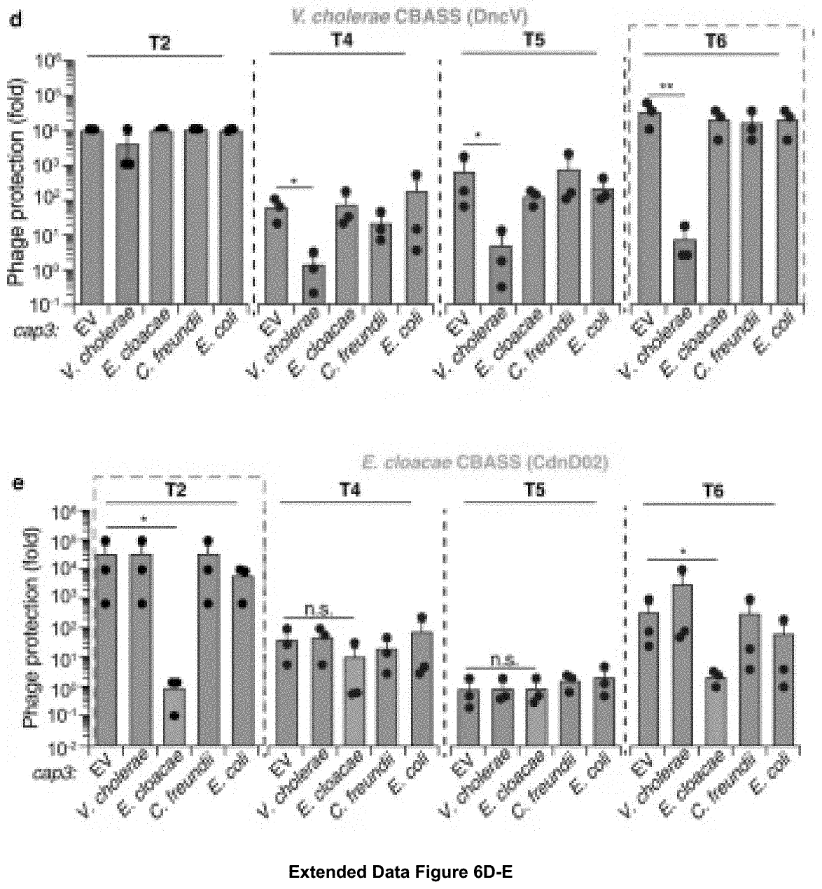

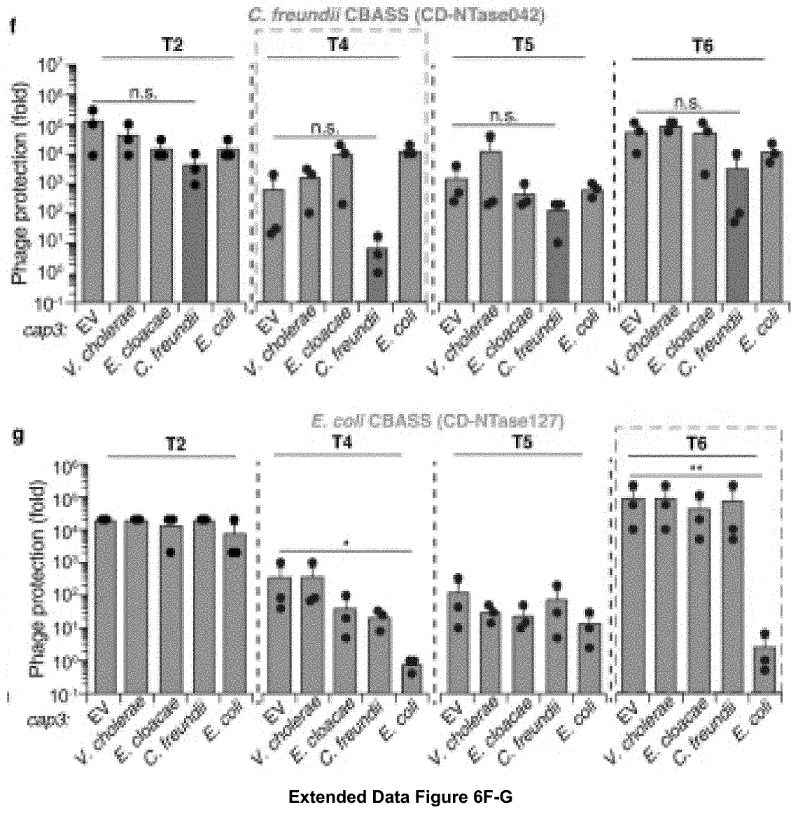

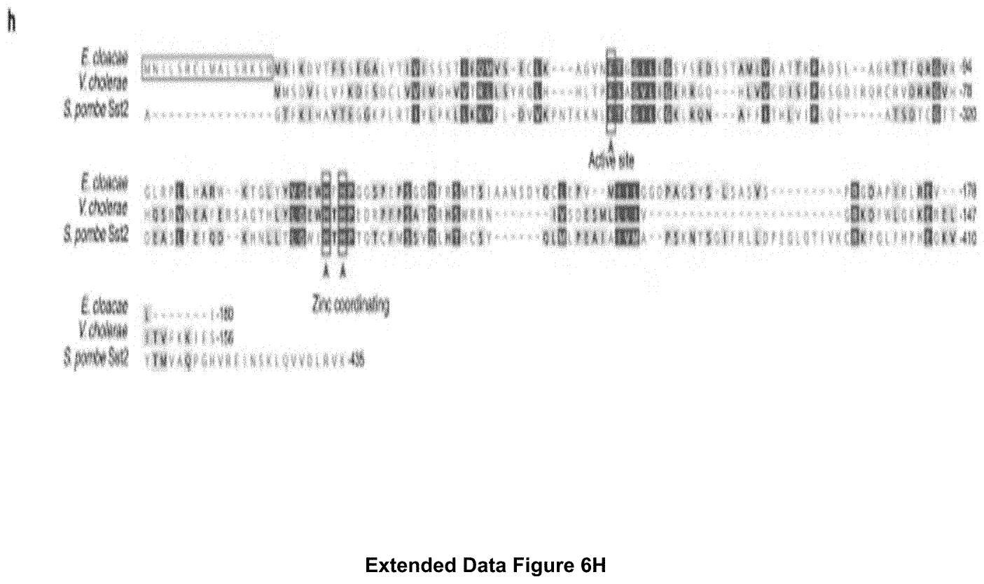

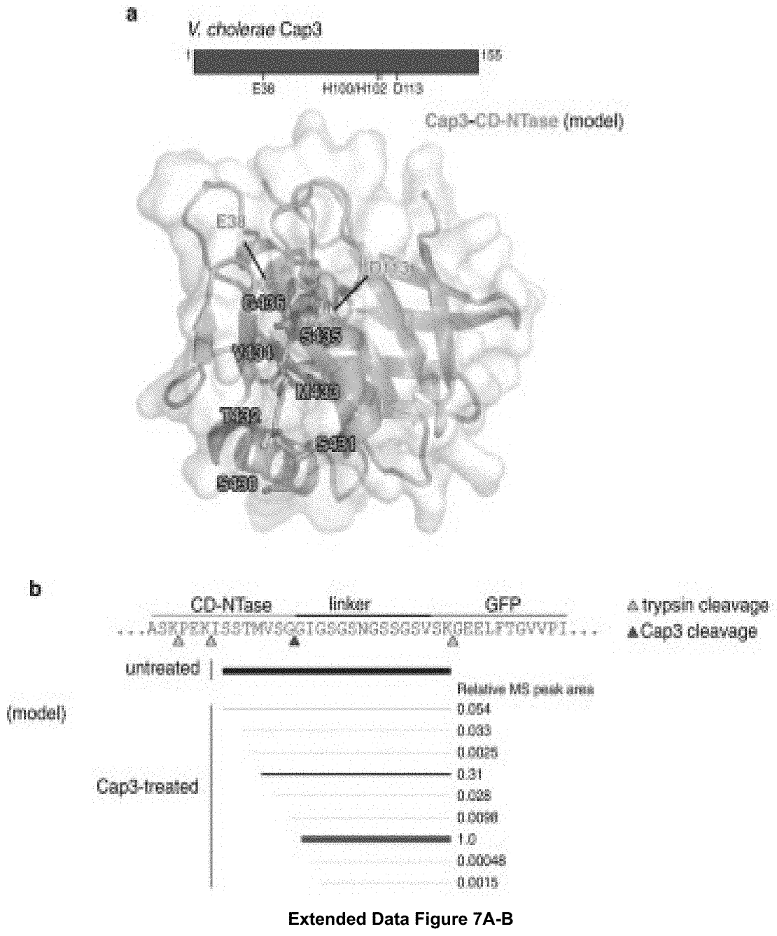

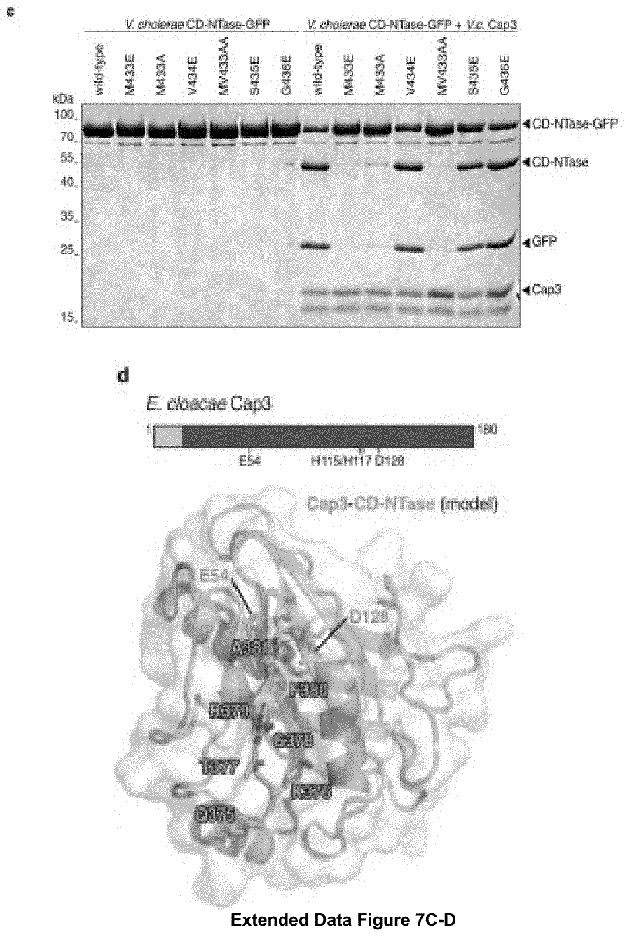

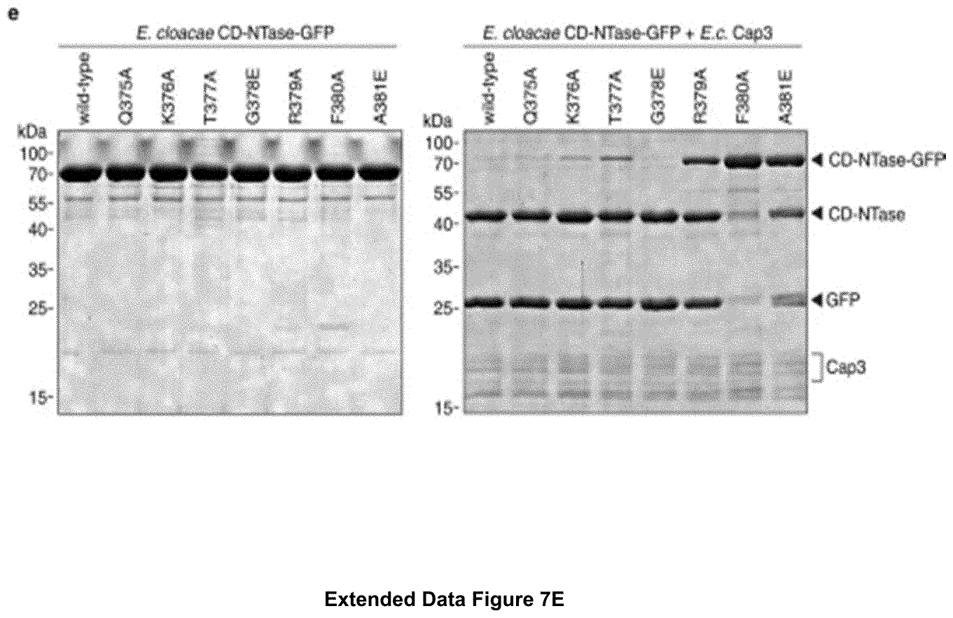

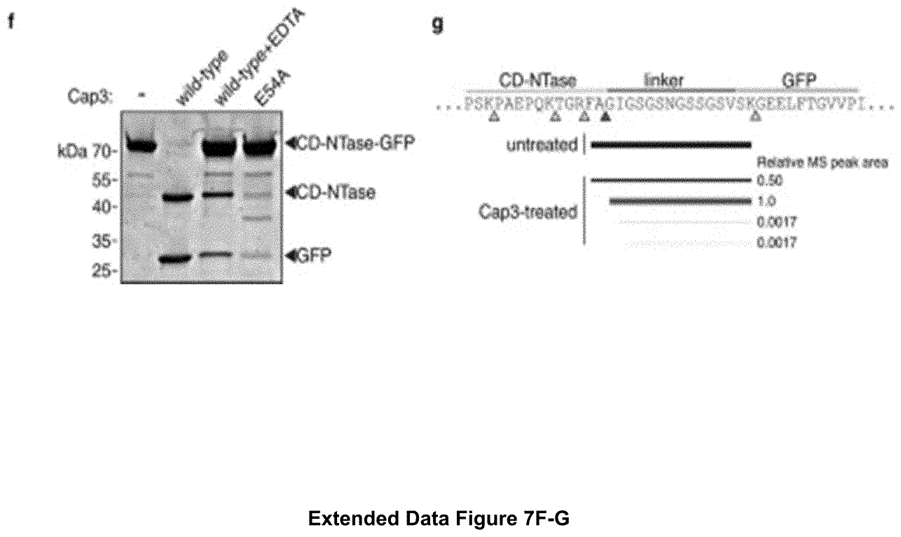

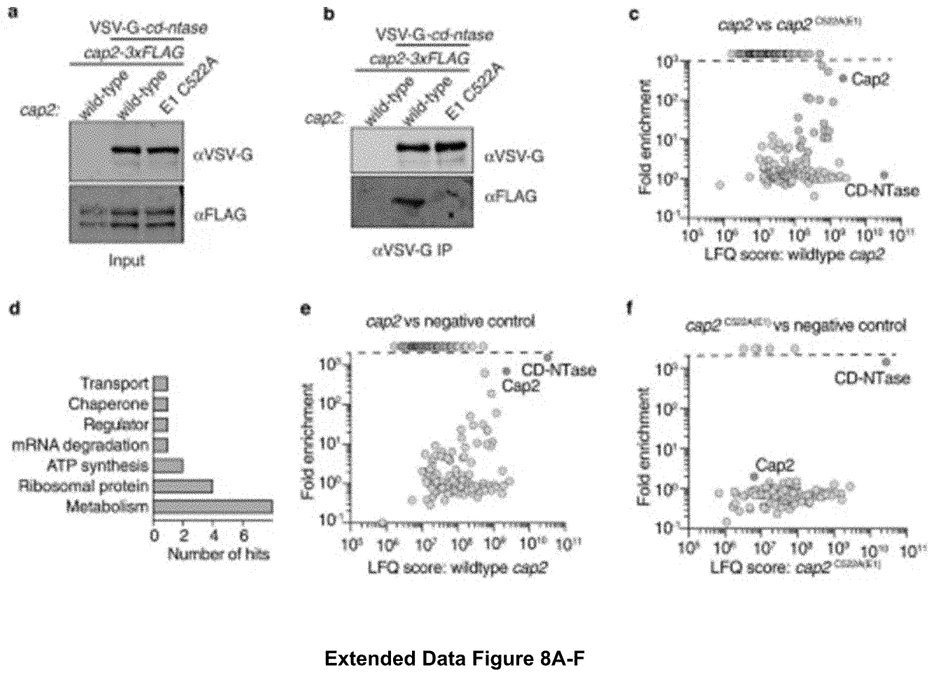

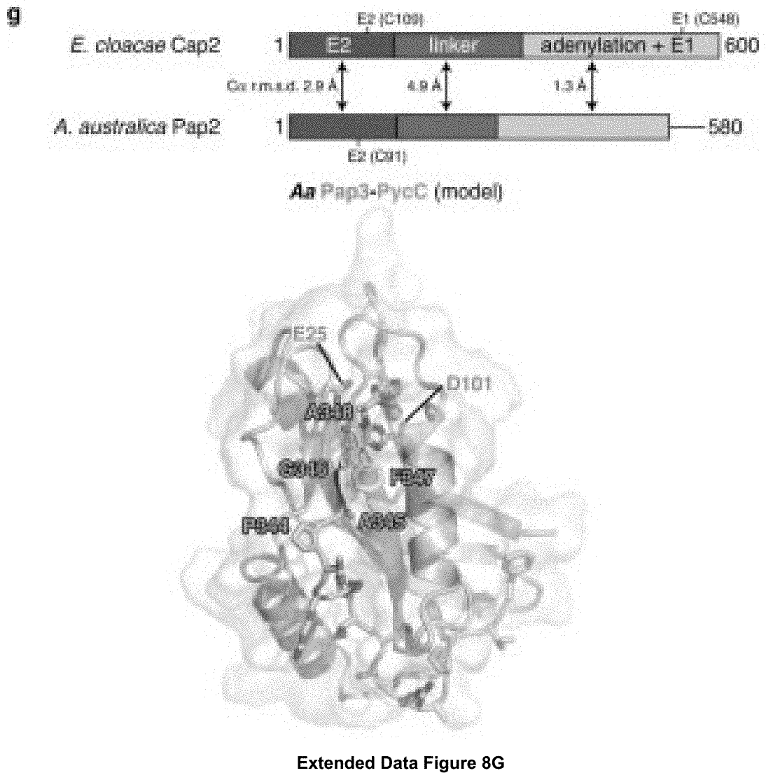

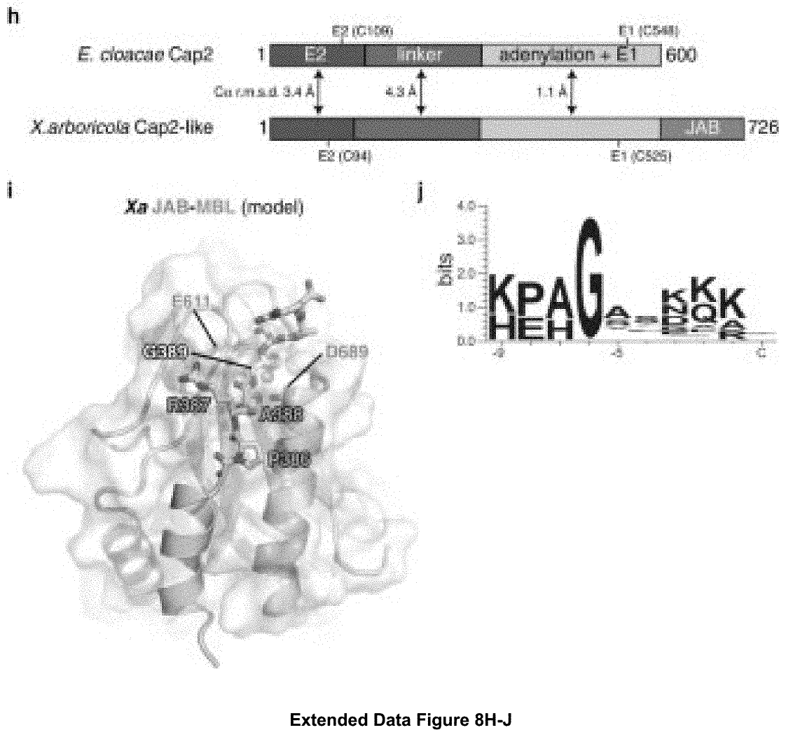

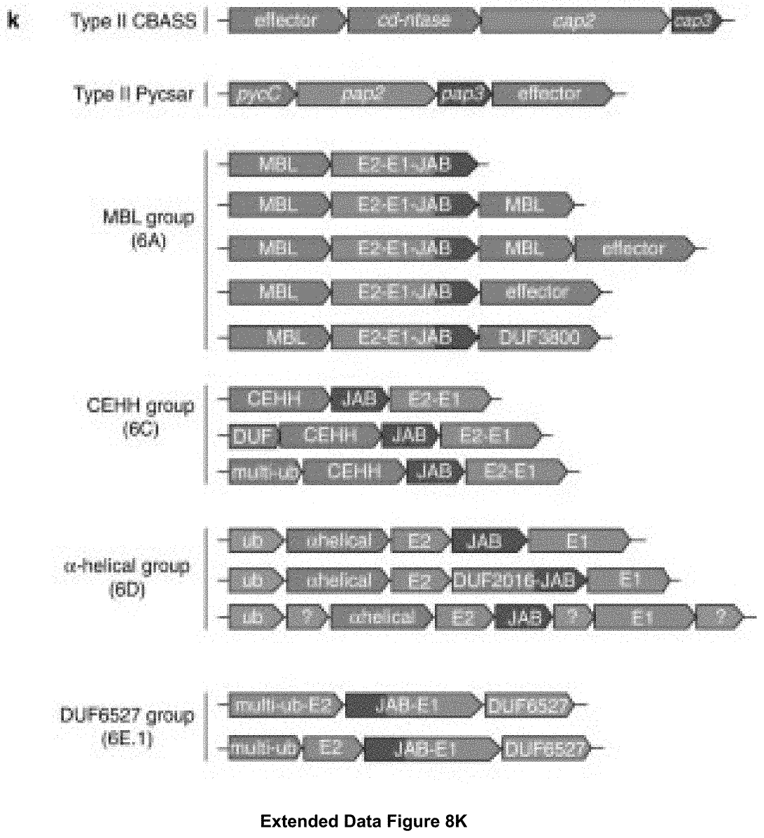

Extended Data FIG. 5 The C-terminus of the CD-NTase is conserved in type II CBASS systems and the quantification of CD-NTase-mediated second messenger generation. (a) Sequence logos of C-terminal 10 residues of the CD-NTase in type I (2284 sequences), type II (1556 sequences), and type II (short) (593 sequences) CBASS systems. Type II (short) CBASS systems encode an E2 ubiquitin transferase-like enzyme without a linked E1 domain, and do not encode a JAB isopeptidase. (b) Phylogenetic tree adapted from Whiteley et al., with sequence logos of the C-terminus for CD-NTase clades analyzed in Cap3 experiments (FIG. 4d, Extended Data FIG. 6d-g) shown. Saturated colors bordered with solid lines depict branches of the tree that contain type II systems, whereas the de-saturated colors bordered with dashed lines depict clades with non-type II systems. The CD-NTases used in this study are listed below each sequence logo. Blue circles with numbers represent CD-NTase numbers as reported previously. (c) cGAMP generated by αVSV-G immunoprecipitation from E. coli expressing CBASS operons with the indicated genotypes. Western blots of input are in (e) and immunoprecipitation in (f). N=3 technical replicates representative of three independent biological replicates. Data is presented as the mean values±the SEM. ϕ (−): no infection; ϕ (+): phage T2 at an MOI of 2; CapV (+): wild-type; CapV (C.D.): S62A; CD-NTase (+): wild-type; CD-NTase (C.D.): DID131AIA; CD-NTase (V): N-terminal VSV-G epitope tagged CD-NTase; Cap3 (+): wild-type; Cap3 (Δ): genetically deleted cap3; Cap2 (+): wild-type; Cap2 (F): C-terminal 3×-FLAG epitope tagged Cap2; Cap2 (E1): C522A; Cap2 (E2): C90A. (d) Data in (c) presented on a log₁₀ scale. (e) Whole cell western blot analysis of E. coli expressing CBASS with the indicated genotype. Data corresponds to the input for the immunoprecipitation shown in (a) and (b). (f) Western blot analysis of αVSV-G immunoprecipitation of E. coli expressing CBASS with the indicated genotypes. These data correspond to the samples used to measure cGAMP synthesis in (c) and (d). (g) Quantification of the cGAMP produced by the V. cholerae CD-NTase with, or without, a C-terminal GFP fusion. (h) Quantification of the cAAG produced by the E. cloacae CD-NTase with, or without, a C-terminal GFP fusion. For (g) and (h) N=3 independent biological replicates and the data is presented as the mean±the SD.

Extended Data FIG. 6 Cap3 overexpression inhibits phage protection by cognate CBASS. (a) Efficiency of plating of the indicated phage when infecting E. coli expressing CBASS with the indicated genotype. cd-ntase-VSV-G (−): wild-type CD-NTase; cd-ntase-VSV-G (+): C-terminal VSV-G epitope tagged CD-NTase; wild-type indicates otherwise a full CBASS operon; Δcap3: CBASS operon with only cap3 deletion. Data plotted as in FIG. 1b. (b) Western blot analysis of cell lysates from E. coli expressing CBASS with the indicated genotypes, abbreviated as in (a). Cells were infected with phage T5 at an MOI of 2 for the indicated time prior to harvesting for analysis. (c) Efficiency of plating of the indicated phage when infecting E. coli expressing CBASS Δcap3 in the absence or presence of overexpressed cap3 with the indicated genotype. Data plotted as in FIG. 4a. C.D. cap3: HTH101ATA. A Two-sided Student's t-test was used to calculate significance; n.s., p>0.05; *, p<0.05; **, p<0.001. See Extended Data FIG. 6h for protein alignment. (d) Efficiency of plating of the indicated phage when infecting E. coli expressing a full CBASS operon from V. cholerae in the absence or presence of overexpressed cap3 from another CBASS system, indicated on the x-axis. Data plotted as in FIG. 4a. (e) Efficiency of plating of the indicated phage when infecting E. coli expressing a full CBASS operon from E. cloacae in the absence or presence of overexpressed cap3 from another CBASS system, indicated on the x-axis. Data plotted as in FIG. 4a. (f) Efficiency of plating of the indicated phage when infecting E. coli expressing a full CBASS operon from C. freundii in the absence or presence of overexpressed cap3 from another CBASS system, indicated on the x-axis. Data plotted as in FIG. 4a. (g) Efficiency of plating of the indicated phage when infecting E. coli expressing a full CBASS operon from E. coli in the absence or presence of overexpressed cap3 from another CBASS system, indicated on the x-axis. Data plotted as in FIG. 4a. For (d-g) the red dashed boxes indicated the data utilized in FIG. 4d, See Supplementary Table 5 for relevant accession numbers. (h) Protein alignment of the JAMM/JAB protease Sst2 from S. pombe55 (Uniprot ID Q9P371; residues 235-435), Cap3 from E. cloacae, and Cap3 from V. cholerae. The active site glutamate, as well as two zinc-coordinating histidine residues, are noted. For experiments using Cap3 from E. cloacae, the first 16 annotated amino acids (green box) were removed as Applicants found the translation start site is likely misannotated for this gene. See Supplementary Table 5 for relevant accession numbers.

Extended Data FIG. 7 Cap3 cleavage of a CD-NTase model substrate. (a) Domain schematic and predicted structure/model of the V. cholerae Cap3-CD-NTase complex with the CD-NTase C-terminus and Zn²⁺ ion manually modeled from an overlay with a structure of S. pombe Sst2 bound to ubiquitin75 (PDB ID 4K1R). (b) Summary of tryptic digest mass spectrometry analysis of the V. cholerae Cap3-treated CD-NTase bands as in FIG. 4b. Pink arrow indicates the inferred Cap3 cleavage site; gray arrows indicate trypsin cleavage sites. See Supplementary Table 3 for data. (c) Coomassie stained SDS-PAGE of a V. cholerae model substrate (CD-NTase-GFP fusion protein) with the indicated mutations in the CD-NTase C-terminus, with and without incubation with V. cholerae Cap3. (d) Domain schematic and predicted structure/model of the E. cloacae Cap3-CD-NTase complex with the CD-NTase C-terminus and Zn²⁺ ion manually modeled from an overlay with a structure of S. pombe Sst2 bound to ubiquitin (PDB ID 4K1R). (e) Coomassie stained SDS-PAGE of an E. cloacae model substrate (CD-NTase-GFP fusion protein) with the indicated mutations in the CD-NTase C-terminus, with and without incubation with E. cloacae Cap3. (f) Coomassie stained SDS-PAGE of an E. cloacae model substrate (CD-NTase-GFP fusion protein) incubated with E. cloacae Cap3 with the indicated reaction condition/genotype. (g) Summary of tryptic digest mass spectrometry analysis of the E. cloacae Cap3-treated CD-NTase bands as in (f), showing the putative Cap3 cleavage site. Pink arrow indicates the inferred Cap3 cleavage site; gray arrows indicate trypsin cleavage sites. See Supplementary Table 3 for data.

Extended Data FIG. 8 CD-NTase immunoprecipitation reveals numerous potential protein targets and Cap2 homologs are found in other bacteria. (a) Western blot analysis of cell lysates generated from E. coli expressing CBASS Δcap3 with the additional indicated genotypes. (b) Western blot analysis of αVSV-G immunoprecipitations generated from E. coli expressing CBASS Δcap3 with the additional indicated genotypes. These samples were used in the mass spectrometry analysis displayed in (c-f). (c) Mass spectrometry of immunoprecipitated VSV-G-CD-NTase as shown in (b). Data are label free quantitation (LFQ) score and fold enrichment comparing immunoprecipitations from bacteria expressing CBASS Δcap3 to a strain expressing CBASS Δcap3 cap2^{C522A(E1)}. Both strains encode an N-terminally VSV-G tagged CD-NTase. Cap2 and CD-NTase are represented as colored circles corresponding to FIG. 1a and are labeled. Proteins which Applicants determined were significantly enriched (LFQ>10⁸ and a fold enrichment >4) are colored in pink. Circles above the dotted line are proteins with peptides identified only in the sample listed on the x-axis. See Supplementary Table 8 for data. (d) Characterization of the predicted functions of the proteins that were significantly enriched in (c). (e) Mass spectrometry of immunoprecipitated VSV-G-CD-NTase as in (c) comparing immunoprecipitations from bacteria expressing CBASS Δcap3 where CD-NTase has an N-terminal VSV-G tag to a strain expressing CBASS lacking a VSV-G tag (negative control). (f) Mass spectrometry of immunoprecipitated VSV-G-CD-NTase as in (c) comparing immunoprecipitations from bacteria expressing CBASS Δcap3 cap2^{C522A(E1)} where CD-NTase has an N-terminal VSV-G tag to a strain expressing CBASS lacking a VSV-G tag (negative control). (g) Top: Structural comparison between E. cloacae Cap2 and a predicted structure/model of Azohydromonas australica Pap2. Cα r.m.s.d. values are reported for superposition of individual domains: E2 domain (52 Cα atoms overlaid), linker (90 Cα atoms), and E1 (133 Cα atoms). Predicted catalytic cysteine residues are noted for each protein. Bottom: Structural prediction of a Pap3 (pink) and the C-terminus of PycC (yellow) from A. australica. Predicted active site residues of Pap3 are shown as sticks with a zinc ion (gray) modeled from a structure of S. pombe Sst2 bound to ubiquitin (PDB ID 4K1R). (h) Structural comparison between E. cloacae Cap2 and a predicted structure/model of the Cap2-like protein from the Xanthomonas arboricola MBL-group operon. Cα r.m.s.d. values are reported for superposition of individual domains: E2 domain (94 Cα atoms overlaid), linker (50 Cα atoms), and E1 (154 Cα atoms). Predicted catalytic cysteine residues are noted for each protein. (i) Predicted structure/model of a complex between X. arboricola JAB domain (pink) and the C-terminus of MBL (yellow). Predicted active site residues of the JAB domain are shown in sticks, with a zinc ion (gray) modeled from a structure of S. pombe Sst2 bound to ubiquitin75 (PDB ID 4K1R). The conserved glycine residue of MBL (white) is positioned for cleavage. (j) Sequence logo for the C-terminal 9 residues of 268 MBL encoded within MBL-group operons. See also Supplementary Table 9. (k) Operon structure of previously described and proposed phage defense systems that contain E1, E2 and JAB domain containing proteins along with operons of unknown function that contain these domains. Operons are grouped by conserved protein domains. The E1-superfamily these groups is also indicated in paratheses. Genes are colored by domain type; E1 and E2 domains, blue; JAB domains, purple; all other domains, grey. Metallo-β-lactamase (MBL); metal binding domain (CEHH); tandem β-grasp fold domain containing protein (multi-ub); single β-grasp fold domain containing protein (ub); Domains of unknown function (DUF); genes with no discernable domains (?). See also Supplementary Table 9.

### DETAILED DESCRIPTION OF THE INVENTION

The invention describes the use of Cap2 to generate protein fusions bona fide peptide bond between two input proteins with minimal “scar” remnants, enabling much higher flexibility in its use compared to existing systems which use “split inteins.” Such traditional fusion systems involve the generation of two fusion proteins that include the two distinct halves of the intein system. After reaction of these two halves, the two proteins are fused but with a multiple amino acid “scar” left over from the intein halves. The invention further describes the use of Cap3 to cleave peptides in a sequence specific manner. This advancements allow for eh generation of a series of Cap3-mediate truncation or simply offer additional cleavage sequences.

The present invention further includes system, methods, and compositions to generate a fusion peptide. In one preferred embodiment, an exemplary Cap2 enzyme of the invention, or a fragment or variant thereof, may be engineered to include a target recognition motif. This target recognition motif of the invention may include an antibody, or antibody fragment thereof, configured to bind to and couple a target peptide. In alternative embodiments, target recognition motif of the invention may include an engineered protein binding motif configured to bind to and couple a target peptide. Examples of engineered antibodies or peptides that can recognize and bind to a target recognition motif, as well as the rational design and implementation of such structural motifs may be generally described, for example by Trier N., et al., Peptides, Antibodies, Peptide Antibodies and More. Int J Mol Sci. 2019; 20(24):6289. Published 2019 Dec. 13.

Again, in a preferred embodiment an exemplary Cap2 enzyme of the invention, or a fragment or variant thereof, may be a fusion peptide having a first domain of a Cap2 enzyme, coupled, for example through a covalent or peptide bond to a second domain comprising a target recognition motif, such as an antibody or designed peptide motif.

In another embodiment of the invention, one or more target peptides may recognize and be coupled with the Cap2 enzyme through the target recognition motif. The target peptide of the invention may be expressed in an in vivo system, and may be endogenous or heterologous to that system. In a preferred embodiment, such in vivo system may include a cell, cell-based assay, tissue, or subject, and preferably a human subject. The target peptide of the invention may preferably be expressed in an in vivo system, such as a bacterial, algal, yeast, or eukaryotic-based protein production system, and further isolated and or purified so as to be applicable in an in vitro system. As noted above, the target peptide of the invention may be endogenous or heterologous to the bacterial, algal, yeast, or cell-based protein expression system. The target peptide of the invention may further be a wild-type, or engineered peptide. In a preferred embodiment, the target peptide may include a metabolically relevant peptide, the activity of which may be inhibited or increased through the binding of a first peptide as described below. In alternative embodiments, the target peptide may include a metabolically relevant peptide, the activity of which may be increased through the binding of a first peptide as described below.

The first peptide of the invention may be expressed in an in vivo system, and may be endogenous or heterologous to that system. In a preferred embodiment, such in vivo system may include a cell, cell-based assay, tissue, or subject, and preferably a human subject. The first peptide of the invention may preferably be expressed in an in vivo system, such as a bacterial, algal, yeast, or eukaryotic-based protein production system, and further isolated and or purified so as to be applicable in an in vitro system. As noted above, the first peptide of the invention may be endogenous or heterologous to the bacterial, algal, yeast, or cell-based protein expression system. The first peptide of the invention may further be a wild-type, or engineered peptide. In a preferred embodiment, the first peptide may include a metabolically relevant peptide, the activity of which may be inhibited through the binding of a target peptide as described below. In alternative embodiments, the first peptide may the first peptide may include a metabolically relevant peptide, the activity of which may be increased through the binding of a target peptide as described below.

As noted previously, in one embodiment of the invention a Cap2 enzyme may facilitate the fusion of a target peptide and a first peptide in an in vivo or in vitro system such that the activity, localization or other characteristic of the target peptide or first peptide is inhibited or increased, or other modified. For example, in one embodiment, a target peptide may include a metabolically relevant peptide related to one or more cellular processes, and preferably one or more cellular processes that are related to a disease or condition in humans. In this embodiment, an exemplary Cap2 enzyme may facilitate the fusion of a metabolically relevant target peptide and a first peptide wherein the resulting fusion peptide preserves the original sequences of the target and first peptides. Moreover, in this embodiment, the first peptide may modulate the activity of the target peptide, such as by inhibit its enzymatic activity, for example through steric interference, or inducing a conformation change or cleavage of the target peptide. In alternative embodiments, the first peptide may modulate the activity of the target peptide, such as by increasing its enzymatic activity, for example through inducing a conformation changes or adding additional catalytic or binding motifs.

In other embodiment, the first peptide may include a tag that may allow identification, isolation or purification of the target peptide. In still further embodiments, the tag may further be coupled with, or configured to be coupled with a another peptide or chemical composition, such as a therapeutic compound that modulates the activity of the target peptide. The first peptide may further modulate the localization of the target peptide in an in vivo system. For example, the first peptide may include a localization or targeting signal peptide causing the target peptide to be localized to a different location in a cell, or an export or import signal peptide causing the target peptide to be expelled, or brought into a cell further modulating its activity or availability to, for example a substrate or receptor. In still further embodiment, the first peptide may include a peptide signaling that initiates the destruction or degradation of the target peptide.

In a preferred embodiment, a Cap2 enzyme, a first peptide, and a target peptide may form a complex of said first peptide and said target peptide are coupled with a Cap2 enzyme. In this configuration, the Cap2 enzyme ligates the first peptide to and target peptide prior to disengaging from the complex, preferably forming a scar-free fusion peptide, which preserves the amino acid sequences of the peptides.

In a preferred embodiment, a Cap2 enzyme, a first peptide, and a target peptide may form a complex with and intermediary peptide coupling said first peptide with said Cap2 enzyme. In this configuration, the intermediary peptide forms part of the complex being coupled with the Cap2 enzyme and the first peptide through an intermediary peptide recognition motif. In a preferred embodiment, intermediary peptide comprises a CD-Ntase, sometime referred to in the parent application as a cGAS peptide, or a fragment or variant having an endogenous or engineered intermediary peptide recognition motif that recognizes and binds to a this CD-NTase recognition motif, sometimes sometime referred to in the parent application as a cGAS recognition motif.

As outline in the schematic below, a Cap2 enzyme according to SEQ ID NO's 1-2, 13 or 16 or a fragment or variant thereof, a first peptide, and a target peptide may form a complex with and intermediary CD-NTase peptide according to SEQ ID NO's. 5-6, 12 or 15, or a fragment or variant thereof, coupling the first peptide with said Cap2 enzyme. In this configuration, the intermediary CD-NTase peptide forms part of the complex being coupled with the Cap2 enzyme, which may be a homo-dimer, and the first peptide through an intermediary peptide recognition motif. In a preferred embodiment, intermediary peptide comprises a CD-NTase peptide, or a fragment or variant having an endogenous or engineered intermediary peptide recognition motif that recognizes and binds to a this CD-NTase recognition motif.

### Exemplary Cap2 Complex

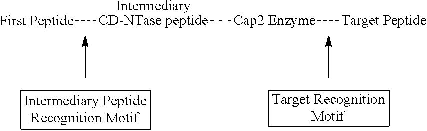

The invention may include isolated nucleotide sequence, as well expression vectors, a nucleotide sequence, operably linked to a promotor, encoding a peptide fusion system. In this embodiment, an expression vector, such as a plasmid or other similar vector known in the art, may be engineered to include one or more nucleotide sequences, operably linked to a regulatory sequence, such as promoter. In this preferred embodiment, the one or more nucleotide sequences may encode one or more of the following: 1) a first peptide, and a Cap2 enzyme having a target recognition motif, and optionally a target peptide; and optionally an intermediary peptide. The first peptide encoded in the expression vector may include an intermediary peptide recognition motif, an in particular a CD-NTase recognition motif that is configured to recognize and bind to an intermediary peptide, which may preferably include a CD-NTase peptide, or a fragment or variant thereof, and preferably a CD-NTase peptide selected from SEQ ID NO's. 5-6, 12 or 15, or a fragment or variant thereof.

In this embodiment, the expression vector of the invention may be used to transform, and be expressed in a cell, and preferably mammalian cell such as a human cell. In alternative embodiment, the expression vector of the invention may be expressed in an in vitro system, such as a peptide production system, or a cell-free transcription/translation express system or other assay.

In another embodiment, the invention may include novel, systems, methods and compositions for cleaving a peptide. In one preferred embodiment, the invention may include the step of establishing a complex of a CD-NTase peptide coupled with a target conjugate, which may preferably include a peptide, and more preferably include an engineered target peptide to be cleaved. This complex may be contacted with a Cap3 enzyme, or a fragment or variant thereof, that wherein catalyzes the cleavage of the CD-NTase peptide from the target conjugate.

In one specific preferred embodiment, the invention may include the step of establishing a complex of a CD-NTase peptide according to SEQ ID NO's. 5-6, 12 or 15, or a fragment or variant thereof, and a target conjugate, which may preferably include a peptide, and more preferably include an engineered target peptide to be cleaved. In a preferred embodiment, a complex of the invention may be contacted with a Cap3 enzyme according to SEQ ID NO's. 3-4, 14 or 17, or a fragment or variant thereof, the catalyzes the hydrolytic cleavage after the C-terminal residue of the CD-NTase peptide.

In another preferred embodiment, a complex of the invention may be contacted with a Cap3 enzyme according to SEQ ID NO's. 3-4, 14 or 17, or a fragment or variant thereof, wherein cleavage of said CD-NTase peptide from said target conjugate occurs at a cleavage motif in a sequence specific-manner. In this embodiment, the cleavage motif of the invention may include an amino acid sequence selected from the group consisting of: SEQ ID NO's. 7-8, or 19-20 as shown below:

SEQ ID NO. 7: Cap3 - cleavage site
(CD-Ntase Vibrio cholerae (SEQ ID NO. 6))
ISSTMVSG^{∧}X
SEQ ID NO. 8: Cap3 - cleavage site
(CD-Ntase Enterobacter cloacae (SEQ ID NO. 5))
PQKTGRFA^{∧}X
SEQ ID NO. 19: Cap 3 - cleavage site
(CD-Ntase Citrobacter freundii (SEQ ID NO. 12))
VAPVGRLG^{∧}X
SEQ ID NO. 20: Cap3 - cleavage site
(CD-Ntase E. coli (SEQ ID NO. 18))
VVPAGRSA^{∧}X

wherein \hat{ } is a cleavage site and X is an amino acid residue of a target conjugate

In another embodiment, the invention include an expression vector having a nucleotide sequence, operably linked to a promotor, encoding one or more components of a peptide cleavage system. In this embodiment, the expression vector of the invention may include an expression vector having a nucleotide sequence, operably linked to a promotor, encoding a Cap3 enzyme, or a fragment or variant thereof, and optionally a CD-NTase peptide, and/or a target conjugate, or a combination of the same.

In a preferred specific embodiment, the expression vector of the invention may include an expression vector having a nucleotide sequence, operably linked to a promotor, encoding a Cap3 enzyme according to SEQ ID NO's. 3-4, 14 or 17, or a fragment or variant thereof, and optionally a CD-NTase peptide according to SEQ ID NO. 5-6, 12 or 15, or a fragment or variant thereof, and/or a target conjugate, which may preferably be an engineered peptide, or a combination of the same, wherein said Cap3 enzyme catalyzes the hydrolysis of CD-NTase and the target conjugate in an in vitro, or in vivo system in a sequence specific manner as described herein.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

As used herein the singular forms “a”, “an”, and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including”, as well as other related forms, such as “includes” and “included”, is not limiting.

The term “about” as used herein is a flexible word with a meaning similar to “approximately” or “nearly”. The term “about” indicates that exactitude is not claimed, but rather a contemplated variation. Thus, as used herein, the term “about” means within 1 or 2 standard deviations from the specifically recited value, or ±a range of up to 20%, up to 15%, up to 10%, up to 5%, or up to 4%, 3%, 2%, or 1% compared to the specifically recited value.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “isolated,” when applied to a nucleic acid or protein, denotes that the nucleic acid or protein is essentially free of other cellular components with which it is associated in the natural state. It is preferably in a homogeneous state although it can be in either a dry or aqueous solution. Purity and homogeneity are typically determined using analytical chemistry techniques such as polyacrylamide gel electrophoresis or high performance liquid chromatography. A protein which is the predominant species present in a preparation is substantially purified. In particular, an isolated gene is separated from open reading frames which flank the gene and encode a protein other than the gene of interest. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The term “coupled” or “ligated” when applied to a peptide of the invention may include direct chemical bonds, such as covalent linkages, such as through the generation of fusion or chimera proteins. In alternative embodiment, couple term “coupled” when applied to a peptide of the invention may include instances where a peptide of the invention may be bound to another peptide or molecule through an intermediary compound or molecule, such as a peptide.

The terms “conjugating” or “linking” or “coupling” in the context of the present invention with respect to connecting two or more molecules or components to form a complex refers to joining or conjugating said molecules or components, e.g. proteins, via a covalent bond, particularly an isopeptide bond which forms between the peptides that may be mediated by a Cap2 enzyme.

A “domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “engineered” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The term “peptide tag” or “peptide linker” as used herein generally refers to a peptide or oligopeptide. There is no standard definition regarding the size boundaries between what is meant by peptide or oligopeptide but typically a peptide may be viewed as comprising between 2-20 amino acids and oligopeptide between 21-39 amino acids. Accordingly, a polypeptide may be viewed as comprising at least 40 amino acids, preferably at least 50, 60, 70 or 80 amino acids. Thus, a peptide tag or linker as defined herein may be viewed as comprising at least 12 amino acids, e.g. 12-39 amino acids, such as e.g. 13-35, 14-34, 15-33, 16-31, 17-30 amino acids in length, e.g. it may comprise or consist of 12, 13, 14, 15, 16, 17, 18, 19, 20, 21, 22 or 23 amino acids.

A “fusion” or “chimera” protein is a polypeptide produced when two heterologous nucleotide sequences or fragments thereof coding for two (or more) different polypeptides not found fused together in nature are fused together in the correct translational reading frame.

As used herein, a “functional” polypeptide or “functional fragment” is one that substantially retains at least one biological activity normally associated with that polypeptide (e.g., nucleosome formation).

In particular embodiments, the “functional” polypeptide or “fragment” substantially retains all of the activities possessed by the unmodified peptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide).

The term, “expression” or “expressing” refers to production of a functional product, such as, the generation of an RNA transcript from an introduced construct, an endogenous DNA sequence, or a stably incorporated heterologous DNA sequence. A nucleotide encoding sequence may comprise intervening sequence (e.g., intrans) or may lack such intervening non-translated sequences (e.g., as in cDNA). Expressed genes include those that are transcribed into mRNA and then translated into protein and those that are transcribed into RNA but not translated (for example, siRNA, transfer RNA, and ribosomal RNA). The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

An “expression vector” or “vector” refers to a nucleic acid construct, which when introduced into a host cell, results in transcription and/or translation of a RNA or polypeptide, respectively. More specifically, the term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria. Again, more specifically, “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.”

In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

A “variant,” or “isoform,” or “protein variant” is a member of a set of similar proteins that perform the same or similar biological roles. For example, fragments and variants of the disclosed polynucleotides and amino acid sequences of the invention encoded thereby are also encompassed by the present invention. By “fragment” is intended a portion of the polynucleotide or a portion of the amino acid sequence. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide.

As used herein, a “native” or “wildtype” polynucleotide or polypeptide comprises a naturally occurring nucleotide sequence or amino acid sequence, respectively. In one embodiment, a “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., at least 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention. In some embodiments, such fragments can comprise, consist essentially of, and/or consist of peptides having a length of less than about 4, 6, 8, 10, 12, 15, 20, 25, 30, 35, 40, 45, 50, 75, 100, 150, or 200 consecutive amino acids of a polypeptide or amino acid sequence according to the invention.

The term “fragment,” as applied to a polynucleotide, can further be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., at least 90%, 92%, 95%, 98%, 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention. In some embodiments, such fragments can comprise, consist essentially of, and/or consist of oligonucleotides having a length of less than about 8, 10, 12, 15, 20, 25, 30, 35, 40, 45, 50, 75, 100, 150, or 200 consecutive nucleotides of a nucleic acid or nucleotide sequence according to the invention.

The term “gene” or “nucleotide sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “gene” or “nucleotide sequence” as used herein can mean a DNA sequence that is transcribed into mRNA which is then translated into a sequence of amino acids characteristic of a specific polypeptide.

The term “heterologous” refers to a nucleic acid fragment or protein that is foreign to its surroundings. In the context of a nucleic acid fragment, this is typically accomplished by introducing such fragment, derived from one source, into a different host. Heterologous nucleic acid fragments, such as coding sequences that have been inserted into a host organism, are not normally found in the genetic complement of the host organism. As used herein, the term “heterologous” also refers to a nucleic acid fragment derived from the same organism, but which is located in a different, e.g., non-native, location within the genome of this organism. A nucleic acid fragment that is heterologous with respect to an organism into which it has been inserted or transferred is sometimes referred to as a “transgene.”

The term “endogenous” refers to a component naturally found in an environment, i.e., a gene, nucleic acid, miRNA, protein, cell, or other natural component expressed in the subject, as distinguished from an introduced component, i.e., an “exogenous” component.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art and is understood as included in embodiments where it would be appropriate. Nucleotides may be referred to by their commonly accepted single-letter codes. Unless otherwise indicated, amino acid sequences are written left to right in amino to carboxyl orientation, respectively. Amino acids may be referred to herein by either their commonly known three letter symbols or by the one-letter symbols as generally understood by those skilled in the relevant art.

“Operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence.

The term “promoter” or “regulatory element” refers to a region or nucleic acid sequence located upstream or downstream from the start of transcription and which is involved in recognition and binding of RNA polymerase and/or other proteins to initiate transcription of RNA. Promoters useful in the present methods include, for example, constitutive, strong, weak, tissue-specific, cell-type specific, seed-specific, inducible, repressible, and developmentally regulated promoters.

As used herein, the term “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into, such as a bacterium.

The term “antibody”, as used herein, refers to an immunoglobulin, e.g., an antibody, and to antigen binding portions thereof, e.g., molecules that contain an antigen binding site which specifically binds an antigen, such as a polypeptide. A molecule which specifically binds to a given polypeptide, but does not substantially bind other molecules in a sample, e.g., a biological sample, which naturally contains the polypeptide. Antibody molecules include “antibody fragments” which refers to a portion of an intact antibody that is sufficient to confer recognition and specific binding to a target antigen. Examples of antibody fragments include, but are not limited to, Fab, Fab′, F(ab′)2, and Fv fragments, linear antibodies, scFv antibodies, a linear antibody, single domain antibody (sdAb), e.g., either a variable light (VL) chain or a variable heavy (VH) chain, a camelid VHH domain, and multispecific antibodies formed from antibody fragments. Antibody molecules can be polyclonal or monoclonal. The term “monoclonal” as applied to antibody molecules herein, refers to a population of antibody molecules that contain only one species of an antigen binding site capable of immunoreacting with a particular epitope.

A “marker”, or “tag” as used herein, refers to a molecule that can be used for identification, detection, purification, or isolation. In an embodiment, the marker comprises a small molecule, a peptide, a polypeptide, or a labeled amino acid or nucleotide. In an embodiment, the marker generates a signal for detection, e.g., a radioactive signal, a chemiluminescent signal, a fluorescent signal, or a chromogenic signal. For example, the marker is a dye, a fluorophore, a reporter enzyme (e.g., a photoprotein, luciferase), a fluorescent peptide, or a radionuclide. The generated signal can be detected by a variety of assays known in the art, such as fluorescence microscopy, fluorescence-activated cell sorting, gel electrophoresis, and spectrophotometry.

The terms “enhance” and “increase” refer to an increase in the specified parameter of at least about 1.25-fold, 1.5-fold, 2-fold, 3-fold, 4-fold, 5-fold, 6-fold, 8-fold, 10-fold, twelve-fold, or even fifteen-fold.

The terms “inhibit” and “reduce” or grammatical variations thereof as used herein refer to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible entity or activity (at most, an insignificant amount, e.g., less than about 10% or even 5%). As used herein, “complex” means an assemblage or aggregate of molecules in direct or indirect contact with one another. As used herein, “contact,” or more particularly, “contacting” with reference to an individual or complex of molecules, means two or more molecules are close enough that weak noncovalent chemical interactions, such as Van der Waal forces, hydrogen bonding, ionic and hydrophobic interactions, and the like, dominate the interaction of the molecules Generally, a complex of molecules is stable in that under assay conditions the complex is thermodynamically more favorable than a non-aggregated state of its component molecules.

As used herein, a “moiety” or “motif” comprises an amino acid, peptide, polypeptide, sugar, nucleic acid or other biological molecule having a structure that can be recognized and bind with another molecule.

A “target” or “target peptide” as the term is used herein, refers to a molecule that has affinity for a target recognition motif, or a target peptide that has affinity for a cleavage enzyme, such as Cap3.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Cap2-CD-NTase Structure

To characterize the basis for the Cap2-CD-NTase interaction, Applicants purified a stoichiometric Cap2-CD-NTase complex from a related CBASS found in Enterobacter cloacae and determined a 2.7A resolution structure by cryo-electron microscopy (cryo-EM) (FIG. 2a,b, Extended Data FIG. 1i-q, Extended Data Table 1 and Supplementary FIG. 2). The structure reveals a 2:2 complex with a homodimer of Cap2 bound to two CD-NTase monomers (FIG. 2c). Cap2 adopts a modular architecture with three domains: an N-terminal E2-like domain, a central linker domain, and a C-terminal E1-like domain (FIG. 2a). In eukaryotes, E1 and E2 domains catalyze the linkage of ubiquitin and related β-grasp fold proteins (collectively termed ubiquitin-like proteins or Ubls) to amine groups, typically lysine residues on target proteins. This process begins when an E1 conjugates adenosine monophosphate (AMP) to the Ubl C terminus (adenylation), then forms a thioester bond between the Ubl C terminus and a catalytic cysteine in the E1. The Ubl is next shuttled to a cysteine residue on an E2, and is finally transferred to a target with the help of an E3 adapter protein.

In Cap2, the C-terminal adenylation+E1 domain forms a tight homodimer, similar to those observed in the bacterial E1 proteins MoeB and ThiF, which participate in sulfur metabolism. The central linker domain of each Cap2 protomer reaches over the E1 domain of its dimer mate, positioning the N-terminal E2 domain close to the active site of its dimer-related E1 domain (FIG. 2c,d). Each Cap2 protomer is bound to a monomer of the CD-NTase via a composite interface involving one E1 domain and the nearby, dimer-related E2 domain (FIG. 2c,d). Mutations in the observed interface reduced or eliminated Cap2 binding to the CD-NTase (Extended Data FIG. 2a,b). The bipartite Cap2-CD-NTase interaction appears to rigidify Cap2: a second structure from the same cryo-EM dataset shows that when the Cap2 dimer is bound to only one CD-NTase, the linker and E2 domains of the unbound Cap2 protomer become flexible and are not observed in the cryo-EM map (Extended Data FIG. 2c,d).

The overall structure of Cap2 shows high similarity to that of ATG7, a non-canonical E1 protein involved in autophagy in eukaryotes (Extended Data FIG. 3a-d). Similar to Cap2, ATG7 forms a homodimer through its C-terminal E1 domain. In both Cap2 and ATG7, the catalytic E1 cysteine is positioned on the ‘crossover loop’ that extends over the E1 adenylation active site, rather than within an α-helical insertion in this loop as in canonical E1 proteins (Extended Data FIG. 3a-c). Next, the ATG7 N-terminal domain shares a common fold with the central linker domain of Cap2 and drapes over the dimer-related E1 domain in a manner similar to the Cap2 linker domain. In both Cap2 and ATG7, this domain binds and positions the E2 domain for catalysis (Extended Data FIG. 3a-d). Finally, comparison of the Cap2 E2 domain and the two non-canonical E2 proteins involved in ATG7-mediated ubiquitination (ATG3 and ATG10) reveals several features, including an incomplete UBC fold and a characteristic hairpin loop bearing the catalytic cysteine, which distinguish this group from canonical E2 proteins (Extended Data FIG. 3a,e). The unambiguous similarity of Cap2 to ATG7, and the homology between the Cap2 E2 domain and ATG3 and ATG10, strongly suggest that these two systems share a common evolutionary origin distinct from canonical E1 and E2 machinery. Our structure suggests that Cap2 is an all-in-one transferase capable of protein ligation. Supporting this model, disruption of the Cap2 adenylation, E1 or E2 active sites eliminated the ability of V. cholerae CBASS to protect against phage infection (FIG. 2e and Extended Data FIG. 4a,c).

### Example 2: Cap2 Mediates CD-NTase Conjugation

All known E1 enzymes use Ubls as substrates for adenylation and eventual conjugation to targets, but type II CBASS does not encode a Ubl. Our structure of the Cap2-CD-NTase complex reveals that that the extreme C terminus of the CD-NTase (residues 375-381) is bound to the Cap2 adenylation active site and conjugated to an AMP molecule (FIGS. 2d and 3a, Extended Data FIG. 2e-k and Extended Data Table 2), suggesting that the CD-NTase, rather than a ubiquitin-related β-grasp protein, is the substrate of Cap2-mediated conjugation (here, ‘substrate’ denotes the equivalent to ubiquitin, whereas ‘target’ denotes the protein or molecule to which the substrate is conjugated). The reactive-intermediate state captured in our structure closely matches previous structures of activated Ubls bound to their cognate E1 proteins, consistent with the CD-NTase serving as the Cap2 substrate. Further, CD-NTase enzymes in type II CBASS possess extended, disordered C termini with a conserved C-terminal glycine or alanine residue, reminiscent of the C-terminal diglycine motif of ubiquitin (FIG. 3b, Extended Data FIG. 5a,b and Supplementary Table 2).

If the CD-NTase is the substrate of Cap2, mutating or deleting the CD-NTase C-terminus or mutating the Cap2 E1 active site should destabilize the Cap2-CD-NTase complex and disrupt CBASS signaling. Accordingly, when Applicants mutated the C-terminal glycine residue of the V. cholerae CD-NTase to glutamate (G436E), phage protection was lost (FIG. 3c and Extended Data FIG. 4b,d). Furthermore, the Cap2-CD-NTase interaction in bacterial cells was compromised upon mutation of the Cap2 adenylation active site, the E1 catalytic cysteine or the CD-NTase C terminus (FIG. 3d and Extended Data FIG. 4a,c). Of note, inactivation of the Cap2 E2 catalytic cysteine did not disrupt the interaction with the CD-NTase FIG. 3d). Applicants anticipate that the E2 mutation preserves adenylation and conjugation of the CD-NTase to the E1 catalytic cysteine, trapping an intermediate state. In parallel, Applicants established an in-cell Cap2 activity assay and found that, Cap2-mediated CD-NTase conjugation indeed depends on the Cap2 adenylation active site, E1 and E2 catalytic cysteines, and the CD-NTase C terminus (FIG. 3e and Extended Data FIG. 4e-1).

Applicants hypothesized that the CD-NTase C-terminus is transferred by Cap2 to target molecules, in a similar manner to how ubiquitin is transferred to target molecules and tested this by probing for high molecular weight CD-NTase-target conjugates in vivo. Western blots showed the presence of several high-molecular-weight CD-NTase species in bacteria expressing the capV-cd-ntase-cap2 operon. These species disappeared when any of the catalytic functions of Cap2 were disrupted (FIG. 3f). In sum, these data demonstrate that Cap2 acts as a transferase, catalyzing the conjugation of the CD-NTase to an unidentified target.

### Example 3: Cap2 Primes cGAMP Synthesis

To understand the functional consequences of Cap2-mediated CD-NTase conjugation, Applicants immunoprecipitated the CD-NTase from bacteria expressing the capV-cd-ntase-cap2 operon and measured cGAMP synthesis by the purified protein. Applicants found that CD-Ntase purified from cells expressing wild-type Cap2 was significantly more active than CD-NTase from cells expressing Cap2 with E1 or E2 active-site mutations (FIG. 3g and Extended Data FIG. 5c-f). These data demonstrate that cGAMP synthesis is enhanced by Cap2 activity. The activity of purified CD-NTase was not altered by phage infection (FIG. 3h). These findings are consistent with the observation that the Cap2-CD-Ntase complex is unaffected by phage infection (FIG. 1d,e). These data suggest that Cap2 covalently modifies the CD-NTase prior to phage infection to prime or license CBASS signaling, and that a further phage-mediated trigger is needed in cells to fully activate second messenger synthesis.

Applicants next used mass spectrometry to identify the target or targets to which Cap2 conjugates the CD-NTase. Applicants immunoprecipitated CD-NTase from bacteria expressing the capV-cd-ntase-cap2 operon with either wild-type or E1-mutant cap2 alleles and quantified differentially enriched peptides (Extended Data FIG. 8a-f and Supplementary Table 8). Applicants identified 20 potential targets that were significantly enriched in samples expressing wild-type versus E1-mutant Cap2, with a strong bias toward proteins involved in metabolism (Extended Data FIG. 8d and Supplementary Table 8). These data are consistent with a contemporary analysis of Cap2-mediated CD-NTase conjugation in an E. coli CBASS. To determine whether conjugation of the CD-Ntase C terminus to an arbitrary target protein is sufficient to increase CD-NTase activity, Applicants measured cGAMP synthesis by CD-NTases fused to a C-terminal VSV-G tag, to GFP, or linked to the E1 active site of Cap2 (in a Cap2 E2 mutant). cGAMP synthesis by these proteins was equivalent to that by unconjugated CD-NTase (FIG. 3d,g and Extended Data FIGS. 5c-h and 7a,b), suggesting that CD-NTase activation depends on conjugation to a particular target protein or molecule. Future studies are required to establish the nature of the C-terminal modification and how it increases CD-NTase activity.

### Example 4: Cap3 Antagonizes Cap2

CBASS systems that encode Cap2 invariably also encode Cap3, which is homologous to eukaryotic JAB/JAMM-family ubiquitin proteases (FIG. 1a and Extended Data FIG. 6h). Applicants hypothesized that this protein balances CBASS activation by proteolytically cleaving CD-NTase-target conjugates. Although deletion of cap3 had no effect on CBASS-mediated phage resistance (FIG. 1b and Extended Data FIG. 1b), overexpression of Cap3 during infection strongly antagonized phage resistance (FIG. 4a and Extended Data FIG. 6c). To directly measure Cap3 activity, Applicants incubated V. cholerae and E. cloacae Cap3 with model substrates comprising their cognate CD-NTases fused at their C terminus to GFP. Both Cap3 proteins precisely cleaved the CD-NTase-GFP fusions at the CD-NTase C terminus, and this activity depended on conserved catalytic residues in the Cap3 active site and on Zn2+, which is required for catalysis by JAB/JAMM family proteases (FIG. 4b,c Extended Data FIG. 7 and Supplementary Table 3). V cholerae and E. cloacae Cap3 were unable to cleave substrates with mutations in the C-terminal region of their cognate CD-NTases (Extended Data FIG. 7).

To further define the specificity of Cap3 in vivo, Applicants overexpressed Cap3 alleles from four unrelated CBASS operons in combination with each of their cognate and non-cognate CBASS. Each Cap3 protein specifically antagonized phage protection by its cognate CBASS operon (FIG. 4d and Extended Data Fig. ad-g), demonstrating that Cap3 is exquisitely specific for its cognate CD-NTase.

Finally, Applicants tested the ability of Cap3 to antagonize CD-NTase-target conjugates in cells. Overexpression of wild-type Cap3, but not a catalytically dead mutant, eliminated the formation of Cap2-dependent high molecular weight CD-NTase species (FIG. 4e). Accordingly, Cap3 also antagonized cGAMP synthesis by CD-NTase immunoprecipitated from bacteria expressing Cap2 (FIG. 4f and Extended Data FIG. 5c-f). These data strongly support a model in which CD-NTase activation is primed by Cap2-mediated conjugation and antagonized by Cap3-mediated cleavage of CD-NTase-target conjugates (FIG. 5a).

### Example 5: Bacterial E1, E2 and JABs are Widespread

Antiphage systems continuously recombine and reassort into novel formulations that help bacteria gain an advantage in their conflict with phages. Applicants hypothesized that Cap2 and Cap3 homologues might be found in other antiphage systems and searched for these genes in the CBASS-related pyrimidine cyclase system for antiphage resistance (Pycsar). Pycsar encodes the phage-responsive cyclase protein PycC that generates a cyclic mononucleotide second messenger to activate an effector protein (FIG. 5b), analogous to type I CBASS.

Applicants identified a group of Pycsar, which Applicants term type II Pycsar, that encode an E2-E1 fusion protein homologous to Cap2 (Pap2: PycC associated protein 2) and a protein homologous to Cap3 (Pap3) (FIG. 5c). Supporting a model in which PycC is a substrate of Pap2 and Pap3, PycC proteins from type II, but not previously identified (type I) Pycsar38, have a highly conserved C-terminal extension (FIG. 5b,c) that structure predictions suggest binds to the Pap3 active site in a manner similar to CD-NTase-Cap3 binding (Extended Data FIG. 8g).

Previous bioinformatic studies have identified at least five distinct families of bacterial operons encoding predicted E1, E2 and JAB domain proteins, one of which is now understood to be type II CBASS. Applicants found that one such family encodes predicted metallo-p-lactamase (MBL) alongside a Cap2-like E2-E1 protein fused to a C-terminal JAB domain (FIG. 5d and Extended Data FIG. 8h,k). In this family, the C-terminal region of MBL possesses a conserved glycine residue four to six amino acids upstream of the C terminus (Extended Data FIG. 8j), which suggests that the JAB domain processes a pro-form of the MBL prior to E2-E1-mediated conjugation; this idea is supported by structural predictions (Extended Data FIG. 8i). Applicants tested the idea that JAB domains can process pro-substrates by appending a VSV-G tag to the C terminus of CD-NTase. In our type II CBASS, cap3 was required for phage resistance only when the C-terminal VSV-G was present, probably to remove the VSV-G tag in vivo and expose the native CD-Ntase C terminus for Cap2-mediated conjugation (Extended Data FIG. 6a,b). These findings may also explain why a gene encoding a JAB domain from the recently described bacterial ISG15-like system is essential for phage resistance.

Inspection of other operon families encoding E1, E2 and JAB domains shows that these operons also encode β-grasp fold Ubl proteins homologous to ubiquitin (FIG. 5d and Extended Data FIG. 8k). Overall, the abundance of bacterial operons encoding predicted E1, E2 and JAB-like proteins along with Ubls and other substrate proteins suggests that ubiquitin-like protein conjugation is widespread in bacteria.

### Example 6: CBASS Regulation

Here, Applicants have shown that the CBASS protein Cap2 is structurally homologous to ubiquitin transferases and conjugates a bacterial CD-Ntase to an unidentified target molecule. The covalent CD-NTase adduct is primed for cGAMP synthesis and is essential for phage defense. How CD-NTase-target conjugation primes the CD-NTase for activation is unknown, but our finding that priming is independent of phage infection suggests that additional phage cues are required for full CD-NTase activation in vivo. CD-NTase priming can be reversed by Cap3, a sequence-specific protease (FIG. 5a). In mammals, ubiquitination is also required to prime the innate immune receptor RIG-I, which enhances signaling. Together with our findings, this suggests that E1- and E2-domain-mediated protein conjugation may represent a conserved mechanism of immune regulation across kingdoms.

Although other bacterial proteins that catalyze ubiquitin conjugation have been identified, our findings reveal Cap2 as an all-in-one ATP-dependent ubiquitin transferase-like protein that uniquely combines adenylation, E1 and E2 active sites into a single polypeptide. Given the lack of an E3 protein in CBASS operons and the apparent low specificity of Cap2-mediated conjugation, Applicants hypothesize that target recognition is mediated directly by Cap2. The high similarity of Cap2 to non-canonical E1 and E2 transferases from eukaryotes (ATG7, ATG3 and ATG10) suggests that these systems share a common evolutionary origin. Thus, although ancestors of canonical ubiquitin signaling are found throughout eukaryotes and in some archaea, E1 and E2 transferases may have evolved first in bacteria, in line with previous bioinformatic observations. To our knowledge, CD-NTases are the only known substrates of ubiquitin transferase-like systems that do not share the β-grasp fold of Ubls. The all-in-one nature of Cap2, and its unique mode of substrate recognition, may enable engineering of this system to mediate customizable post-translational modifications.

In contrast to type I CBASS-which encodes only a CD-NTase and effector-type II CBASS encodes Cap2 and Cap3, which may increase CBASS sensitivity or license the CD-NTase to control inappropriate or spurious activation. CBASS operons with cap2 always encode cap3, suggesting that although cap3 is dispensable for phage resistance, it nonetheless provides a fitness advantage. The Cap2-Cap3 signaling scheme is reminiscent of type III CBASS, which encode HORMA-like proteins (Cap7-Cap8)—required for CD-NTase activation—and a TRIP13-like protein (Cap6) that disassembles activated CD-NTase-HORMA and primes HORMA proteins for peptide binding and CD-NTase activation. The apparent dual roles of Cap6 in type III CBASS suggests that Cap3 may also have two roles: first, to limit spurious CD-NTase priming and activation, and second, to disassemble non-specific CD-NTase conjugates to recycle CD-NTase that can be specifically primed for activation. Together, our findings show that diverse CBASS systems use multifaceted positive and negative regulators to finely control the activation of CD-NTase/DncV-like enzymes and mediate broad antiphage immunity.

### Example 7: Evolutionary Relationship Between Cap2 and the Autophagy-Related Non-Canonical E1 (ATG7) and E2 (ATG3 and ATG10) Proteins

The evolutionary origin of eukaryotic ubiquitin signaling pathways has long been a topic of intense interest. All E1 proteins likely evolved from bacterial enzymes related to the homodimeric E1s MoeB and ThiF, whseveralich adenylate the C-terminus of the b-grasp fold, ubiquitinlike proteins (Ubls) MoaD and ThiS, respectively, for metabolic cofactor synthesis. Also in bacteria, several families of operons encoding different combinations of Ubl, E1, E2, and JAB peptidase have been identified but not functionally characterized, leaving open the question of whether these systems mediate protein transfer. Five distinct bacterial operon types were originally described (termed families 6A-6E), of which Cap2 and Cap3-containing CBASS systems are one (family 6B). More recently, a sixth family (bilABCD) was described with similar domains. Recent data have shown that many archaea possess E1, E2, and RING-family E3 ligases that together conjugate a Ubl to target proteins, providing strong evidence that eukaryotic ubiquitin signaling evolved from these archaeal systems. The evolutionary origin of the non-canonical eukaryotic E1 and E2 proteins ATG7, ATG3, and ATG10, which conjugate the Ubls ATG12 and ATG8 to a target protein (ATG5) and a phospholipid (phosphatidylethanolamine), respectively, are less well defined.

Several lines of evidence support an evolutionary relationship between Cap2 and the autophagy-related non-canonical E1 (ATG7) and E2 (ATG3 and ATG10) proteins. First, unlike most eukaryotic E1 s that adopt a pseudo-homodimeric architecture with a single active adenylation site, the C-terminal E1 domain of Cap2 forms a homodimer with two active adenylation sites, like MoeB, Thi, and ATG7 (Extended Data FIG. 3b,c). The ATG7 E1 domain is also more closely related to bacterial E1 s in the 6A and 6B/Cap2 families than it is to other eukaryotic E1 proteins. Second, while the catalytic cysteine residues of most eukaryotic E1 proteins are located on an a-helical domain inserted in the crossover loop of their adenylation domain, the catalytic cysteines of both Cap2 and ATG7 are located on these proteins' crossover loop, with no a-helical domain insertion (Extended Data FIG. 3c). Thus, Cap2 and ATG7 share a common structural mechanism of substrate adenylation and transfer to the E1 catalytic cysteine.

In most eukaryotic E1 proteins, a ubiquitin fold domain (UFD) positioned C-terminal to the adenylation domain recruits and positions E2 proteins for substrate transfer. In ATG7, however, the protein's structurally distinctive N-terminal domain recruits and positions two different non-canonical E2 proteins, ATG3 and ATG10, for catalysis. The central linker domain of Cap2 shares a common fold with the ATG7 N-terminal domain (Extended Data FIG. 3d) and is positioned similarly in the dimeric assembly, with each linker domain draping over its dimeric partner's adenylation active site (Extended Data FIG. 3c). While the ATG7 N-terminal domain recruits separate E2 proteins, the Cap2 linker domain positions its own N-terminal E2 domain close to the adenylation site and E1 catalytic cysteine. Thus, the role of this domain in positioning E2 for catalysis is shared between Cap2 and ATG7.

In addition to similarities between Cap2 and ATG7, the Cap2 N-terminal E2 domain is structurally related to the non-canonical E2 proteins ATG3 and ATG10 that play a role in autophagy. Canonical E2 proteins contain a UBC fold, which comprises a four-stranded b-sheet surrounded by four a-helices, with the catalytic cysteine located on a loop between b4 and a2. Like ATG3 and ATG10, the Cap2 E2 domain lacks a-helices 3 and 4 (Extended Data FIG. 3e), and also lacks other conserved features of canonical E2 proteins, including an HPN motif ˜8 amino acids N-terminal to the catalytic cysteine, a conserved tryptophan residue C-terminal to the catalytic cysteine, and a proline-rich motif between b-strands 3 and 4. Further, the catalytic cysteine of the Cap2 E2 domain (C109) is positioned on a long hairpin loop that is structurally analogous to the extended b5-b6 hairpin found in both ATG3 and ATG10 (Extended Data FIG. 3e).

Cap2 possesses strong structural similarity to the autophagy E1 ATG7 and to its cognate E2s ATG3 and ATG10. Thus, despite important differences between Cap2 and the autophagy E1/E2 proteins, including distinct substrates (CD-NTase versus Ubl proteins) and protein architecture (a single polypeptide versus separate E1 and E2 proteins), Applicants conclude that these pathways share a common bacterial ancestor distinct from the archaeal ancestors of other eukaryotic Ubl transfer pathways.

### Example 8: Materials and Methods

### Bacterial Strains and Growth Conditions:

E. coli strains used in this study are listed in Supplementary Table 4. E. coli were cultured in LB medium (1% tryptone, 0.5% yeast extract, 0.5% NaCl) shaking at 37° C., 220 rpm unless otherwise noted. For phage experiments and other noted assays, bacteria were grown in MMCG minimal medium containing M9 salts, magnesium, calcium, and glucose (47.8 mM Na2HPO4, 22 mM KH2PO4, 18.7 mM NH4Cl, 8.6 mM NaCl,

22.2 mM glucose, 2 mM MgSO4, 100 μM CaCl₂), 3 μM thiamine). Where applicable, media were supplemented with carbenicillin (100 μg ml-1) or chloramphenicol (20 μg ml-1), to ensure plasmid maintenance. When a strain with two plasmids was cultivated in MMCG medium, bacteria were cultured with 20 μg ml-1 carbenicillin and 4 μg ml-1 chloramphenicol. Applicants defined an overnight culture as 16-20 h post-inoculation from a single colony or glycerol stock. All strains were stored in LB plus 30% glycerol at −70° C. E. coli OmniPir47 was used for plasmid construction and propagation and E. coli MG1655 (CGSC6300) was employed for all experimental data.

### Plasmid Construction:

Plasmids used in the study are listed in Supplementary Table 4. All experiments were performed with either the CBASS system from V. cholerae C6706 (NCBI RefSeq NZ_CP064350.1) or E. cloacae (NCBI Ref-Seq NZ_KI973084.1; see protein accession numbers in Supplementary Table 5) with the exception of the Cap3 overexpression experiments presented in FIG. 4d and Extended Data FIG. 6d-g which also used the CBASS operons associated with CD-NTase 38, 42 and 127 (NCBIRefSeq WP_032676400, WP_000992191.1, and WP_052435251.1). For phage infections, the entire operon plus surrounding sequences were cloned into the XhoI and NotI sites of the vector pLOCO247. For in vivo cap3 expression, genes were cloned into the BamHI and NotI sites of the vector pTACxc. pTACxc (full sequence available in Supplementary Table 4) was constructed by combining the ColEl origin of replication from pBAD2448, chloramphenicol resistance from pBAD18cm48, the RP4 oriT, laclq from OmniPir E. coli, a Ptac promoter49 and superfolder GFP (sfGFP). For immunoprecipitation assays, a C-terminal 3×Flag tag was added to V. cholerae cap2 and an N-terminal VSV-G tag was added to V. cholerae CD-NTase. A C-terminal VSV-G tag was added to V. cholerae CD-NTase to test target specificity. For biochemical analysis, individual proteins were cloned into vector 2-BT (Addgene #29666; N-terminal His6-TEV cleavage site fusion), H6-msfGFP (Addgene #29725; N-terminal His6-TEV cleavage site fusion and C-terminal msfGFP fusion), or 2-AT (Addgene #29665; untagged).

For E. cloacae Cap3, sequence alignments revealed that the first 16 codons of the annotated gene are unlikely to be translated in vivo; a truncated construct comprising residues 17-180 of the annotated gene expressed at higher levels and was more soluble upon purification (for mutations, residue numbering follows the annotated gene). For E. cloacae Cap2-CD-NTase complex used for cryo-EM, the two genes were amplified by PCR from vector 2-AT and combined to generate a polycistronic transcript, then cloned into vector 2-BT resulting in an N-terminal His6-tag on CD-NTase and no tag on Cap2, and both catalytic cysteine residues in Cap2 (C109 and C548) were mutated to alanine.

For the E. cloacae Cap2-CD-NTase complex used in the Cap2 activity assay, the two genes were cloned as above into vector 2-BT to generate a polycistronic transcript with an N-terminal His6-tag on Cap2 and no tag on CD-NTase. For E. cloacae Cap2-CD-NTase complex with haemagglutinin (HA)-tagged CD-NTase, the two genes were cloned as above into vector 2-AT to generate a polycistronic transcript with an N-terminal HA tag (MYPYDVPDYAGSG) fused to residue 2 of CD-NTase. DNA sequences were cloned into destination vectors using 18-25 bp overhangs and Gibson Assembly. Point-mutations and epitope tags were cloned by mutagenic PCR and isothermal assembly. Clones were transformed either into a modified strain of OmniMax E. coli (Invitrogen) by electroporation, or into NovaBlue E. coli (Novagen) by heat-shock and plated on LB with the appropriate selection. Positive clones were verified by Sanger Sequencing (Genewiz). Prior to use in downstream phage or immunoprecipitation experiments, sequence verified plasmids were transformed into MG1655 via heat shock and plated on LB with the appropriate selection.

### Phage Amplification and Storage:

Phages used in the study are listed in Supplementary Table 6. Phage lysates were generated from E. coli MG1655 using a modified double agar overlay plate amplification (T2) or liquid amplification (T4, T5 and T6). For plate amplification, stationary phase MG1655 was infected with 10,000 plaque-forming units (PFU) of phage in LB+0.35% agar, 10 mM MgCl₂, 10 mM CaCl₂), and 100 μM MnCl2. Plates were incubated overnight (16-20 h) at 37° C. and the following day phages were collected by adding 5 ml of SM buffer (100 mM NaCl, 8 mM MgSO4, 50 mM Tris-HCl pH 7.5, 0.01% gelatin) directly to the plate, incubating for 1 h at room temperature, then collecting and filtering the resulting liquid through a 0.2 m Nanosep filter. For liquid amplification, early logarithmic phase MG1655 was infected at an MOI of 0.1 in 25 ml LB broth plus 10 mM MgCl₂, 10 mM CaCl₂), and 100 μM MnCl₂ at 37° C. with 220 rpm shaking for 2-6 h until the culture became clear. Supernatants were then collected via centrifugation and filtration with a 0.2 m Nanosep filter. Lysate titres were determined by spotting a serial dilution of the phage onto 0.35% LB agar plus 10 mM MgCl₂, 10 mM CaCl₂), and 100 μM MnCl₂ containing stationary phase MG1655. Plates were incubated overnight at 37° C. and the resulting titre in PFU ml-1 was calculated. Phage stocks were stored at 4° C. in either SM buffer or LB broth.

### Efficiency of Plating and Phage Infection Assays:

Phage protection assays were performed using a modified double agar overlay technique. Bacteria were cultivated overnight in MMCG medium, and the following day were diluted 1:10 into fresh medium and grown until mid-logarithmic phase. Four-hundred microlitres of MG1655 containing the indicated vector(s) was inoculated into 3.5 ml 0.35% MMCG agar, mixed, and poured on top of a conventional MMCG 1.6% agar plate. For the Cap3 overexpression experiments, 0, 50 or 500 μM IPTG was added to both the bacterial culture and the top agar. The plate was allowed to cool and dry for ˜10 min after which 2 μl of phage serial dilution was spotted onto the soft agar overlay. After phage spots dried, plates were incubated at 37° C. overnight. Plates were imaged ˜24 h after infection and PFU were enumerated. The resulting efficiency of plating for each phage was measured by quantifying titre in PFU ml-1 for each phage lysate tested. PFU were enumerated for phage dilution spots with 1-30 PFU, then the dilution was used to scale to PFU ml-1 appropriately. When individual plaques could not be counted and instead a hazy zone of clearance was observed, the lowest phage concentration at which Applicants could detect this clearance was counted as ten plaques. When no clearance was observed, 0.9 plaques at the least dilute spot were used as the limit of detection for that assay (see Extended Data FIG. 1a for an example). The data are presented as fold protection compared to a control strain expressing GFP, which is simply the inverse of the efficiency of plating. Data are shown as the mean±s.e.m. of three biological replicates. Statistical significance, as determined by an unpaired two-sided Student's t-test, is shown when applicable. For large and obviously significant differences between data, such as greater than 100-fold, statistics are not indicated, for clarity. All the raw data from these experiments along with the relevant P-values are found in Supplementary Data 1.

### Immunoprecipitation Assays:

MG1655 E. coli expressing the indicated vectors were grown to mid-logarithmic phase in MMCG. Where listed, cells were infected with the indicated phage for 30 min (or as noted) at a MOI of 2. Cultures were then centrifuged, and the resulting pellet was resuspended in lysis buffer (400 mM NaCl, 20 mM Tris-HCl pH 7.5, 2% glycerol, 1% Triton X-100 and 1 mM 2-mercaptoethanol). Cells were disrupted by sonication followed by centrifugation at 4° C. to remove cellular debris. Soluble lysates were then mixed with the epitope tag purification resin, as described below, overnight at 4° C. with end-over-end rotation. The following day, samples were washed 5 times in 1-5 ml lysis buffer and beads were processed for downstream application. For CD-Ntase immunoprecipitations, lysates were incubated with either protein A magnetic beads (Pierce) containing 10 μg ml-1 CD-NTase antibody or, when CD-NTase had a VSV-G tag, with agarose beads conjugated to an anti-VSV-G antibody (Sigma). Cap2-3×Flag was immunoprecipitated using magnetic beads covalently linked to the anti-Flag M2 antibody (Sigma).

### Western Blots:

Rabbit CD-NTase polyclonal antibody was generated by a commercial vendor (Genescript) using a purified, untagged CD-NTase antigen. Polyclonal CD-NTase antibodies were further purified by antigen affinity (GenScript). Serum was used at 1:30,000 for CD-NTase immunoblot detection. Flag antibody (Sigma) was used at 1:10,000 to detect Cap2-3×Flag, anti-VSV-G (Rockland) was used at 1:7,500 to detect VSV-G tagged CD-NTase, anti-RNAP (Biolegend) was used at 1:5,000 for use as a loading control, and anti-HA (clone 3F10, Sigma-Aldrich) was used at 1:30,000 to detect HA-tagged proteins. For whole-cell lysate analysis, 5 ml of MG1655 carrying the indicated plasmid were grown to mid-logarithmic phase. Cell densities were then normalized and 5×109 CFU were collected, centrifuged and resuspended in 50 μl of 1×LDS buffer (106 mM Tris-HCl pH7.4, 141 mM Tris base, 2% w/v lithium dodecyl sulfate, 10% v/v glycerol, 0.51 mM EDTA, 0.05% Orange G). Samples were then incubated at 95° C. for 10 min followed by a 5-min centrifugation at 20,000 g to remove debris. For immunoprecipitation samples, affinity purification beads were resuspended in 40 μl lysis buffer plus 40 μl 2×LDS buffer. Samples were then incubated at 95° C. followed by a 5-min centrifugation at 20,000 g.

Samples in LDS were loaded at equal volumes to resolve by SDS-PAGE, then transferred to PVDF membranes charged in methanol. Membranes were blocked in Licor Intercept Buffer for 30 min at 24° C., followed by incubation with primary antibodies diluted in Intercept buffer overnight at 4° C. Blots were then incubated with the appropriate combination of Licor infrared (800CW/680RD) anti-rabbit or anti-mouse secondary antibodies at 1:30,000 dilution in TBS-T (0.1% Triton-X) for 45 min at 24° C. and visualized using a Licor Odyssey CLx. For anti-HA immunoblots, horseradish peroxidase-linked goat anti-rat antibody (Pierce 31470) was used at 1:30,000 and detected with a HRP Substrate kit (Bio-Rad) and Bio-Rad ChemiDoc imager. Representative images were assembled using Adobe Illustrator CC 2022.

### Mass Spectrometry Analysis:

Following enrichment by immunoprecipitation as described above, samples were subjected to on-bead trypsin digest followed by analysis on a Thermo Obitrap Q-Exactive HF-X using nanoLC-MS/MS. Peptideswere mapped to the proteome of E. coli MG1655 (uniprot.org/proteomes/UP000030788), the proteins comprising the CBASS operon from V. cholerae (CapV, CD-NTase, Cap2 and Cap3) and the proteome of the phage T2 (https://www.uniprot.org/proteomes/UP000503557), which was used to infect the samples. Peptides were considered significantly enriched when their label-free quantification (LFQ) score was >108 and they were more than fourfold enriched over the Cap2(C522A(E1)) samples.

### CD-NTase Enzyme Assay

A total of 6.25×109 CFU of MG1655 cells expressing the indicated plasmids were processed for immunoprecipitation enrichments as described above using 20 μl bead volume. Of note, the experiments described in FIG. 3g,h used a vector in which cap3 had been deleted. Anti-VSV-G agarose beads were further washed three times in 1 ml reaction buffer (50 mM Tris-HCl pH 7.5, 50 mM KCl, 5 mM MgCl2). Samples were then resuspended in 120 μl reaction buffer, split into three technical replicates and incubated with 500 μM ATP and 500 μM GTP overnight at 37° C. followed by 10 min at 95° C. to inactivate the CD-NTase. cGAMP levels were then quantified with the Arbor Assay 3′,3′-cGAMP ELISA per the manufacturer's specifications. When necessary, samples were diluted 1:5 and 1:25 in the provided assay buffer to ensure measurements were within the dynamic range. cGAMP levels were calculated using the provided standard measured in triplicate. Data shown are a representative graph from one of three independent experiments depicting the mean±s.d. of three technical replicates. To allow for accurate comparison of all the ELISA data see Extended Data FIG. 6c,d. For in vitro second messenger synthesis assays, 1-ml reactions with 1 μM CD-NTase or CD-NTase-GFP fusion proteins were mixed with reaction buffer containing 10 mM Tris-HCl pH 8.5, 12.5 mM NaCl, 20 mM MgCl2, 1 mM DTT, 0.25 mM ATP and 0.25 mM GTP, then incubated at 37° C. for 16 h. Ten units of calf intestinal phosphatase (Quick CIP, New England Biolabs) were added and further incubated at 37° C. for 2 h. Reactions were stopped by heating at 65° C. for 20 min, then centrifuged at 15,000 rpm for 10 min to remove precipitated protein. Reaction products were separated by anion-exchange chromatography (1 ml Hitrap Q HP, Cytiva) using a gradient from 0.5 to 2 M ammonium acetate, on an Akta PURE 25M FPLC (Cytiva). Products were quantified by peak-area integration from the A254 absorbance profile using Unicorn v. 7.3 (Cytiva).

### Protein Alignments:

Cap2 and Cap3 protein alignments were generated with the MUSCLE algorithm51 within Geneious software, then adjusted by hand based on structure superpositions performed using the PDBeFold server (ebi.ac.uk/pdbe/). Sequence logos were generated using WebLogo (weblogo.berkeley.edu/logo.cgi).

### Protein Expression and Purification:

Protein expression vectors used in the study are listed in Supplementary Table 4. For protein purification, expression vectors were transformed into E. coli Rosetta2 pLysS (EMD Millipore) or LOBSTR (Kerafast), grown at 37° C. in 2× YT media to anA600 of 0.6, then protein expression was induced by the addition of 0.25 mM IPTG. Cultures were shifted to 20° C. for 16 h, then cells were collected by centrifugation. Cells were resuspended in binding buffer (25 mM Tris-HCl pH 8.5, 5 mM imidazole, 300 mM NaCl, 5 mM MgCl2, 10% glycerol, and 5 mM 2-mercaptoethanol), lysed by sonication, and centrifuged (20,000 g for 30 min) to remove cell debris. Clarified lysate was passed over a Ni2+ affinity column (Ni-NTA Superflow, Qiagen) and eluted in a buffer with 250 mM imidazole. For cleavage of His6-tags, proteins were buffer-exchanged to binding buffer, then incubated 48 h at 4° C. with His6-tagged TEV protease52. Cleavage reactions were passed through a Ni2+ affinity column again to remove uncleaved protein, His6-tags, and TEV protease. Flow-through fractions were passed over a size-exclusion chromatography column (Superdex 200; Cytiva) in gel filtration buffer (25 mM Tris-HCl pH 8.5, 300 mM NaCl, 5 mM MgCl2, 10% glycerol, 1 mM DTT). Gel filtration buffer without glycerol was used for samples for cryoelectron microscopy. Purified proteins were concentrated and stored at −80° C. for analysis or 4° C. for crystallization.

### Cryo-EM:

For grid preparation, freshly purified E. cloacae Cap2-CD-NTase complex was collected from size-exclusion chromatography and diluted to 8 μM. Immediately prior to use, Quantifoil Cu 1.2/1.3 300 grids were glow-discharged for 10 s in a preset program using a Solarus II plasma cleaner (Gatan). Sample was applied to a grid as a 3.5 μl drop in the environmental chamber of a Vitrobot Mark IV (Thermo Fisher Scientific) Article held at 4° C. and 100% humidity. After a 1-min incubation, the grid was blotted with filter paper for 5 s prior to plunging into liquid ethane cooled by liquid nitrogen. Grids were mounted into standard AutoGrids (Thermo Fisher Scientific) for imaging. All samples were imaged using a Titan Krios G3 transmission electron microscope (Thermo Fisher Scientific) operated at 300 kV configured for fringe-free illumination and equipped with a K2 direct electron detector (Gatan) mounted post Quantum 968 LS imaging filter (Gatan). The microscope was operated in EFTEM mode with a slit-width of 20 eV and using a 100 m objective aperture. Automated data acquisition was performed using EPU (Thermo Fisher Scientific) and all images were collected using the K2 in counting mode. Ten-second movies were collected at a magnification of 165,000× and a pixel size of 0.84 A, with a total dose of 64.8 e-A-2 distributed uniformly over 40 frames. In total, 2,437 movies were acquired with a realized defocus range of −0.5 to −2.5 μm.

Cryo-EM data analysis was performed in cryoSPARC version 3.253 (Extended Data FIG. 1i-q and Extended Data Table 1). Movies were motion-corrected using patch motion correction (multi) and contrast transfer function (CTF)-estimated using patch CTF estimation (multi)54, and a 200-image subset was used for initial particle picking using the blob picker. Initial picks were subjected to 2D classification, and two classes were picked as templates for template-based particle picking of the entire 2,437-image dataset. Two-dimensional classification of the resulting ˜2.5 million particles revealed relatively few high-quality classes, likely a result of particle shape irregularity and high density on the grids. A subset of approximately 1 million particles was used for initial ab initio 3D reconstruction, then the entire ˜2.5 million particle dataset was used for heterogeneous refinement against these models, resulting in a 663,199-particle set that resulted in a ˜5.5 A resolution map with recognizable protein features. This particle set was subjected to a further round of ab initio reconstruction and heterogeneous refinement to separate 2:2 Cap2:CD-NTase complexes from 2:1 complexes. The two separate particle sets were cleaned with another round of 3Dclassifications, then re-extracted with a 440-pixel (370 A) box size and refined using the non-uniform refinement NEW job type incryoSPARCwith the following options enabled: maximize over-particle scale; optimize per-particle defocus; optimize per-group CTF params. For the 2:2 complex, C2 symmetry was applied during refinement. The resulting reconstructions showed resolution values of 2.74 A (2:2 complex) and 2.91 A (2:1 complex) using the 0.143 cut-off criterion of the Fourier shell correlations between masked independently refined half-maps. Resolution anisotropy for both reconstructions was assessed using the 3DFSC web server.

An initial model for E. cloacae Cap2 was generated by AlphaFold256. This model and the crystal structure of ATP-bound E. cloacae CD-Ntase (Protein Data Bank (PDB) ID 7LJL30) were manually docked into the final 2:2 complex cryo-EM map using UCSF Chimera57 and rebuilt in COOT58. For the E1 domain of Cap2 and for CD-NTase, high-resolution crystal structures were used to verify the accuracy of the resulting model. The final rebuilt model was real-space refined in phenix.refine59. This model was then docked into the 2:1 complex map, disordered regions were deleted, and the final model was real-space refined in phenix.refine59. Structure validation was performed with MoProbity60 and EMRinger61. Structures were visualized in ChimeraX57 and PyMOL (Schrodinger).

### Crystallography:

To determine a crystal structure of the Cap2 E1 domain bound to the CD-NTase C terminus in the apo state, Applicants cloned and purified a fusion construct with E. cloacae Cap2 residues 374-600 (C548A mutant) fused at its C terminus to a flexible linker and residues 370-381 of CD-Ntase (sequence: GSGKPAEPQKTGRFA). Purified protein was exchanged into a buffer containing 25 mM Tris-HCl pH 8.5, 200 mM NaCl, 5 mM MgCl2 and 1 mM TCEP, then concentrated to 30 mg ml-1. Small rod-shaped crystals grew in hanging drop format by mixing 1:1 of protein with well solution containing 0.1 M Tris-HCl pH 8.5, 0.8 M LiCl, and 25% PEG 3350. Crystals were transferred to a cryoprotectant containing an additional 10% glycerol, then flash-frozen in liquid nitrogen. Applicants collected a 1.77 A resolution diffraction dataset at NE-CAT beamline 24ID-C at the Advanced Photon Source at Argonne National Laboratory (Extended Data Table 2). Data were processed with the RAPD pipeline, which uses XDS62 for data indexing and reduction, AIMLESS63 for scaling, and TRUNCATE64 for conversion to structure factors. Applicants determined the structure by molecular replacement in PHASER65 using the refined Cap2 E1 domain structure from our cryo-EM model of Cap2-CD-NTase. The model was rebuilt in COOT58, followed by refinement in phenix.refine66 using positional, individual B-factor, and TLS refinement (statistics in Extended Data Table 2).

To determine a crystal structure of the Cap2 E1 domain bound to the CD-NTase C terminus in the AMP-bound reactive intermediate state, Applicants cloned and purified a fusion construct with E. cloacae Cap2 residues 363-600 (C548A mutant) fused at its C terminus to a flexible linker and residues 370-381 of CD-NTase (sequence: GSGKPAEPQKTGRFA). Purified protein was exchanged into crystallization buffer and concentrated to 30 mg ml-1. A final concentration of 2.5 mM ATP was added to the protein and incubated overnight at 4° C. Needle crystals grew in hanging drop format by mixing 1:1 of protein with well solution containing 0.1 M Tris-HCl pH 8.5, 0.2 M MgCl2, and 30% PEG 3350). Crystals were looped directly from the drop and flash-frozen in liquid nitrogen. A 2.11 A resolution diffraction dataset was collected at NE-CAT beamline 24ID-E at Advanced Photon Source at Argonne National Laboratory and processed as above.

### Cap2 and Cap3 Biochemical Assays:

For Cap2 activity assays, the indicated combinations of E. cloacae His6-Cap2 and untagged CD-NTase (wild-type or mutant) were co-expressed in Rosetta2 pLys E. coli cells, then purified as above using a Ni2+ affinity column. Samples were analysed by SDS-PAGE with Coomassie staining. For quantification, experiments were run in triplicate and Coomassie blue-stained bands quantified using Fiji software. For Cap3 activity assays, model substrates comprising E. cloacae or V. cholerae His6-CD-NTase (wild type or mutant) fused at their C terminus to GFP were cloned and purified as above. Model substrates (4.5 μg) were incubated with Cap3 (1.5 μg) in a reaction buffer with 20 mM HEPES pH 7.5, 100 mM NaCl, 20 mM MgCl₂, 20 μM ZnCl₂ and 1 mM DTT (20 μl total reaction volume). Reactions were incubated 30 min at 37° C., then analyzed by SDS-PAGE with Coomassie blue staining.

### Trypsin Mass Spectrometry:

For trypsin mass spectrometry of purified proteins (HA-CD-NTase and Cap2-GFP), in-gel digestion was performed according to a previously described method. In brief, proteins in diced gel bands were reduced with 100 μl of 10 mM DTT for 30 min at 37° C. and then alkylated with 6 μl of 0.5 M iodoacetamide in water for 20 min at room temperature in the dark. To digest proteins, 25-30 μl of 10 ng μl-1 trypsin (Promega, V511A) in 50 mM ammonium bicarbonate (pH 8) was added to cover the gel pieces and incubated on ice for 30 min until fully swollen. An additional 10-20 μl of ammonium bicarbonate buffer was added and the sample was incubated overnight at 37° C. The next day, trypsin digested peptides were extracted from the gel via multiple solvent extractions, dried under vacuum and then resuspended in 5 μl of 0.6% acetic acid. The digested peptides were analyzed by a Thermo Fisher Scientific Orbitrap Fusion LUMOS Tribrid mass spectrometer using a standard LC-MS/MS method.

### Mass Spectrometry Data Analysis was Performed Using the

Trans-Proteomic Pipeline (TPP, Seattle Proteome Center). In brief, mass spectrometry data were searched using the search engine COMET against a composite E. coli database that additionally contained protein sequences for E. cloacae CD-NTase and Cap2, plus common contaminants. Variable modifications include possible oxidation of methionine (15.9949 Da) and expected FA remnant of the CD-NTase C terminus (218.10552 Da); and a static modification of cysteine by IAA (57.021464 Da) was included. The COMET search results were further analyzed with PeptideProphet and ProteinProphet69. Peptides with a probability of >0.9 and mass accuracy of <10 ppm were subjected to further manual inspection of the MS/MS spectra to confirm major fragment ions are accounted for.

### Bioinformatic Analyses:

The CD-NTase alignments and tree in FIG. 3b and Extended Data FIG. 5 were adapted from previously published datasets2,6,35 (Supplementary Table 10). CD-NTase clades with more than around ˜75% of CD-NTases encoded adjacent to Cap2 and Cap3 homologues were deemed type II systems and highlighted. For each subset of CD-NTases indicated, C-terminal residues were extracted and aligned to create a sequence logo using WebLogo (weblogo.berkeley.edu/logo.cgi). Sequence logos in Extended Data FIG. 5a use data from ref 35, and sequence logos in FIG. 3b and Extended Data FIG. 5b use data from refs. 2,6. To identify Pycsar systems that contain E1, E2 and JAB domains Applicants initially searched the Integrated Microbial Genomes (IMG) database for homologues of the cyclase gene pycC from Pseudomonas aeruginosa (2736613764). All available hits for this gene were downloaded along with 10,000 bp upstream and downstream of the genes. The operon predictor Glimmer was used on each region of DNA and all identified genes were extracted and translated. Interpro was used to predict the protein domains found within each extracted protein sequence.

To identify proteins containing E1 and E2 domains, Applicants searched for the E1 protein domain ThiF. Applicants then confirmed that these sequences also encode for an E2- and JAB-domain-containing protein. All pycC genes that were associated with these domains were then extracted, translated and the last nine amino acids were aligned to generate a sequence logo. Applicants then broadened our search to include E1 and E2 domains that were previously reported. Applicants expanded upon their analysis and IMG was used to identify homologues of the genes encoding these proteins and a representative 500 genes and 10,000 bp upstream and downstream were extracted. Applicants again used Glimmer and Interpro to identify protein domains associated with E1 and E2 domains. From this analysis Applicants identified numerous operons that could be divided into four broad classes, those that contain an MBL domain, those with a CEHH domain, α-helical domain-containing operons, and finally those that contain a DUF6527 domain. Representatives of each operon architecture (FIG. 5d and Extended Data FIG. 8k) were identified. Data can be found in Supplementary Table 9. For structure and model predictions, Applicants used a local installation of ColabFold (github.com/YoshitakaMo/localcolabfold)70, which implements the AlphaFold56 and AlphaFold-Multimer71 algorithms.

### Statistics and Reproducibility:

All efficiency of plating phage assays were performed with n=3 independent biological replicates observed on different days. Data are presented as the mean±s.e.m. and a two-sided Student's t-test was used to calculate significance. NS, P>0.05; *P<0.05, **P<0.001. Actual P-values are listed in Supplementary Data 1. All western blots and Coomassie analysis presented are representative of n=3 independent biological replicates (see Supplementary FIG. 1 for uncut gel images). This includes FIGS. 1d,e, 3d,f and 4b,e and Extended Data FIGS. 1d,e,g,h, 2b, 4c-e,g,i,l, 6b, 7c,e,f and 8a,b. Mass spectrometry analysis was performed twice on two independent biological replicates for each experiment conducted.

### REFERENCES

1. Ni, G., Ma, Z. & Damania, B. cGAS and STING: At the intersection of DNA and RNA virus-sensing networks. PLOS Pathog. 14, e1007148 (2018).
2. Hopfner, K.-P. & Hornung, V. Molecular mechanisms and cellular functions of cGAS-STING signalling. Nat. Rev. Mol. Cell Biol. 21, 501-521 (2020).
3. Morehouse, B. R. et al. STING cyclic dinucleotide sensing originated in bacteria. Nature 586, 429-433 (2020).
4. Ye, Q. et al. HORMA Domain Proteins and a Trip13-like ATPase Regulate Bacterial cGAS-like Enzymes to Mediate Bacteriophage Immunity. Mol. Cell 77, 709-722.e7 (2020).
5. Cohen, D. et al. Cyclic GMP-AMP signalling protects bacteria against viral infection. Nature 574, 691-695 (2019).
6. Millman, A., Melamed, S., Amitai, G. & Sorek, R. Diversity and classification of cyclic-oligonucleotide-based anti-phage signalling systems. Nat. Microbiol. 5, 1608-1615 (2020).
7. Burroughs, A. M., Zhang, D., Schaffer, D. E., Iyer, L. M. & Aravind, L. Comparative genomic analyses reveal a vast, novel network of nucleotide-centric systems in biological conflicts, immunity and signaling. Nucleic Acids Res. 43, 10633-10654 (2015).
8. Whiteley, A. T. et al. Bacterial cGAS-like enzymes synthesize diverse nucleotide signals. Nature 567, 194-199 (2019).
9. Severin, G. B. et al. Direct activation of a phospholipase by cyclic GMP-AMP in E1 Tor Vibrio cholerae. Proc. Natl. Acad. Sci. 115, E6048-E6055 (2018).
10. Lowey, B. et al. CBASS Immunity Uses CARF-Related Effectors to Sense 3′-5′- and 2′-5′-Linked Cyclic Oligonucleotide Signals and Protect Bacteria from Phage Infection. Cell 182, 38-49.e17 (2020).
11. Lau, R. K. et al. Structure and Mechanism of a Cyclic Trinucleotide-Activated Bacterial Endonuclease Mediating Bacteriophage Immunity. Mol. Cell 77, 723-733.e6 (2020).
12. Duncan-Lowey, B., McNamara-Bordewick, N. K., Tal, N., Sorek, R. & Kranzusch, P. J. Effector-mediated membrane disruption controls cell death in CBASS antiphage defense. Mol. Cell 81, 5039-5051.e5 (2021).
13. Davies, B. W., Bogard, R. W., Young, T. S. & Mekalanos, J. J. Coordinated Regulation of Accessory Genetic Elements Produces Cyclic Di-Nucleotides for V. cholerae Virulence. Cell 149, 358-370 (2012).
14. Dziejman, M. et al. Comparative genomic analysis of Vibrio cholerae: genes that correlate with cholera endemic and pandemic disease. Proc. Natl. Acad. Sci. U.S.A 99, 1556-1561 (2002).
15. Nandi, D., Tahiliani, P., Kumar, A. & Chandu, D. The ubiquitin-proteasome system. J. Biosci. 31, 137-155 (2006).
16. Cappadocia, L. & Lima, C. D. Ubiquitin-like Protein Conjugation: Structures, Chemistry, and Mechanism. Chem. Rev. 118, 889-918 (2018).
17. Pickart, C. M. Mechanisms Underlying Ubiquitination. Annu. Rev. Biochem. 70, 503-533 (2001).
18. Lake, M. W., Wuebbens, M. M., Rajagopalan, K. V. & Schindelin, H. Mechanism of ubiquitin activation revealed by the structure of a bacterial MoeB-MoaD complex. Nature 414, 325-329 (2001).
19. Xu, X., Wang, T., Niu, Y., Liang, K. & Yang, Y. The ubiquitin-like modification by ThiS and ThiF in Escherichia coli. Int. J. Biol. Macromol. 141, 351-357 (2019).
20. Burroughs, A. M., Iyer, L. M. & Aravind, L. The natural history of ubiquitin and ubiquitin-related domains. Front. Biosci. Landmark Ed. 17, 1433-1460 (2012).
21. Lehmann, C., Begley, T. P. & Ealick, S. E. Structure of the Escherichia coli ThiS-ThiF complex, a key component of the sulfur transfer system in thiamin biosynthesis. Biochemistry 45, 11-19 (2006).
22. Kranzusch, P. J. et al. Structure-guided reprogramming of human cGAS dinucleotide linkage specificity. Cell 158, 1011-1021 (2014).
23. Kaiser, S. E. et al. Noncanonical E2 recruitment by the autophagy E1 revealed by Atg7-Atg3 and Atg7-Atg10 structures. Nat. Struct. Mol. Biol. 19, 1242-1249 (2012).
24. Yamaguchi, M. et al. Noncanonical recognition and UBL loading of distinct E2s by autophagy-essential Atg7. Nat. Struct. Mol. Biol. 19, 1250-1256 (2012).
25. Schäfer, A., Kuhn, M. & Schindelin, H. Structure of the ubiquitin-activating enzyme loaded with two ubiquitin molecules. Acta Crystallogr. D Biol. Crystallogr. 70, 1311-1320 (2014).
26. Olsen, S. K., Capili, A. D., Lu, X., Tan, D. S. & Lima, C. D. Active site remodelling accompanies thioester bond formation in the SUMO E1. Nature 463, 906-912 (2010).
27. Bernheim, A. et al. Prokaryotic viperins produce diverse antiviral molecules. Nature 589, 120-124 (2021).
28. Doron, S. et al. Systematic discovery of antiphage defense systems in the microbial pangenome. Science 359, eaar4120 (2018).
29. Johnson, A. G. et al. Bacterial gasdermins reveal an ancient mechanism of cell death. Science 375, 221-225 (2022).
30. Gao, J. et al. Identification and characterization of phosphodiesterases that specifically degrade 3′3′-cyclic GMP-AMP. Cell Res. 25, 539-550 (2015).
31. Burroughs, A. M. & Aravind, L. Identification of Uncharacterized Components of Prokaryotic Immune Systems and Their Diverse Eukaryotic Reformulations. J. Bacteriol. (2020) doi:10.1128/JB.00365-20.
32. Oudshoorn, D., Versteeg, G. A. & Kikkert, M. Regulation of the innate immune system by ubiquitin and ubiquitin-like modifiers. Cytokine Growth Factor Rev. 23, 273-282 (2012).
33. Zinngrebe, J., Montinaro, A., Peltzer, N. & Walczak, H. Ubiquitin in the immune system. EMBO Rep. 15, 28-45 (2014).
34. Hu, H. & Sun, S.-C. Ubiquitin signaling in immune responses. Cell Res. 26, 457-483 (2016).
35. Qiu, J. et al. Ubiquitination independent of E1 and E2 enzymes by bacterial effectors. Nature 533, 120-124 (2016).
36. Grau-Bové, X., Sebé-Pedrós, A. & Ruiz-Trillo, I. The eukaryotic ancestor had a complex ubiquitin signaling system of archaeal origin. Mol. Biol. Evol. 32, 726-739 (2015).
37. Hennell James, R. et al. Functional reconstruction of a eukaryotic-like E1/E2/(RING) E3 ubiquitylation cascade from an uncultured archaeon. Nat. Commun. 8, 1120 (2017).
38. Iyer, L. M., Burroughs, A. M. & Aravind, L. The prokaryotic antecedents of the ubiquitin-signaling system and the early evolution of ubiquitin-like beta-grasp domains. Genome Biol. 7, R60 (2006).
39. Eisenacher, K. & Krug, A. Regulation of RLR-mediated innate immune signaling—It is all about keeping the balance. Eur. J. Cell Biol. 91, 36-47 (2012).
40. Crozat, K., Vivier, E. & Dalod, M. Crosstalk between components of the innate immune system: promoting anti-microbial defenses and avoiding immunopathologies. Immunol. Rev. 227, 129-149 (2009).
41. Edgar, R. C. MUSCLE: multiple sequence alignment with high accuracy and high throughput. Nucleic Acids Res. 32, 1792-1797 (2004).
42. Raran-Kurussi, S., Cherry, S., Zhang, D. & Waugh, D. S. Removal of Affinity Tags with TEV Protease. Methods Mol. Biol. Clifton NJ 1586, 221-230 (2017).
43. Punjani, A., Rubinstein, J. L., Fleet, D. J. & Brubaker, M. A. cryoSPARC: algorithms for rapid unsupervised cryo-EM structure determination. Nat. Methods 14, 290-296 (2017).
44. Zheng, S. Q. et al. MotionCor2: anisotropic correction of beam-induced motion for improved cryo-electron microscopy. Nat. Methods 14, 331-332 (2017).
45. Tan, Y. Z. et al. Addressing preferred specimen orientation in single-particle cryo-EM through tilting. Nat. Methods 14, 793-796 (2017).
46. Jumper, J. et al. Highly accurate protein structure prediction with AlphaFold. Nature 596, 583-589 (2021).
47. Govande, A. A., Duncan-Lowey, B., Eaglesham, J. B., Whiteley, A. T. & Kranzusch, P. J. Molecular basis of CD-NTase nucleotide selection in CBASS anti-phage defense. Cell Rep. 35, 109206 (2021).
48. Pettersen, E. F. et al. UCSF Chimera—a visualization system for exploratory research and analysis. J. Comput. Chem. 25, 1605-1612 (2004).
49. Emsley, P., Lohkamp, B., Scott, W. G. & Cowtan, K. Features and development of Coot. Acta Crystallogr. D Biol. Crystallogr. 66, 486-501 (2010).
50. Afonine, P. V. et al. Real-space refinement in PHENIX for cryo-EM and crystallography. Acta Crystallogr. Sect. Struct. Biol. 74, 531-544 (2018).
51. Williams, C. J. et al. MolProbity: More and better reference data for improved all-atom structure validation. Protein Sci. Publ. Protein Soc. 27, 293-315 (2018).
52. Barad, B. A. et al. EMRinger: side chain-directed model and map validation for 3D cryo-electron microscopy. Nat. Methods 12, 943-946 (2015).
53. Kabsch, W. XDS. Acta Crystallogr. D Biol. Crystallogr. 66, 125-132 (2010).
54. Evans, P. R. & Murshudov, G. N. How good are my data and what is the resolution? Acta Crystallogr. D Biol. Crystallogr. 69, 1204-1214 (2013).
55. Evans, P. Scaling and assessment of data quality. Acta Crystallogr. D Biol. Crystallogr. 62, 72-82 (2006).
56. McCoy, A. J. et al. Phaser crystallographic software. J. Appl. Crystallogr. 40, 658-674 (2007).
57. Afonine, P. V. et al. Towards automated crystallographic structure refinement with phenix.refine. Acta Crystallogr. D Biol. Crystallogr. 68, 352-367 (2012).
58. Schindelin, J. et al. Fiji: an open-source platform for biological-image analysis. Nat. Methods 9, 676-682 (2012).
59. Zhou, W., Ryan, J. J. & Zhou, H. Global analyses of sumoylated proteins in Saccharomyces cerevisiae. Induction of protein sumoylation by cellular stresses. J. Biol. Chem. 279, 32262-32268 (2004).
60. Ma, K., Vitek, O. & Nesvizhskii, A. I. A statistical model-building perspective to identification of MS/MS spectra with PeptideProphet. BMC Bioinformatics 13 Suppl 16, S1 (2012).
61. Streich, F. C. & Lima, C. D. Structural and functional insights to ubiquitin-like protein conjugation. Annu. Rev. Biophys. 43, 357-379 (2014).
62. Zheng, N. & Shabek, N. Ubiquitin Ligases: Structure, Function, and Regulation. Annu. Rev. Biochem. 86, 129-157 (2017).
63. Geng, J. & Klionsky, D. J. The Atg8 and Atg12 ubiquitin-like conjugation systems in macroautophagy. ‘Protein modifications: beyond the usual suspects’ review series. EMBO Rep. 9, 859-864 (2008).
64. Taherbhoy, A. M. et al. Atg8 transfer from Atg7 to Atg3: a distinctive E1-E2 architecture and mechanism in the autophagy pathway. Mol. Cell 44, 451-461 (2011).
65. Noda, N. N. et al. Structural basis of Atg8 activation by a homodimeric E1, Atg7. Mol. Cell 44, 462-475 (2011).
66. Hong, S. B. et al. Insights into noncanonical E1 enzyme activation from the structure of autophagic E1 Atg7 with Atg8. Nat. Struct. Mol. Biol. 18, 1323-1330 (2011).
67. Begley, T. P., Xi, J., Kinsland, C., Taylor, S. & McLafferty, F. The enzymology of sulfur activation during thiamin and biotin biosynthesis. Curr. Opin. Chem. Biol. 3, 623-629 (1999).
68. Michelle, C., Vourc'h, P., Mignon, L. & Andres, C. R. What was the set of ubiquitin and ubiquitin-like conjugating enzymes in the eukaryote common ancestor? J. Mol. Evol. 68, 616-628 (2009).
69. Yamada, R. et al. Cell-autonomous involvement of Mab2111 is essential for lens placode development. Dev. Camb. Engl. 130, 1759-1770 (2003).
70. Juang, Y.-C. et al. OTUB1 co-opts Lys48-linked ubiquitin recognition to suppress E2 enzyme function. Mol. Cell 45, 384-397 (2012).
71. Hong, S. B., Kim, B.-W., Kim, J. H. & Song, H. K. Structure of the autophagic E2 enzyme Atg10. Acta Crystallogr. D Biol. Crystallogr. 68, 1409-1417 (2012).
72. Shrestha, R. K. et al. Insights into the mechanism of deubiquitination by JAMM deubiquitinases from cocrystal structures of the enzyme with the substrate and product. Biochemistry 53, 3199-3217 (2014).

### Tables

TABLE 1

| | | CBASS | β5-β6 loop | Disordered C- |
|---|---|---|---|---|
| Protein | PDB ID | Type | length | terminal residues |
| Rhodothermus marinus CdnE | 6E0K | I | 13 | 0 |
| Elizabethkingia meningoseptica CdnE | 6E0M | I | 6 | 1 |
| Flavobacteriacaea CdnE | 6WT8 | I | 13 | 4 |
| Capnocytophaga granulosa CdnE | 6WT9 | I | 17 | 0 |
| V. cholerae DncV | 4TXZ | II | 46 | 23 |
| Enterobacter CdnD | 7LJL | II | 66 | 27 |
| Salmonella CdnD | 7LJM | II | 66 | 27 |
| Bacteroides fragilis CdnB | 7LJO | II (short) | 11 | 50 |
| Bradyrhizobium diazoefficiens CdnG | 7LJN | II (short) | 7 | 49 |
| E. coli CdnC | 6P80 | III | 8 | 0 |
| P. aeruginosa CdnD | 6P82 | III | 5 | 0 |

TABLE 2

| Organism | Gene ID | Accession Number | SEQ ID NO. |
|---|---|---|---|
| Vibrio cholerae C6706 | capV | WP_001133548.1 | 9 |
| Vibrio cholerae C6706 | dncV (cd-ntase) | WP_001901330.1 | 6 |
| Vibrio cholerae C6706 | cap2 | WP_001884104.1 | 2 |
| Vibrio cholerae C6706 | cap3 | WP_001286069.1 | 3 |
| Enterobacter cloacae UCI 50 (a.k.a. | cap4 | WP_032676399.1 | 10 |
| Enterobacter hormaechei subsp. hoffmannii UCI | | | |
| 50) | | | |
| Enterobacter cloacae UCI 50 (a.k.a. | ec cdnD02 (a.k.a. | WP_032676400.1 | 5 |
| Enterobacter hormaechei subsp. hoffmannii UCI | cd-ntase038, cd- | | |
| 50) | ntase) | | |
| Enterobacter cloacae UCI 50 (a.k.a. | cap2 | WP_050010101.1 | 1 |
| Enterobacter hormaechei subsp. hoffmannii UCI | | | |
| 50) | | | |
| Enterobacter cloacae UCI 50 (a.k.a. | cap3 | WP_157903655.1 | 4 |
| Enterobacter hormaechei subsp. hoffmannii UCI | | | |
| 50) | | | |
| Citrobacter freundii UCI 32 | cap5 | WP_000992191.1 | 11 |
| Citrobacter freundii UCI 32 | cd-ntase042 (cd- | WP_032942206 | 12 |
| | ntase) | | |
| Citrobacter freundii UCI 32 | cap2 | WP_014640894.1 | 13 |
| Citrobacter freundii UCI 32 | cap3 | WP_001002160.1 | 14 |
| | | | 15 |
| Escherichia coli UPEC 36 | cd-ntase127 (cd- | WP_052435251.1 | 16 |
| | ntase) | | |
| Escherichia coli UPEC 36 | cap2 | WP_042079576.1 | 17 |
| Escherichia coli UPEC 36 | cap3 | WP_023302104.1 | 18 |
| Escherichia coli UPEC 36 | cap14 | WP_052435252.1 | 19 |

| | | Relative |
|---|---|---|
| | Peak | Peak |
| Peptide | Area | Area |
| Untreated E. cloacae CD-NTase-GFP | | |
| (peptides ending in VSK.G) | | |
| K.ISSTMVSGGIGSGSNGSSGSVSK.G | 8.45E+07 | |
| K.ISSTMVSGGIGSGSN(+.98)GSSGSVSK.G | 3.27E+09 | |
| K.ISSTM(+15.99)VSGGIGSGSNGSSGSVSK.G | 7.86E+06 | |
| Cap3-treated E. cloacae CD-NTase-GFP | | |
| (peptides ending in VSK.G) | | |
| R.FAGIGSGSN(+.98)GSSGSVSK.G | 2.58E+08 | 0.495 |
| A.GIGSGSN(+.98)GSSGSVSK.G | 5.21E+08 | 1.000 |
| G.IGSGSN(+.98)GSSGSVSK.G | 8.89E+05 | 0.002 |
| I.GSGSN(+.98)GSSGSVSK.G | 7.16E+05 | 0.001 |
| I.GSGSNGSSGSVSK.G | 1.48E+05 | 0.000 |
| Untreated V. cholerae CD-NTase-GFP | | |
| (peptides ending in VSK.G) | | |
| K.ISSTMVSGGIGSGSNGSSGSVSK.G | 8.45E+07 | |
| K.ISSTMVSGGIGSGSN(+.98)GSSGSVSK.G | 3.27E+09 | |
| K.ISSTM(+15.99)VSGGIGSGSNGSSGSVSK.G | 7.86E+06 | |
| Cap3-treated V. cholerae CD-NTase-GFP | | |
| (peptides ending in VSK.G) | | |
| K.ISSTM(+15.99)VSGGIGSGSN(+.98)GSSGSVSK.G | 1.03E+08 | 0.054 |
| S.STM(+15.99)VSGGIGSGSN(+.98)GSSGSVSK.G | 6.25E+07 | 0.033 |
| S.TM(+15.99)VSGGIGSGSN(+.98)GSSGSVSK.G | 4.78E+06 | 0.003 |
| T.M(+15.99)VSGGIGSGSN(+.98)GSSGSVSK.G | 5.85E+08 | 0.306 |
| M.VSGGIGSGSN(+.98)GSSGSVSK.G | 5.37E+07 | 0.028 |
| S.GGIGSGSN(+.98)GSSGSVSK.G | 1.88E+07 | 0.010 |
| G.GIGSGSN(+.98)GSSGSVSK.G | 1.91E+09 | 1.000 |
| G.IGSGSN(+.98)GSSGSVSK.G | 9.15E+05 | 0.000 |
| I.GSGSN(+.98)GSSGSVSK.G | 2.95E+06 | 0.002 |

EXTENDED DATA TABLE 1
Summary of Crystallographic data collection and refinement statistics

| | Cap2-CD-NTase 2: 2 complex | Cap2-CD-NTase 2: 1 complex |
|---|---|---|
| | (EMDB-26028) | (EMDB-26066) |
| | (PDB 7TO3) | (PDB 7TQD) |
| Data collection and processing | | |
| Magnification | 165,000 | 165,000 |
| Voltage (kV) | 300 | 300 |
| Electron exposure ( [symbol] /Å [symbol] ) | 65 | 65 |
| Defocus range (μm) | −0.5 to −2.5 | −0.5 to −2.5 |
| Pixel size (Å) | 0.84 | 0.84 |
| Symmetry imposed | C2 | C1 |
| Initial particle images (no.) | 2,554,650 | 2,554,650 |
| Final particle images (no.) | 138,202 | 147,709 |
| Map resolution (Å) | 2.74 | 2.9 |
| FSC threshold | 0.143 | 0.143 |
| Map resolution range (Å) | 2.58-5.47 | 2.71-5.7 |
| Refinement | | |
| Initial model used (PDB code) | de novo | 7TO3 |
| Model resolution (Å) | 2.7 | 2.9 |
| FSC threshold | 0.143 | 0.143 |
| Map sharpening B factor (Å²) | 80 | 77.6 |
| Model composition | | |
| Non-hydrogen atoms | 14764 | 8690 |
| Protein residues | 1850 | 1096 |
| Ligands | 4 Mg [symbol] , 2 AMP, 2 ADP, 2 ATP | 2 Mg [symbol] , 1 AMP, 1 ADP, 1 ATP |
| B factors (Å [symbol] ) | | |
| Protein | 99.97 | 116.24 |
| Ligand | 90.14 | 110.7 |
| R.m.s. deviations | | |
| Bond lengths (Å) | 0.002 | 0.003 |
| Bond angles (°) | 0.544 | 0.519 |
| Validation | | |
| MolProbity score | 1.11 | 1.14 |
| Clashscore | 3.2 | 3.15 |
| Poor rotamers (%) | 0 | 0 |
| Ramachandran plot | | |
| Favored (%) | 98.14 | 97.87 |
| Allowed (%) | 1.86 | 2.13 |
| Disallowed (%) | 0 | 0 |

[symbol]

indicates data missing or illegible when filed

EXTENDED DATA TABLE 2
Summary of Crystallographic data collection
and refinement statistics

| | Cp2 E1-CD-NTase Apo | | Cap2 E1-CD-NTase: AMP | |
|---|---|---|---|---|
| | (PDB 7TSX) | | (PDB 7TSQ) | |
| Data collection | | | | |
| Space group | P2₁2₁2₁ | | P2₁2₁2₁ | |
| Cell dimensions | | | | |
| a, b, c (Å) | 50.26, 76.55, 127.63 | | 49.69, 76.35, 126.90 | |
| a, b, g (°) | 90, 90, 90 | | 90, 90, 90 | |
| Resolution (Å) | 128-1.77 | (1.80-1.77)* | 127-2.11 | (2.18-2.11) |
| R_{merge} | 0.087 | (1.315) | 0.146 | (0.860) |
| I/sI | 14.1 | (1.3) | 15.3 | (2.5) |
| Completeness (%) | 99.9 | (98.7) | 99.6 | (97.0) |
| Redundancy | 6.7 | (6.5) | 6.5 | (5.8) |

| | | |
|---|---|---|
| Refinement | | |
| Resolution (Å) | 65.65-1.77 | 65.42-2.11 |
| No. reflections | 48,900 | 28,241 |
| R_{work}/R_{free} | 16.95/19.15 | 20.22/22.93 |
| No. atoms | 6970 | 6830 |
| Protein | 6585 | 6576 |
| Ligand/ion | 0 | 36 |
| Water | 385 | 218 |
| B-factors | 35.68 | 47.26 |
| Protein | 34.8 | 46.77 |
| Ligand/ion | | 73.59 |
| Water | 43.22 | 48.96 |
| R.m.s. deviations | | |
| Bond lengths (Å) | 0.004 | 0.006 |
| Bond angles (°) | 0.64 | 0.78 |

*A single crystal was used for each dataset.

1. A system of generating a fusion peptide comprising:
a first peptide;
a Cap2 enzyme having a target recognition motif,
a target peptide coupled with said target recognition motif;
wherein said Cap2 enzyme ligates said first peptide to said target peptide, forming a fusion peptide.

2. The system of claim 1, further comprising an intermediary peptide coupling said first peptide with said Cap2 enzyme.

3. The system of claim 1 wherein said first peptide comprises an intermediary peptide recognition motif.

4. The system of claim 2, wherein said intermediary peptide comprises a CD-NTase peptide, or a fragment or variant thereof.

5. The system of claim 4, wherein said CD-NTase peptide is selected from SEQ ID NO.'s 5-6, 12 or 15, or a fragment or variant thereof.

6. The system of claim 3, wherein said intermediary peptide recognition motif comprises a CD-NTase recognition motif.

7. (canceled)

8. The system of claim 1, wherein said Cap2 enzyme is selected from SEQ ID NO.'s 1-2, 13 or 16, or a fragment or variant thereof.

9. The system of claim 1, wherein said target recognition motif comprises an antibody, or a fragment thereof, or an engineered protein binding motif.

10-11. (canceled)

12. The system of claim 1, wherein the amino acid sequence of said first peptide and said target peptide is preserved in said fusion peptide.

13-17. (canceled)

18. The system of claim 1, wherein said Cap2 enzyme comprises a homodimer.

19-51. (canceled)

52. An isolated composition comprising:
a fusion peptide including:
a first peptide;
a Cap2 enzyme, or a fragment or variant thereof, having a target recognition motif,
a target peptide; and
an intermediary peptide.

53. The composition of claim 52, wherein said first peptide comprises an intermediary peptide recognition motif.

54. The composition of claim 52, wherein said intermediary peptide comprises a CD-NTase peptide, or a fragment or variant thereof.

55. The composition of claim 54, wherein said CD-NTase peptide is selected from SEQ ID NO.'s 5-6, 12 or 15, or a fragment or variant thereof.

56. The composition of claim 53, wherein said intermediary peptide recognition motif comprises a CD-NTase recognition motif.

57. (canceled)

58. The composition of claim 52, wherein said Cap2 enzyme is selected from SEQ ID NO.'s 1-2, 13 or 16, or a fragment or variant thereof.

59. The composition of claim 52, wherein said target recognition motif comprises an antibody, or a fragment thereof, or an engineered protein binding motif.

60. (canceled)

61. The composition of claim 52, wherein the amino acid sequence of said first and target peptides are preserved in said fusion peptide.

62. The composition of claim 52, wherein said Cap2 enzyme comprises a homodimer.

63-101. (canceled)


SEQUENCE LISTING (20 sequences)

SEQ ID NO: 1 - protein, 600 aa, Enterobacter cloacae
MSTVVQQVPAELQAALTLINNDPRMRTNNAWALSADKRWSLKFTAELSVPCSSFMPDNSVWHLVLWQEETLIRIEVYPDKSEGISATFQHQNYNFSDASTREWTSGNPCLENTPTVFGRNLWGLEPEALLDRISWRLSRLLLWIDAAAQEKLATTGDAVELPAFPDQSPFTVIGFSEQIDDLPFWASKTGEWGFASSTGLPGAHGTLFLREFLDNKGKLIRTTKWSPFMRKGARTTNAVWSVLPTLPVLAPWQAPRTWQELSHCFAQCGLSLPDLFSDIGRSVRALRKQRAPGLLLLGFPLENKIGDEPARIHWLALRLAGLSNTMTKRPGFRPTERNRRTWDREQPLSQEPIRWVRTQNWAADQLRTRGEAANDIRSKKVLIIGAGSLGSMIAENLMRIGVVSQGILDADLLQTGNLSRHALTMTSVGHNKAAALVEHLNRILPDASARSFSCAFPPESEVAKNSLRQYDVIIDCTGDDGVLKSLAAFDWKSEKIFISLAMTWRAEGLFAFAASETSFPVTDASSRFNASASPEIDMDEARIEGIGCWHPVFPARADDVQLWAAVGTKFICRVVSAPGRIYEYFKQMPDGTVEKEPHEY

SEQ ID NO: 2 - protein, 584 aa, Vibrio cholerae
MKQELHHTLLGCGFRYTPAKQMPKGILLDTKSRRKGYYVKEYSTKGGVFVIALVLWNDPHIQLPFAYILQQPEQYKGRLLPHINFGFCLCYVTQMEADWNSNDLKSTYQDVDEQIQLTLDNSVASVESGTSNDVELEGEFSAYWQSEEELYLLAKPSRKAQLKAHLVEAELSSGSIRREYVAACSEQSEELVKWLNQRKFDESSLQEVSITTHCISVKPNRLAGVNWPPSCLREVLSWLKLVDYSAHARTVTLLMAKRTKRHILLFDVEGQDELAVYLELNLDVIGKRYFGRNAARKRNINNEAALLGGKFVSANFKRLGVTRADRDTLLSRNQSRPDVGNLSQKRIALIGCGTIGGYLAELLLRSGAGCGENYFHLYDNDSFKPHNFARHSLTAHNFGLAKSIALANSLKEAVHIAQSIKGIDRQFPIQADVLSKYDIVIDATGRPPVSKRLAAVARTLIADIRPVLIHAFNDGNGRASKVLVDDGRCCYGCMMADPAVYRNNIDLRFEGIDLAKEKHISCGSTYTPYDAAVSHITAALAQEAVLNTLEHTLPWNYSEHMLDGSRSKKPRTLKRFSGCNICDE

SEQ ID NO: 3 - protein, 156 aa, Vibrio cholerae
MMSDVELVFKDESDCLVVIMGHVVTRLLSYRQLHHLTPESAGVLIGERRGQHLVVCDISEPGSGDIRQRCRVDRRGVHHQSRVNEAFERSAGTHLYLGEWHTHPEDRPFPSATDRHSWRRNIVSDESMLLLIVGRKDFWLGKKERELITVFKKIES

SEQ ID NO: 4 - protein, 180 aa, Enterobacter cloacae
MNILSRCLMALSRKSRMSIKDVTFSSEGALYTIVISSSTIEQMVSECLKAGVNETGGILIGSYSEDSSTAMIVEATTRPADSLAGRTTFQRGVRGLRPLLHARWKTGLYYVGEWHFHPGGSPEPSGDDFRSMTSIAANSDYQCLEPVMIILGGDPAGSYSLSASVSPRGDAPIRLREVLI

SEQ ID NO: 5 - protein, 381 aa, Enterobacter cloacae
MELQPQFNEFLANIRPTDTQKEDWKSGARTLRERLKNFEPLKEIVVSTFLQGSIRRSTAIRPLGDKRPDVDIVVVTNLDHTRMSPTDAMDLFIPFLEKYYPGKWETQGRSFGITLSYVELDLVITAIPESGAEKSHLEQLYKSESVLTVNSLEEQTDWRLNKSWTPNTGWLSESNSAQVEDAPASEWKAHPLVLPDREKNEWGRTHPLAQIRWTAEKNRLCNGHYINLVRAVKWWRQQNSEDLPKYPKGYPLEHLIGNALDNGTTSMAQGLVQLMDTFLSRWAAIYNQKSKPWLSDHGVAEHDVMARLTAEDFCSFYEGIASAAEIARNALASEEPQESAQLWRQLFGSKFPLPGPQGGDRNGGFTTPSKPAEPQKTGRFA

SEQ ID NO: 6 - protein, 436 aa, Vibrio cholerae
MRMTWNFHQYYTNRNDGLMGKLVLTDEEKNNLKALRKIIRLRTRDVFEEAKGIAKAVKKSALTFEIIQEKVSTTQIKHLSDSEQREVAKLIYEMDDDARDEFLGLTPRFWTQGSFQYDTLNRPFQPGQEMDIDDGTYMPMPIFESEPKIGHSLLILLVDASLKSLVAENHGWKFEAKQTCGRIKIEAEKTHIDVPMYAIPKDEFQKKQIALEANRSFVKGAIFESYVADSITDDSETYELDSENVNLALREGDRKWINSDPKIVEDWFNDSCIRIGKHLRKVCRFMKAWRDAQWDVGGPSSISLMAATVNILDSVAHDASDLGETMKIIAKHLPSEFARGVESPDSTDEKPLFPPSYKHGPREMDIMSKLERLPEILSSAESADSKSEALKKINMAFGNRVTNSELIVLAKALPAFAQEPSSASKPEKISSTMVSG

SEQ ID NO: 7 - protein, 8 aa, Vibrio cholerae
ISSTMVSG

SEQ ID NO: 8 - protein, 8 aa, Enterobacter cloacae
PQKTGRFA

SEQ ID NO: 9 - protein, 355 aa, Vibrio cholerae
MPNPPEYEHLKNQVRILSLNGGGARGLFTISLLAEIERIIEEKQGINGFKVGDYFDLITGTSIGGILALGLAYGKSARELEDVFRKQAGYIFPEQKYPRFFPVFRRRYRLARGPLYDSKPLAKTIASMVGEESTFNDLKCRVLIPTVNLSTGKPQFFKTPHNPEFHRDGRIKLIDAALATSAAPTYFAPHYCVDLDSYFADGGLVANNPSFIGLHEVFRDMATDFPEAKVSDVKILNVGTLGEEYSLSPSSLAGKSGYLGLWGMGERLVLSAMAANQELHKAMLLREFATHDAIGNFVRLDNNIPHEAASDITLDNASASSLSNLASRGRQLATEEFTKNKALADFFKVPARKFK

SEQ ID NO: 10 - protein, 499 aa, Enterobacter cloacae
MATSVLANWHGHDYQARYFWIEASRLKNPQQDFVVEVSYEADGPKAFDDVITRYNPPRRSTGPDRIQADYYQIKFHVTQAASFGFEDLIDPAFIGAETFSILERLKQAKGTEPANSAFHLVTTDRIIDEDPLGEIISNVDGSIRLDKLFDGTTDRSRKGKVRKLWRQHLKLSTDQELEQVLSGFHIQQSQPTLEAMREKVNTCFQIIGLITCETSSDFRFDGAARALRSQERYRFTREQFTALCEEENWIRSEAPESFRNVALRSFSDGPLDIMDALPEHTLSLLSLFEGRFPSPGIEWNDVIKPQVETFLTGIRQTERKVRLYLNTHSSIAMLAGKCLGHKSGVEIELVQKGRMGDSIWSENESQDEPDAVIETETVGTGSDVAVVLSITRNALPKARAYILENQPDIGRIIHVTPANGHGQRSVKNGSHAVAIAEQVSDVVMDADLPVEASLHIFSAAPNAVNFYLGQHTDFLGTCVFYEFDFQRQRDGSYLPSFKV

SEQ ID NO: 11 - protein, 391 aa, Citrobacter freundii
MNAKKKADDSSGRFNTSPEVRTLVWIRAAGHCELCGTDLTHDFRIGTTMKWGEVAHILPASPKGPRGNATHSVEEALARTNDSENLMLLCPGCHDRVDRDGDNYPEDDLSGLHSACLTRIRLAASTPGEERAIPVIVQSQHHQTLVAIPAQALLTAMSAEGLTAQCHPVTVVFPEPSSRGRDAGYWQAIKDLITEKLEAGLARRGGQFGDKPALAMVGLADIPALFFLGQTIGDRYKRFQYSFNREHILRWPDTDALPPAFISSELPQGEGPVALVLSLSAAVPRGDVTAALPGARIVEFTVPEPSYAMVTNRRVIHAFRDELQKLLSQLEATTTQAIHVFAAVPAALAIEFGALLTTQHQHPYIIFDRDKNNANQFRPMLSIGCTEEQRP

SEQ ID NO: 12 - protein, 415 aa, Citrobacter freundii
MSNEQTKHRSWEYFLLRAARKISLSAAQYSVIDARYSQLEKILSAADDPLLADAHIFPQGSMRLQTTINPVPGAPADLGTIDADAIVWLPHARGIDARTVLEVIERRFQEGSRVQEDIQQLRRGVRIVYADENPGFHIDVTPARPCHHNEQSDGLGMLEVPDREHGWKASSPIPYADWLHDASKQDIMLEHVVEFNKSRAAMDSATQAPLPEYKEYQKDDPLRASIKLMKRHRDEWAIRTKNEGYRPISAVITTLATHAYLDVVAQSEYTAFTPLQAILAIVNRMPDHIHRYSNEYYVCNPEDNGENFAEKWNRPDEGYKYVDAFNKWHASARSALTLGLDSHASTETFAKAVQEQFGIGPTFVREVNESIPANWTMPGRQDGVTRNSVSMGSLFGSSVSSNQSQANVAPVGRLG

SEQ ID NO: 13 - protein, 587 aa, Citrobacter freundii
MKDEMIQRGISALKDYLGADNASALLRPAELRVGEAVAFYFPLPVDYTGLERRLRIGFPNDFPSESPSLQVEPSPWLVWPHAMTSGLCLHGFREKPVTGSPEKIVQDSLSRFASIVSFSLENADPARRETEFQNEISTYWLWQLKRSARNLILLGEPESGSVLYVVSDPRYTGHTGLNPVWVSSDKNAIRQHFRRATGRSVVIRSPHEAGFFVKLTSFPGIKVPEPHAFLEWLAPHISEESLSAMSKWSEKSSTLLSRWVVMALPGGDGAARFTVNLCTRKKETDRTHFYGLRSSRRQSGLKKEGPPASILSSRVNVIDRGAFFSRDRSNTAKTLENSHVVFVGVGSLGSAVSIQLARAGLGRLTLIDPDRLESPNLGRHMLGAEDLGKFKSQAMRHRLLQDLPVLDVTALDTYIEWVMGQKPDIFEDVDLVIITTADWESESALWEKKASGAKWGLIQAWSEPHTLVGHALIAPEGRFDARYLFSDRGDFRHRFTDWPDGGVVPLPACGESFIPGGSAGMNGVATMVTQAAIRYLTTVGDVTQWHSSVYRPDEAGVLGGRYTGPVLPEGVVHSVFERNWPKPGQNA

SEQ ID NO: 14 - protein, 165 aa, Citrobacter freundii
MNEIAWCWAWPDMNAELQVGRQVSEMFGHYRQSQGENERGGQLFVDPANPGGIVLCHASAPHPADRAGRTWLELNNDRCRTEIENANRKGLRLVGYWHTHPQAVPHISGTDIASFARFARQNARDLPHPVAVIVGTSPSTTGIKAWIFREGRYQEAVYTASLSRP

SEQ ID NO: 15 - protein, 367 aa, Escherichia coli
MFSQVIECYDFYFVVKNYQAIYYLYLLTKKDILIMATAQTQFENFHSKILMGYDDNEPLRDRRDTLIQELKDKLSSDVPNMKFFHQGSYALHTGINPLNGNPDIDIGIIFECDPNDEDYQDPLKLKKIVRDALNTKNRTVKIKRPCVTVEYLRDGEPIYHIDLALYSCEYGDSDGQTFLARGKETSSAEEIEWQISSARELTDVILNKYTDSSHKKQFRRIVRYLKRWKDEKLGHKNTPSIGLTVAAYELFSANFDFVSGKAQDLLALLSLVNSMLNNWGGDDRLHIYLPVEPSTDLFERMSDNQMKDLKTKLEKLSAVLIEARDQPDTHEACKLLKAQFGDDFPVPDKSETTKSSSSSVVPAGRSA

SEQ ID NO: 16 - protein, 544 aa, Escherichia coli
MVCKCSFEEAVSFSLESYGCKHIKVAKQSGFVTSWFFSLDFKNYLWDFELISTTNDLSTLPSLRWIRPHKVWGWPHINSAGIICFEDGQGIEFEPKDIAGVITWVLDEGTNLLKKYWNMGKAKRKREFFDELDGYLKSMGVPEVLQIDNYGNKDVDIAYAEVEYPKIGKERGVVRRVFRSSLSYVPSRNSCEKLVVMTISYKKIPPITGQVNESWWTKIYDQLSQEQLLLLEGKRCRGIIFRVKNSYGYCEFLIHWGQGKYNNKRWLGRLYRIQFASWEYVTQRTGIPASRQRIAVVGVGAVGSRIAEMALLSGASDIYLVDDDKFSADNMGRHLLGLNSVGKYKVDELSIHFKSRIPGVNIHPFRVRAQKYFSQDNNDYDIIFLATGNSSLEKGIILRAFKEKWNSVLVSVSVEAYGVGGQIIIMHTNIKGCLYCLYIDNETNTLMPNLQGGFIEPGQKISKQLTGCGAFTPFSALNASKTAMLAYEYAVSKKYGYLRWAGNNDIAMSEGIILTPAYFNIFNENTPSFLHSEQIAKKGCPCCG

SEQ ID NO: 17 - protein, 167 aa, Escherichia coli
MLRIIESGQLIWNIEGIATGCLSKKVITHLLNFVQKGRYAHESGGVLLGYIDSDTNGLLVENATFPGSGDCSSRTSFYRSSRHQEDAESWISLTNGKGTHLGLWHTHPELTPTPSDIDMADYNNVIKNATLNSGLIYIIIGTQELKIWYGIKPCQLIPVGTISLDKL

SEQ ID NO: 18 - protein, 369 aa, Escherichia coli
MNKHINHFITSLIDWLFRVKTVPAILLKAGLLCLSFSLIGGWALKVKVPLEDGDFIFDYDSSGFTPALITYITFVLAIGLILTSALIYINVYFRENRLADKKKLISIESRGLRDGAGEPLSKYIPQKNKVRPIEIILDLRQKVEDGFIVKPDVAIRKILTLQEQLESHTSQYSREYIEVYFGGLTPVPLNVLNGIILDDECKITIMDWDRHSSEWKTLDKEDDEERYTIQGVKDVQATDNEVLLVISSSYLIDSSLIQKQFPSMKLVTMFLPAINTDNLWSEIKQVELGKQFLHAVADLSGLGVKRIHLFLAAPNSLCFRFGRLYDKRNLPELVVYQYERNSDVTYPWGILMPVAGIQVPEVIHCKRDI

SEQ ID NO: 19 - protein, 8 aa, Citrobacter freundii
VAPVGRLG

SEQ ID NO: 20 - protein, 8 aa, Escherichia coli
VVPAGRSA